(19) United States
(12) Patent Application Publication
(10) Pub. No.: US 2012/0263690 A1
(43) Pub. Date: Oct. 18, 2012

(54) BIOCONTROL MICROORGANISMS

(76) Inventor: Eudes De Crecy, Gainesville, FL (US)

(21) Appl. No.: 13/399,497

(22) Filed: Feb. 17, 2012

Related U.S. Application Data
(63) Continuation of application No. PCT/US10/45808, filed on Aug. 17, 2010.
(60) Provisional application No. 61/234,613, filed on Aug. 17, 2009.
(60) Provisional application No. 61/300,402, filed on Feb. 1, 2010.
(60) Provisional application No. 61/303,288, filed on Feb. 10, 2010.

(51) Int. Cl. A01N 63/00; C12N 15/01; A01N 63/04

(52) U.S. Cl. 424/93.21; 424/93.2; 435/440

(57) ABSTRACT

Methods, devices, and compositions described herein are directed to artificially evolving an organism for use as a biocontrol agent. Methods, devices, and compositions described herein are useful for evolving a microorganism to acquire traits not naturally associated with the microorganism. The artificial evolution process can utilize culture methods and devices designed to accommodate particular culture methods described herein. The organism can be artificially evolved for a characteristic such as ultraviolet light tolerance, chemical tolerance, thermotolerance, enhanced growth rate on a target carbon source, host specific growth, modified sporulation characteristics or modified spores.

### CROSS-REFERENCE

This application claims the benefit of U.S. Provisional Application No. 61/234,613, filed Aug. 17, 2009, No. 61/300,402, filed Feb. 1, 2010, and No. 61/303,288, filed Feb. 10, 2010, which applications are incorporated herein by reference in their entirety.

### BACKGROUND OF THE INVENTION

Microorganisms are useful hosts for various purposes as they are readily available and are generally considered to be easily amenable compared to animal cells. A variety of modifications has been sought to accommodate agricultural, industrial, or other needs, using conventional genetic modification with mixed success. In part this is due to the genetic complexity of desired traits or phenotypes, which may be affected by multiple genes and transcriptional regulators.

Additionally, the natural habitat of a microorganism does not necessarily coincide with the environmental condition in which the microorganism can be useful. Thus, adapting a microorganism to a habitat that is different than its wild-type habitat is sometimes a required task to turn a microorganism into a useful vehicle.

Adapting a microorganism to artificially acquire a trait, such as thermotolerance, host specificity, UV tolerance or another desired trait can be beneficial. For example, strains with beneficial traits but that do not actively grow at ambient temperatures can be adapted to grow at ambient temperatures in order to use the strain for field applications, such as an open field cultures.

Further, a microorganism can be evolved as a biocontrol agent to provide a natural way to control pests, such as insects. Candidate microorganisms include bacteria, viruses, alga, fungi such as entomopathogenic fungi, or a microorganism capable of sporulation. Some fungi have the ability to penetrate insect's cuticle and are pathogenic to host insects.

Consequently, there is an interest in methods that can artificially evolve a microorganism to have improved performance as a biocontrol agent. Adapting a microorganism to artificially acquire traits, such as thermotolerance, ultra-violet light tolerance, enhanced growth rates, host specificity, chemical resistance or modified sporulation, are disclosed herein.

### SUMMARY OF THE INVENTION

In one aspect, described herein is a method of controlling a pest comprising: applying a microorganism artificially evolved to acquire a trait that is not naturally associated with said microorganism to an area affected by pest infestation, wherein said trait increases said microorganism's ability to inhibit a pest; and inhibiting said pest with said microorganism. In one embodiment, said trait is enhanced tolerance to ultraviolet light. In another embodiment, said trait is enhanced tolerance to chemical. In another embodiment, said trait is a pesticide. In another embodiment, said trait is an herbicide. In another embodiment, said trait is a fungicide In another embodiment, said trait is thermotolerance. In another embodiment, said thermotolerance is enhanced tolerance temperatures higher than said microorganism's normal temperature range. In another embodiment, said trait is enhanced tolerance temperatures lower than said microorganism's normal temperature range. In another embodiment, said trait is enhanced growth rate on a target carbon source. In another embodiment, said trait is enhanced growth rate on a target nitrogen source. In another embodiment, said trait is enhanced host specific growth. In another embodiment, said trait is modified sporulation characteristics. In another embodiment, said trait is modified spores. In another embodiment, said trait is an ability to increase production of an enzyme wherein said enzyme is naturally produced in said strain. In another embodiment, said trait is an ability to constitutively produce an inducible enzyme in said strain. In another embodiment, said trait an ability to induce expression of an enzyme in a condition not known to be inducible for said enzyme in said strain. In another embodiment, said trait is an ability to survive on food sources not naturally utilized in said strain. In another embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae In another embodiment, said bacterium is E. coli. In another embodiment, said E. coli is adapted from the strain MG1655. In another embodiment, the rate of growth of said microorganism at 35.5° C. exceeds that of a naturally occurring strain. In another embodiment, the rate of growth of said microorganism at 37° C. exceeds that of a naturally occurring strain. In another embodiment, the rate of growth of said microorganism in sunlight exceeds that of a naturally occurring strain. In another embodiment, the rate of growth of said microorganism in the presence of a chemical exceeds that of a naturally occurring strain. In another embodiment, said chemical is an herbicide. In another embodiment, said chemical is a pesticide. In another embodiment, said chemical is a fungicide. In another embodiment, the rate of growth of said microorganism on said host exceeds that of a naturally occurring strain. In another embodiment, the host specificity of said microorganism exceeds that of a naturally occurring strain. In another embodiment, the rate of growth of said microorganism from a spore stage exceeds that of a naturally occurring strain. In another embodiment, said pest is an insect. In another embodiment, said pest is grasshoppers, locusts, cockchafers, grubs, borers or malaria-vectoring mosquitoes. In another embodiment, said microorganism was artificially evolved by continuously culturing said microorganism under conditions designed to select for said trait.

In another aspect, described herein herein is an artificially evolved microorganism that is artificially evolved to acquire a trait that is not naturally associated with said microorganism, wherein said trait increases said microorganism's ability to inhibit a pest, wherein said microorganism is artificially evolved by continuously culturing said microorganism under conditions designed to select for said trait. In one embodiment, said trait is enhanced tolerance to ultraviolet light. In another embodiment, said trait is enhanced tolerance to a chemical. In another embodiment, said trait is a pesticide. In another embodiment, said trait is an herbicide. In another embodiment, said trait is a fungicide In another embodiment, said trait is thermotolerance. In another embodiment, said thermotolerance is enhanced tolerance temperatures higher than said microorganism's normal temperature range. In another embodiment, said thermotolerance is enhanced tolerance temperatures lower than said microorganism's normal temperature range. In another embodiment, said trait is enhanced growth rate on a target carbon source. In another embodiment, said trait is enhanced growth rate on a target nitrogen source. In another embodiment, said trait is enhanced host specific growth. In another embodiment, said trait is modified sporulation characteristics. In another embodiment, said trait is modified spores. In another embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, the rate of growth of said microorganism at 35.5° C. exceeds that of a naturally occurring strain. In another embodiment, the rate of growth of said microorganism at 37° C. exceeds that of a naturally occurring strain. In another embodiment, the rate of growth of said microorganism in sunlight exceeds that of a naturally occurring strain. In another embodiment, the rate of growth of said microorganism in the presence of a chemical exceeds that of a naturally occurring strain. In another embodiment, said chemical is an herbicide. In another embodiment, said chemical is a pesticide. In another embodiment, said chemical is a fungicide. In another embodiment, the rate of growth of said microorganism on said host exceeds that of a naturally occurring strain. In another embodiment, the host specificity of said microorganism exceeds that of a naturally occurring strain. In another embodiment, the rate of growth of said microorganism from a spore stage exceeds that of a naturally occurring strain. In another embodiment, said pest is an insect. In another embodiment, said pest is a grasshopper, locust, cockchafers, grub, borer, ant, mite or mosquito.

In another aspect, described herein is a method of artificially evolving a microorganism for enhanced tolerance to ultraviolet light, comprising: administering a microorganism into a flexible tubing wherein said tubing is subdivided by an operation of a gate into one or more discreet chambers; culturing said microorganism; exposing said organism to ultraviolet light; and continuously culturing said microorganism in said chamber until said organism's tolerance to said ultraviolet light has increased. In one embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, said microorganism is capable of sporulation. In another embodiment, said microorganism is exposed to ultraviolet light with a wavelength between 10-400 nm. In another embodiment, said microorganism is exposed to ultraviolet light that is incrementally increased in intensity over time. In another embodiment, said microorganism is exposed to ultraviolet light wavelengths that are incrementally increased in wavelength over time. In another embodiment, said microorganism is continuously exposed to ultraviolet light. In another embodiment, said microorganism is intermittently exposed to ultraviolet light.

In another aspect, described herein is a method of artificially evolving a microorganism for enhanced tolerance to a chemical, comprising: administering a microorganism into a flexible tubing wherein said tubing is subdivided by an operation of a gate into one or more discreet chambers; culturing said microorganism; exposing said microorganism to a chemical; and continuously culturing said microorganism in said chamber until said microorganism's tolerance to said chemical has increased. In one embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, said chemical is an herbicide. In another embodiment, said chemical is a pesticide. In another embodiment, said chemical is a fungicide. In another embodiment, said microorganism is exposed to a incrementally increasing concentrations of said chemical over time. In another embodiment, said microorganism is continuously exposed to said chemical.

In another aspect, described herein is a method of artificially evolving a microorganism for enhanced thermotolerance, comprising: administering a microorganism into a flexible tubing, wherein said tubing is subdivided by an operation of a gate into one or more discreet chambers; culturing said microorganism; exposing said microorganism to a higher or lower temperature than at which it typically grows; and continuously culturing said microorganism in said chamber until said microorganism's tolerance to said temperature has increased or decreased. In one embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, said temperature is about 48° C. In another embodiment, said temperature ranges from 40° C. to 70° C. In another embodiment, said temperature ranges from about 5° C. to about 70° C. In another embodiment, said temperature is incrementally changed over time from 44° C. to 49.7° C. In another embodiment, said temperature is about 37° C. In another embodiment, said temperature is incrementally increased from about 32° C. to about 37° C. In another embodiment, incremental change comprises an increase in temperature of about 1 degree increment over time. In another embodiment, said temperature is incrementally decreased from about 25° C. to about 5° C. In another embodiment, incremental change comprises a decrease in temperature of about 1 degree increment over time.

In another aspect, described herein is a method of artificially evolving a microorganism for an enhanced growth rate on a target carbon source, comprising: administering a microorganism into a flexible tubing wherein said tubing is subdivided by an operation of a gate into one or more discreet chambers; culturing said microorganism; exposing said microorganism to conditions that enhance said microorganism's growth rate on a target carbon source; and continuously culturing said microorganism in said chamber until said microorganism's growth rate on said target carbon source has increased. In another embodiment, said microorganism is a bacterium. In one embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, said microorganism is cultured with said target carbon source. In another embodiment, said target carbon source comprises components of a host insect. In another embodiment, said microorganism is exposed to incrementally increasing amounts of said target carbon source. In another embodiment, said microorganism is continuously exposed to said target carbon source. In another embodiment, said microorganism is exclusively exposed to a target carbon source that consists of components of a host insect.

In another aspect, described herein is a method of artificially evolving a microorganism for an enhanced growth rate on a target nitrogen source, comprising: administering a microorganism into a flexible tubing wherein said tubing is subdivided by an operation of a gate into one or more discreet chambers; culturing said microorganism; exposing said microorganism to conditions that enhance said microorganism's growth rate on a target nitrogen source; and continuously culturing said microorganism in said chamber until said microorganism's growth rate on said target nitrogen source has increased. In one embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, said microorganism is cultured with said target nitrogen source. In another embodiment, said target nitrogen source comprises components of a host insect. In another embodiment, said microorganism is exposed to incrementally increasing amounts of said target nitrogen source. In another embodiment, said microorganism is continuously exposed to said target nitrogen source. In another embodiment, said microorganism is exclusively exposed to a target nitrogen source that consists of components of a host insect.

In another aspect, described herein is a method of artificially evolving a microorganism for host specific growth, comprising: administering a microorganism into a flexible tubing wherein said tubing is subdivided by an operation of a gate into one or more discreet chambers; culturing said microorganism; exposing said microorganism to conditions that enhance said microorganism's host specific growth; and continuously culturing said microorganism in said chamber until said microorganism's specificity to grow on said host has increased. In one embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said bacterium is E. coli. In another embodiment, said E. coli is adapted from the strain MG1655. In another embodiment, said microorganism is cultured on a target carbon source. In another embodiment, said microorganism is cultured on a target nitrogen source. In another embodiment, said microorganism is cultured with components of a host insect. In another embodiment, said microorganism is exposed to incrementally increasing amounts of said components of a host insect over time. In another embodiment, said microorganism is continuously exposed to said components of a host insect. In another embodiment, said microorganism is exclusively exposed to a target carbon source that consists of components of a host insect.

In another aspect, described herein is a method of artificially evolving a sporulating microorganism to modify its sporulation characteristics, comprising: administering a sporulating microorganism into a flexible tubing wherein said tubing is subdivided by an operation of a gate into one or more discreet chambers; culturing said sporulating microorganism; exposing said sporulating microorganism to conditions that modify its sporulation characteristics or spores; and continuously culturing said microorganism in said chamber until said microorganism's sporulation characteristics are modified. In one embodiment, said microorganism is a bacterium. In another embodiment, said microorganism is a virus. In another embodiment, said microorganism is an alga. In another embodiment, said microorganism is a fungus. In another embodiment, said microorganism is an entomopathogenic fungus. In another embodiment, said microorganism is M. anisopliae, M. flavoviridae, or Beauveria bassiana. In another embodiment, said microorganism is M. anisopliae. In another embodiment, said microorganism is induced to form spores. In another embodiment, said microorganism is periodically induced to form spores. The method of claim 180 or 181, wherein said induction comprises drying out said chamber.

In another aspect, described herein is a method of artificially evolving a strain of M. anisopliae to acquire one or more traits not naturally associated with M. anisopliae comprising: placing one or more naturally occurring strains of M. anisopliae in a flexible tubing wherein said tubing is subdivided by an operation of a gate into one or more discreet chambers; placing said strains under a culture condition; allowing said strains to grow continuously in said chamber under said culture condition; sampling said strains; and characterizing said sampled strains for biological properties that are not naturally associated with said strains. In one embodiment, said trait is enhanced tolerance to ultraviolet light. In another embodiment, said trait is enhanced tolerance to chemical. In another embodiment, said trait is a pesticide. In another embodiment, said trait is an herbicide. In another embodiment, said trait is a fungicide. In another embodiment, said trait is thermotolerance. In another embodiment, said thermotolerance is enhanced tolerance temperatures higher than said microorganism's normal temperature range. In another embodiment, said thermotolerance is enhanced tolerance temperatures lower than said microorganism's normal temperature range. In another embodiment, said trait is enhanced growth rate on a target carbon source. In another embodiment, said trait is enhanced growth rate on a target nitrogen source. In another embodiment, said trait is enhanced host specific growth. In another embodiment, said trait is modified sporulation characteristics. In another embodiment, said trait is modified spores. In another embodiment, said trait is an ability to increase production of an enzyme wherein said enzyme is naturally produced in said strain. In another embodiment, said trait is an ability to constitutively produce an inducible enzyme in said strain. In another embodiment, said trait is an ability to induce expression of an enzyme in a condition not known to be inducible for said enzyme in said strain. In another embodiment, said biological property is an ability to survive on food sources not naturally utilized in said strain.

In another aspect, described herein is a method of artificially evolving a strain of M. anisopliae, M. flavoviridae, or Beauveria bassiana to enhanced thermotolerance by continuously culturing said strain under a condition wherein said condition comprising incrementally increasing culture temperature by 1° C., wherein said strain grows robustly at 37 Celsius, and wherein said strain is produced inhibits grasshoppers, locusts, cockchafers, grubs, borers or malaria-vectoring mosquitoes infestation.

In another aspect, described herein is a device for adapting an microorganism for ultraviolet light tolerance, chemical tolerance, thermotolerance, enhanced growth rate on a target carbon source, enhanced growth rate on a target nitrogen source, host specific growth, modified sporulation characteristics or modified spores comprising: a flexible tubing wherein said tubing is subdivided by an operation of a gate into one or more discreet chambers, wherein one or more said gates are located in a fixed distance across longitudinal length of said tubing; one or more flywheels functionally connected to motors wherein said gate is mounted on the surface of said flywheel; a sampling port functionally connected with said flexible tubing wherein a sample of culture can be withdrawn through said sampling port; one or more inlets and outlets wherein said inlets and outlets allow air or culture media to be transported into said flexible tubing; and a timing device wherein said device can instruct the movement of flywheel into user determined direction.

In another aspect, described herein is a device for adapting an organism for ultraviolet light tolerance, chemical tolerance, thermotolerance, enhanced growth rate on a target carbon source, enhanced growth rate on a target nitrogen source, host specific growth, modified sporulation characteristics or modified spores comprising: a flexible tubing wherein said tubing is subdivided by an operation of a gate into one or more discreet chambers, wherein one or more said gates are located in a fixed distance across longitudinal length of said tubing; one or more flywheels functionally connected to motors wherein said gate is mounted on the surface of said flywheel; a sampling port functionally connected with said flexible tubing wherein a sample of culture can be withdrawn through said sampling port; one or more inlets and outlets wherein said inlets and outlets allow air or culture media to be transported into said flexible tubing; and a timing device or a turbidimeter device wherein said device can instruct the movement of flywheel into user determined direction. In another embodiment, said device further comprises a thermoregulator. In one embodiment, said media has a temperature of about 48° C. In another embodiment, said media's temperature ranges from 44° C. to 49.7° C. In another embodiment, said media's temperature is incrementally increased from 44° C. to 49.7° C.

In another aspect, described herein is a thermotolerant strain of E. coli that can grow at a temperature of about 40° C. to about 70° C.

In another aspect, described herein is a thermotolerant strain of E. coli that can grow at a temperature of about 44° C. to about 49.7° C.

In another aspect, described herein is a thermotolerant strain of E. coli that can grow at a temperature of about 48° C.

In another aspect, described herein is a thermotolerant strain of E. coli that can grow at a temperature of about 48.5° C.

In another aspect, described herein is a thermotolerant strain of E. coli that has an increased doubling time at 37° C. than at 48° C.

In another aspect, described herein is a thermotolerant strain of E. coli comprising a mutation in the ylbE gene, kdpD gene, dgsA gene, rpoD gene, rpsJ gene, yhhZ gene, spoT gene, upstream of the yidE gene, treB gene, perR gene, malQ gene, wzzE gene, rpsA gene, pykF gene, proP gene, ybhN gene, yddB gene, pncB gene, mreD gene, malT gene, malS gene, upstream of the ppiC gene, rffT gene, glpF gene, upstream of the gltP gene, upstream of the yajD gene, fabA gene, upstream of the rydC gene, upstream of the yegT and fbaB gene, yejM gene, tktB gene, idi gene, or upstream of the yqjF gene. In another embodiment, said mutation is a frame shift, substitution, missense, point, translocation, insertion or deletion mutation. In another embodiment, said mutation is a point mutation.

In another aspect, described herein is a thermotolerant strain of M. anisopliae that can grow at a temperature of about 32° C. to about 40° C.

In another aspect, described herein is a thermotolerant strain of M. anisopliae that can grow at a temperature of about 37° C.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates directed evolution of thermotolerant M. anisopliae isolates.

FIG. 2 illustrates growth curves at 36.5° C. (A) and 37° C. (B) of wild-type and temperature adapted M. anisopliae isolates.

FIG. 3 illustrates differential interference contrast (DIC) images of wild-type and temperature adapted M. anisopliae strains grown at 37° C. EVG016 2 d (A) and 5 d (B), EVG017 2 d (C) and 5 d (D), Wild-type 2575 2 d (E) and 5 d (F). Bar=20 μm.

FIG. 4 illustrates infectivity and virulence of the wild type, EVG016 and EVG017g and other strains over 14 day period.

FIG. 5 illustrates growth of wild type and mutant E. coli strains on LB plates. 5A shows growth of MG1655 and EVG1064 at 30° C., 37° C. and 48.5° C. 5B shows growth of wild type and mutants at 30° C., 37° C., 43° C., 46° C., 48.5° C. and 49° C. 5C shows growth kinetics. The Tₘₐₓ for EVG1064 in liquid LB culture was 48.0° C. (FIG. 5C). Growth curves of WT and EVG1064 strains in liquid cultures at 37° C. and 48° C. Error bars indicate±1 std. deviation. FIG. 5D shows resistance to 30-minute exposures to elevated temperatures for WT and EVG1064.

FIG. 6 illustrates a continuous culture device.

FIG. 7 illustrates pulsed field gel electrophoresis of Xba1 digested genomic DNA from MG1655 and EVG1064. Lanes: (1) Lambda ladder (2) MG1655 (3) EVG1064 (4) Low range ladder (5) Mid range ladder (6) MG1655 (7) EVG1064 (8) Lambda ladder.

FIG. 8 illustrates mean generation times of MG1655 and EVG1064 plotted as a function of temperature. Error bars indicate±1 std. deviation.

### DESCRIPTION OF THE INVENTION

Methods, devices, and compositions described herein can artificially evolve a microorganism (natural, genetically engineered, or man-made) into a microorganism with one or more desired traits. A desired trait can be enhancement of natural characteristics of a microorganism or acquisition of one or more additional characteristics. An additional characteristic includes, but is not limited to, ability to control a pest, ability to adopt unnatural growth characteristics or life cycle, ability to grow in unnatural habitat, acquired tolerance to chemical, UV, or change in temperature tolerance. To artificially evolve a microorganism and to select for a desired trait any one of the continuous culture devices described herein can be used. Using methods, devices and compositions described herein, adaptation of an E. coli strain for growth in a higher than normal temperature range was achieved in about 8 months.

As used herein, the term “about” means the referenced numeric indication plus or minus 10% of that referenced numeric indication.

Continuous Culture Devices

Described herein is a continuous culture device. In one embodiment, the device cultures a microorganism continuously without having any wall growth problem. In another embodiment, the device evolves a microorganism by continuously culturing the microorganism and by having a selection means. In another embodiment, selection means is a physical culture condition. In another embodiment, physical culture condition is media. In another embodiment, physical culture condition is culture temperature, pH, light, pressure, or salinity. In another embodiment, physical culture condition is culture density. In another embodiment, physical culture condition is degree of dilution of the culture. In another embodiment, physical culture condition is an amount of radiation. In another embodiment, the evolutionary modification process uses a continuous culture method or apparatus described in U.S. patent application Ser. Nos. 11/508,286 or 10/590,348, which are herein incorporated by reference in their entirety. In another embodiment, a continuous culture device is used to produce an evolutionary modified microorganism (EMO) with one or more desired traits. In another embodiment, a contiuous culture device is a device described in example 1.

In one embodiment, an artificial evolutionary process performed by continuous culture devices described herein selects for certain traits. In another embodiment, selection is achieved by providing an evolutionary pressure. In another embodiment, evolutionary pressure is provided by pre-designed parameters. In another embodiment, a pre-designed parameter is one or more culture conditions. In another embodiment, arbitrary selection is provided by an assay system in which a strain exhibiting one or more desired traits is selected and repopulated in a continuous culture device.

In one embodiment, continuous culture device described herein is designed to achieve culturing a microorganism continuously without any fluid transfer, including sterilization or rinsing functions. In another embodiment, continuous culture is achieved inside a flexible sterile tube filled with growth medium. In another embodiment, the medium and the chamber surface are static with respect to each other, and both are regularly and simultaneously replaced by peristaltic movement of the tubing through “gates”, or points at which the tube is sterilely subdivided by clamps that prevent the cultured cells from moving between regions of the tube. UV gates can also (optionally) be added upstream and downstream of the culture vessel for additional security.

In one embodiment, continuous culture device can select continually, rather than periodically, against adherence of dilution-resistant variants to the chemostat surfaces, as replacement of the affected surfaces occurs in tandem with the process of dilution.

In one embodiment, the flexible sterile tube employed in continuous culture is subdivided in a transient way that there are regions containing saturated (fully grown) culture, fresh medium, and a region between these two. These transient, discrete regions form one or more chambers in which grown culture is mixed with fresh medium in a timely manner to continuously grow the culture. The gates are periodically released from one point on the tube and replaced at another point that grown culture along with its associated growth chamber surface and attached static cells is removed by isolation from the growth chamber and replaced by both fresh medium and fresh chamber surface.

In one embodiment, continuous culture proceeds by repetitive movements of the gated regions of tubing. This involves simultaneous movements of the gates, the tubing, the medium, and any culture within the tubing. In another embodiment, the tubing moves in the same direction; unused tubing containing fresh medium moves into the growth chamber and mixes with the culture remaining there, providing the substrate for further growth of the cells contained therein. Before being introduced into the growth chamber region, this medium and its associated tubing are maintained in a sterile condition by separation from the growth chamber by the upstream gates. Used tubing containing grown culture is simultaneously moved downstream and separated from the growth chamber by the downstream gates. As used herein, upstream refers to a portion of tubing containing fresh medium and downstream refers to a portion of tubing containing used medium.

In one embodiment, the boundaries between upstream chamber and the growth chamber or between the growth chamber and downstream chamber are defined by gates located along the tube. In another embodiment, gates are operated as clamps, either opening or closing off a section of tubing. In another embodiment, gates configurations, i.e., their locations, numbers, or the distance between gates, are adjusted according to species-specific demand of a culture. In a given configuration, gates can be designed through one chain of multiple teeth simultaneously moved or in another configuration separated moved in a distinctly synchronized manner. In another embodiment, gates comprise a system made of two teeth pinching the tubing.

In one embodiment, when one or more growth chambers are present, the growth chambers are used for the same or different purpose. For example, living cells can be grown in a first growth chamber and a second growth chamber with the same or different conditions. In another embodiment, a first growth chamber can be used to grow cells and a second growth chamber can be used to treat the living cells under different conditions. The cells can be treated to induce the expression of a desired product. Components or additives of the culture medium itself can be added prior to or after the culture begins. For example, all components or additives can be included in the media before beginning the culture, or components can be injected into one or more of the growth chambers after the culture have been initiated.

In one embodiment, aeration (gas exchange) is achieved by the use of gas permeable tubing. For example and without being limiting, flexible gas permeable tubing can be made of silicone. Aeration can be achieved through exchange with the ambient atmosphere or through exchange with an artificially defined atmosphere (liquid or gas) that contacts the growth chamber or enclosing the entire culture device. When an experiment demands anaerobiosis, the flexible tubing can be gas impermeable. For example, flexible gas impermeable tubing can be made of coated or treated silicone.

In one embodiment, anaerobic evolution conditions are achieved by confining regions of the tubing in a specific and controlled atmospheric area to control gas exchange dynamics. This is achieved either by making said thermostatically controlled box gastight and then injecting neutral gas into it or by placing the complete device in an atmosphere controlled room.

In one embodiment, the growing chamber is depressurized or over pressurized. Different ways of adjusting pressure can be used, for instance, by applying vacuum or pressurized air to the fresh medium and tubing through its upstream extremity and across the growth chamber. Another way of depressurizing or over pressurizing tubing can be done by alternate pinching and locking tubing upstream of or inside the growth chamber.

In one embodiment, continuous culture devices described herein use tilting movements of the device. In another embodiment, the devices use shaking movement. In another embodiment, cell aggregation is decreased and discouraged by shaking. In another embodiment, an external device is used for shaking. In another embodiment, one or several stirring bars are used in the tubing filled with fresh medium.

In one embodiment, continuous culture devices described herein use liquid or semi-solid material as a growth medium.

In one embodiment, continuous culture devices described herein contain multiple growth chambers. In another embodiment, multiple chambers are configured such that the downstream gates of one growth chamber become the upstream gates of another. In another embodiment, cells are allowed to grow alone in the first chamber, and then fed as the source of nutrition for a second cell in the second chamber.

In one embodiment, continuous culture devices described herein use an emitter to subject the cells, permanently or temporarily, to one or more of radio waves, light waves, UV-radiation, x-rays, sound waves, an electro magnetic field, a radioactive field, radioactive media, or combinations thereof. The growth chamber region of the device can be subjected to, permanently or temporarily, a different gravitational force. For example, the cells can be grown in a microgravity environment.

Methods and devices described herein are useful for adapting a strain to gain a trait including, but not limiting to, enhanced utilization of various nitrogen or carbohydrate sources, enhanced thermotolerance, enhanced cryotolerance, ultra-violet (UV)-light tolerance, enhanced growth rates, enhanced host specificity, enhanced chemical resistance, or modified sporulation. In one embodiment, the nitrogen and/or carbohydrate source is pieces of one ore more peset. In another embodiment, the nitrogen and/or carbohydrate source is insect debris. In another embodiment, an organism is evolved to obtain enhanced thermotolerance. In another embodiment, an organism is evolved to obtain enhanced cryotolerance. In another embodiment, an organism is evolved to obtain enhanced growth rate. In another embodiment, an organism is evolved to obtain UV-light tolerance. In another embodiment, an organism is evolved to obtain enhanced host specificity. In another embodiment, an organism is evolved to express the characteristics of enhanced chemical resistance. In another embodiment, an organism is evolved to express the characteristics of modified sporulation or modified spores. In another embodiment, the organism is an entomopathogenic fungus. In another embodiment, the fungus is a filamentous fungus. In another embodiment, the fungus is a M. anisopliae strain. In another embodiment, the filamentous fungus M. anisopliae strain 2575 is evolved to acquire thermotolerance (e.g., ability to grow) at 37° C. or higher. In another embodiment, the organism is a bacterium. In another embodiment, the bacterium is an E. coli. In another embodiment, the E. coli is E. coli K-12 MG1655.

Biocontrol Agent

In one embodiment, an EMO is used as a biocontrol agent. A biocontrol agent as used herein is a microorganism that is useful for controlling a pest. In another embodiment, a pest is an insect, a worm, a parasite, a snail, a slug, a mammal, a fish, a reptile or an amphibian. In another embodiment, an insect is grasshopper. In another embodiment, a snail is brown garden snail Cornu aspersum. In another embodiment, a snail is white garden snail, Theba pisana. In another embodiment, a slug is gray garden slug, Deroceras reticulatum. In another embodiment, a slug is tawny slug, Limacus flavus. In another embodiment, a biocontrol agent interferes with a pest's lifecycle. Interference includes, but is not limited to, reducing or suppressing the growth rate of a pest, killing a pest, increasing the growth rate of a natural predator of a pest, restraining the mobility of a pest, decreasing the fecundity of a pest, sterilizing a pest, creating unfavorable environment for a pest, exhausting a food source of a pest, or combinations thereof. A pest is any destructive insect or other animal that deteriorates the condition of crop, food, livestock, plant, wild animal, human, or building.

By employing methods, devices, and compositions described herein, a microorganism is evolved into a biocontrol agent or into a more effective biocontrol agent. In one embodiment, a biocontrol agent has pesticidal activity, such as insecticidal activity. In another embodiment, a biocontrol agent has enzymatic activity that interferes with a pest's lifecycle. In another embodiment, a microorganism has one or more biocontrol traits. In another embodiment, the biocontrol trait is naturally occurring. In another embodiment, the microorganism is artificially evolved to have a biocontrol trait. In another embodiment, a microorganism is artificially evolved to enhance an existing biocontrol trait. In another embodiment, methods and devices described herein improve a natural biocontrol trait of a microorganism. In another embodiment, methods and devices described herein evolve a microorganism to display a biocontrol trait not found in the wild type of the microorganism. In another embodiment, a microorganism that has a biocontrol trait is evolved to enhance the biocontrolling trait or to display another useful trait. In another embodiment, the useful trait is temperature adaptation. In another embodiment, in which a microorganism is evolved to display a robust growth in a climate different than the microorganism's natural habitat.

In one embodiment, a continuous culture device described herein is used to evolve a microorganism to display entomopathogenic activity. In another embodiment, a continuous culture device described herein is used to evolve a microorganism to enhance entomopathogenic activity. In another embodiment, the microorganism acquires enhanced ultraviolet (UV) light tolerance, enhanced growth rate, tropism toward unnatural host, chemical tolerance toward herbicide and/or insecticide, thermotolerance, cryotolerance, increased rate of target digestion, biological traits useful for containment, modified sporulation characteristics, or modified spores. In another embodiment, the microorganism is a bacterium, fungus, yeast, virus, algae, or any microorganism capable of sporulation.

Various entomophathogenic microorganisms can be used as a biocontrol agent. Entomophathogenic microorganisms include, but are not limited to, Adelges tsugae, Bemisia tabaci, Thrips tabaci, Hypothenemus hampei, Lymantria dispar, Hypera postica, Thrips tabaci, Pseudoplusia ni, Frankliniella occidentalis, Lymantria dispar, Solenopsis invicta, Paltothyreus tarsatus, Chironomus, Chironomus, Delphacodes kuscheli, Hypera postica, Eurygaster, Bemisia tabaci, Xiphinema americanum, Delia floralis, Meloidogyne hapla, Dialeurodes citri, Aglaia odoratissima, Dialeurodes citri, Trialeurodes vaporariorum, Dialeurodes citri, Dialeurodes citri, Dialeurodes citri, Megachile rotundata, Apis mellifera, Megachile, Apis mellifera, Megachile rotundata, Apis mellifera, Megachile, Megachile rotundata, Megachile centuncularis, Megachile rotundata, Chalicodoma, Ixodes scapularis, Supella longipalpa, Leptinotarsa decemlineata, Anthonomus grandis, Dolycorus, Nezara viridula, Eurygaster, Bemisia tabaci, Aeneolamia varia, Sogatella furcifera, Megachile rotundata, Rachiplusia nu, Plutella xylostella, Melanoplus, Myzus persicae, Anoplophora glabripennis, Pachnoda interrupta, Neobullieria citellivora, Anoplolepsis longipes, Bombyx mori, Phthorimaea operculella, Plutella xylostella, Galleria mellonella, Diaprepes abbreviata, Dolycorus, Eurygaster, Osmia lignaria, Nasutitermes acajutlae, Drosophila, Ixodes scapularis, Eurygaster, Lymantria dispar, Solenopsis invicta, Eoreuma loftini, Gorgonia ventalina, Phthorimaea operculella, Simulium vandalicum, Homo sapiens, Homo sapiens, Dendrolimus spectabilis, Acyrthosiphon pisum, Malacosoma disstria, Panolis flammea, Bradysia paupera, Acyrthosiphon kondoi, Acyrthosiphon pisum, Brevicoryne brassicae, Macrosiphum euphorbiae, Myzus ascalonicus, Myzus persicae, Rhopalosiphum maidis, Rhopalosiphum padi, Tipula paludosa, Empoasca fabae, Agrilus planipennis, Basilepta fulvicornis, Pachybrachis pallicornis, Coccinella septempunctata, Anthonomus grandis, Hypera postica, Shirahoshizo insidiosus, Anomala cuprea, Lachnosterna morosa, Popillia japonica, Xyloryctes jamaicensis, Tomicus minor, Tomicus pimperda, Tribolium castaneum, Eurygaster, Solenopsis invicta, Vespula vulgaris, Bombyx mori, Mocis, Spodoptera frugiperda, Chilo infuscatellus, Galleria mellonella, Cydia pomonella, Psacothea hilaris, Anomala costata, Popillia japonica, Nephotettix bipunctata cincticeps, Solenopsis, Ixodes scapularis, Varroa destructor, Anthicus floralis, Araecerus fasciculatus, Caryedon serratus, Agrilus planipennis, Amara familiaris, Amara plebeja, Bembidion lampros, Anoplophora glabripennis, Aromia moschata, Dectes texanus, Enaphalodes rufulus, Moechotypa diphysis, Monochamus alternatus, Monochamus scutellatus, Ortholeptura valida, Plectrodera scalator, Psacothea hilaris, Cerotoma, Cerotoma arcuata, Crimissa, Crimissa cruralis, Diabrotica, Diabrotica balteata, Diabrotica barberi, Diabrotica paranaensis, Diabrotica speciosa, Diabrotica undecimpunctata, Diabrotica virgifera, Galerucella sp., Galerucina, Leptinotarsa decemlineata, Lilioceris Maecolaspis monrosi, Notonata, Odontota dorsalis, Paropsis charybdis, Pyrrhalta luteola, Systena, Xanthogaleruca luteola, Coccinella, Coccinella septempunctata, Coleomegilla maculata, Cycloneda sanguinea, Hippodamia convergens, Ahasverus advena, Anthonomus grandis, Anthonomus musculus, Apion, Aracanthus, Ceutorhynchus litura, Chakodermus, Chalcodermus aeneus, Conotrachelus nenuphar, Cosmopolites, Cosmopolites sordidus, Curculio caryae, Curculio caryae, Cyrtepistomus castaneus, Diaprepes abbreviata, Geraeus senilis, Heilipodus erythropus, Hypera postica, Larinus, Listronotus oregonensis, Metamasius, Metamasius callizona, Metamasius hemipterus, Oryzophagus oryzae, Otiorhynchus ligustici, Otiorhynchus sulcatus, Phlyctinus callosus, Premnotryes latithorax, Premnotrypes suturicallus, Premnotrypes vorax, Rhynchites aequatus, Rhynchites baccus, Rhynchophorus ferrugineus, Sitona, Sitona discoideus, Sitona humeralis, Sitona lineatus, Sternechus subsignatus, Cylas formicarius elegantulus, Lagria vilosa, Cratomorphus diaphanus, Pytho, Rhizophagus grandis, Adoryphorus coulonii, Ancognatha scarabaeoides, Anomala cuprea, Anoplognathus, Aphodius tasmaniae, Costelytra zealandica, Pachnoda interrupta, Phyllophaga, Popillia japonica, Sericesthis nigrolineata, Dendroctonus ponderosae, Dryocoetes confusus, Hypothenemus hampei, Ips, Ips stenographus, Ips typographus, Tomicus minor, Anotylus rugosus, Anotylus sp., Gyrohypnus angustatus, Tachyporus sp, Alphitobius diaperinus, Pterohelaeus darlingensis, Tenebrio molitor, Sminthurus viridis, Alligator mississippiensis, Doru lineare, Forflcula africana, Forflcula auricularia, Delia antiqua, Delia radicum, Pegoplata aestiva, Calliphora, Scatella tenuicosta, Haematobia irritans, Musca autumnalis, Musca domestica, Phlebotomus papatasi, Tipula paludosa, Anthocoris nemorum, Leptoglossus fulvicornis, Blissus leucopterus, Nysius vinitor, Scolopostethus affinis, Mesovelia mulsanti, Adelphocoris, Leptopterna dolabrata, Liocoris tripustulatus, Lygus lineolaris, Lygus hesperus, Lygus lineolaris, Lygus pratensis, Notostira elongata, Stenodema laevigatum, Nabis, Acanthosoma labiduroides, Aelia, Dolycorus, Euschistus heros, Nezara viridula, Oebalus poecilus, Podisus, Tibraca limbativentres, Triatoma infestans, Leptocoris, Leptocoris oratorius, Eurygaster, Corythucha ciliata, Leptopharsa heveae, Lygus sp., Adelges tsugae, Bemisia tabaci, Trialeavrodes vaporariorum, Aphis gossypii, Diuraphis noxia, Myzus persicae, Rhopalosiphum padi, Schizaphis graminum, Deois flavopicta, Zulia carbonaria, Zulia entreriana, Balacha melanocephala, Molopopterus theae, Nephotettix bipunctata cincticeps, Nephotettix cincticeps, Pawiloma victima, Magicicada septendecim, Oliarus dimidiatus, Nilaparvata lugens, Kronides, Spissistilus festinus, Rhizoecus, Apis mellifera, Bombus, Cephaus, Diprion pini, Bephratelloides cubensis, Atta, Atta mexicana, Myrmica rubra, Pogonomyrmex occidentalis, Solenopsis, Solenopsis invicta, Solenopsis quinquecuspis, Solenopsis saevissima, Solenopsis xyloni, Pamphilius betulae, Lophyrotoma zonalis, Polistes, Coptotermes formosanus, Reticulitermes flavipes, Hyphantria cunea, Bombyx mori, Brassolis sophorea, Cossula cossus, Zeuzera pyrina, Danaus plexippus, Isturgia exerrariae, Oncopera, Oncopera alboguttata, Oncopera intricata, Hyblaea puer, Paraclemensia acerifoliella, Dendrolimus spectabilis, Malacosoma americanum, Lymantria dispar, Lymantria dissoluta, Leucoptera coffeella, Leucoptera scitella, Autographa gamma, Helicoverpa, Helicoverpa armigera, Helicoverpa virescens, Panolis flammea, Sesamia calamistis, Sesamia cretica, Simyra henrici, Spodoptera frugiperda, Spodoptera littoralis, Spodoptera litura, Carpocapsa pomonella, Emmalocera depressella, Plutella xylostella, Acigona sp, Chilo plejadellus, Cnaphalocrocis medinalis, Coniesta sp, Diatraea saccharalis, Dioryctria sylvestrella, Eldana saccharina, Galleria mellonella, Gymnancyla canella, Ostrinia nubilalis, Terastia meticulosalis, Opodiphthera eucalypti, Schirius, Monopetalotaxis doleriformis, Stenoma decora, Thaumetopoea pityocampa, Choristoneura, Cydia pomonella, Hedya nubiferana, Lobesia botrana, Rhyacionia frustrana, Yponomeutidae, Chrysopa, Austracris guttulosa, Locusta migratoria, Melanoplus, Melanoplus bivittatus, Oxyops vitiosa, Phaulacridium vittatum, Rhammatocerus schistocercoides, Schistocerca gregaria, Calliptamus italicus, Scapteriscus vicinus, Anabrus simplex, Homo sapiens, Haplothrips tritici, Frankliniella occidentalis, Thrips calcaratus, Trachemys scripta, Eurygaster, Adelg es tsugae, Diuraphis noxia, Solenopsis invicta, Eldana saccharina, Galleria mellonella, Anoplophora malasiaca, Niphonoclea, Anomala costata, Costelytra zealandica, Holotrichia parallela, Melolontha, Melolontha melolontha, Popillia japonica, Ips typographus, Eurygaster, Nephotettix bipunctata cincticeps, Castnia licus, Diatraea saccharalis, Galleria mellonella, Amphimallon solstitialis, Phoracantha semipunctata, Ixodes scapularis, Plutella xylostella, Armillaria mellea, Nilaparvata lugens, Hypothenemus hampei, Blattella germanica, Periplaneta americana, Wyeomyia smithii, Xiphinema, Tetranychus urticae, Brevicoryne brassicae, Orachrysops ariadne, Eotetranychus, Pemphigus betae, Thrips palmi, Aphis gossypii, Diaprepes abbreviata, Oncometopia tucumana, Sonesimia grossa, Simulium venustum, Orthezia praelonga, Hypera variabilis, Delia radicum, Ptychoptera contaminata, Tipula paludosa, Aphis fabae , Brevicoryne brassicae, Eriosoma lanigerum, Myzus persicae, Schizaphis graminum, Deois, Deois flavopicta, Nephotettix bipunctata cincticeps, Oliarus dimidiatus, Nilaparvata lugens, Sogatella furcifera, Gargara, Solenopsis invicta, Porcellio, Plutella xylostella, Choristoneura fumiferana, Homo sapiens, Volvariella volvacea, Ceutorhynchus napi, Lutzomyia, Lutzomyia sordelli, Acyrthosiphon pisum, Metopolophium dirhodum, Empoasca fabae, Nephotettix bipunctata cincticeps, Delphacodes haywardii, Nilaparvata lugens, Nasutitermes corniger, Mocis latipes, Plutella xylostella, Epinotia aporema, Homo sapiens, Frankliniella occidentalis, Plutella xylostella, Nilaparvata lugens, Nilaparvata lugens, Sogatella furcifera, Acyrthosiphon kondoi, Acyrthosiphon pisum, Aphis, Aphis armata, Macrosiphum euphorbiae, Metopolophium dirhodum, Rhopalosiphum maidis, Rhopalosiphum padi, Therioaphis maculata, Uroleucon, Hypera variabilis, Rhopalosiphum padi, Psila rosae, Culex pipiens pipiens, Acyrthosiphon pisum, Aphis fabae, Aphis glycines, Aphis gossypii, Cavariella theobaldi, Diuraphis noxia, Diuraphis tritici, Macrosiphum euphorbiae, Metopolophium dirhodum, Myzus persicae, Rhopalosiphum insertum, Schizaphis graminum, Therioaphis maculata, Uroleucon, Empoasca fabae, Sitophilus oryzae, Popillia japonica, Anomala cuprea, Forcipomyia marksae, Aedes kochi, Dasyhelea, Forcipomyia marksae, Aedes rupestris, Anopheles amictus hilli, Anopheles guadrimaculatus, Culiseta inconspicua, Culiseta inornata, Zulia carbonaria, Aelia, Diatraea saccharalis, Aeneolamia varia, Heterodera schachtii, Meloidogyne hapla, Elaphomyces, Elaphomyces, Pholcus phalangoides, Empoasca kraemeri, Nephotettix bipunctata cincticeps, Nilaparvata lugens, Parapodisma, Spilosoma niveum, Enypia griseata, Lambdina flscellaria flscellaria, Lambdina jiscellaria lugubrosa, Rheumaptera hastata, Dendrolimus spectabilis, Malacosoma disstria, Euproctis chrysorrhoea, Orgyia vetusta, Heliothis, Aedia leucomelas, Autographa gamma, Mamestra brassicae, Pseudaletia, Ellida caniplaga, Heterocampa, Heterocampa biundata, Heterocampa guttivitta, Colias erate poliographus, Dryocampa rubicunda, Choristoneura fumiferana, Cicadella, Empoasca kraemeri, Cicadetta puer, Nilaparvata lugens, Melanoplus bivittatus, Melanoplus cuneatus, Melanoplus differentialis, Melanoplus flavidus, Melanoplus packardii, Camnula pellucida, Dissosteira carolina, Malacosoma americanum, Malacosoma disstria, Lymantria dispar, Empoasca vitis, Praxibulus, Acyrthosiphon kondoi, Botanophila fugax, Delia, Delia antigua, Delia platura, Delia radicum, Pollenia rudis, Coenosia tigrina, Musca domestica, Ovatus crataegarius, Scatophaga stercoraria, Pollenia, Musca domestica, Psila rosae, Melanostoma scalare, Platycheirus clypeatus, Triglyphus primus, Thrips tabaci, Rhagonycha fulva, Hydrellia, Brevicoryne brassicae, Macrosiphum euphorbiae, Therioaphis maculata, Pseudoplusia includens, Plutella xylostella, Eana argentana, Aedes, Simulium, Tipula paludosa, Ptychoptera contaminata, Trachymyrmex sp., Acromyrmex octospinosus, Atta colombica, Agriotes, Phyllophaga menetriesi, Dendroctonus ruflpennis, Plecia, Chiromyza, Leptopharsa heveae, Pogonomyrmex occidentalis, Monophadnus elongatulus, Brassolis, Brassolis sophorea, Sitotroga cerealella, Spodoptera frugiperda, Anteotricha, Galleria mellonella, Agelastica alni, Procladius paludicola, Tanytarsus nr.inextentus, Malacosoma disstria, Pieris rapae, Notostira elongata, Boophilus, Tetranychus urticae, Agrilus planipennis, Lilioceris lilii, Anthonomus musculus, Chalcodermus aeneus, Conotrachelus nenuphar, Otiorhynchus ligustici, Sitona discoideus, Dendroctonus micans, Hypothenemus hampei, Musca domestica, Lutzomyia, Tetanops myopaeformis, Adelphocoris, Dolycorus, Triatoma infestans, Eurygaster, Adelges tsugae, Bemisia tabaci, Aphis fabae, Aphis gossypii, Diuraphis noxia, Pemphigus betae, Rhopalosiphum padi, Sitobion avenae, Toxoptera aurantii, Aeneolamia postica, Mahanarva andigena, Prosapia nr. bicincta, Zulia carbonaria, Zulia colombiana, Zulia pubescens, Coccus viridis, Nilaparvata lugens, Sogatella furcifera, Lopholeucaspis japonica, Ceresa bubalus, Heteropsylla cubana, Scrobipalpuloides absoluta, Hyblaea puer, Orachrysops subravus, Lymantria dispar, Spodoptera, Spodoptera frugiperda, Spodoptera litura, Emmalocera depressella, Plutella xylostella, Chilo sacchariphagus, Galleria mellonella, Cydia pomonella, Aiolopus longicornis, Tetrix granulata, Scirtothrips dorsalis, Meloidogyne hapla, Lymantria dispar, Lepidosaphes, Melanaspis obscura, Heteropsylla cubana, Lymantria dispar, Melanaspis glomerata, Nilaparvata lugens, Raghuva albipunctella, Delia radicum, Meloidogyne hapla, Zulia colombiana, Lymantria dispar, Nilaparvata lug ens, Heteropsylla incisa, Delia radicum, Pyrilla perpusilla, Pulvinaria elongata, Heteropsylla incisa, Chilo sacchariphagus indicus, Scirpophaga excerptalis, Lymantria dispar, Plutella xylostella, Adelges tsugae, Euophrys trivittata, Ixodes scapularis, Icerya purchasi, Aphelenchoides, Corynoneura, Meloidogyne hapla, Habrotrocha elusa, Abacarus hystrix, Sepedon sphegeus, Cyrtorhinus lividipennis, Empoasca kraemeri, Oliarus dimidiatus, Nilaparvata lugens, Lydda, Nuculaspis tsugae, Solenopsis invicta, Liothrips mikaniae, Taeniothrips inconseguens, Thrips palmi, Myndus crudus, Nilaparvata lugens, Diaphorina citri, Heteropsylla cubana, Ectopsocus, Heterocaecilius, Rastrococcus invadens, Brachyderes incanus, Empoasca kraemeri, Eriosoma lanigerum, Abacarus hystrix, Parthenolecanium corni, Choristoneura fumiferana, Heterodera glycines, Dioryctria zimmermani, Nilaparvata lugens, Criconemella curvata, Criconemella xenoplax, Heterodera glycines, Heterodera humuli, Heterodera schachtii, Dioryctria sylvestrella, Nephotettix virescens, Nilaparvata lugens, Calacarus heveae, Colomerus novahebridensis, Eriophyes guerreronis, Eriophyes sheldoni, Phyllocoptruta oleivora, Dolichotetranychus floridanus, Mononychellus tanajoa, Acalitus vaccinii, Idiocerus nitidulus, Idioscopus clypealis, Lymantria dispar, Trialeurodes vaporariorum, Lagria vilosa, Resseliella odai, Brachyderes incanus, Lymantria dissoluta, Agonum dorsale, Bembidion lampros, Hapalus sp, Anoplophora glabripennis, Pyrrhalta luteola, Otiorhynchus sulcatus, Premnotrypes vorax, Sitona lineatus, Lagria vilosa, Dendroctonus micans, Staphylinus olens, Alphitobius diaperinus, Tenebrio molitor, Aedes albifasciatus, Aedes sierrensis, Aelia, Eurygaster, Eurygaster integriceps, Adelges tsugae, Aleurocanthus woglumi, Bemisia tabaci, Trialeurodes vaporariorum, Diuraphis noxia, Acantholyda erythrocephala, Pristiphora erichsonii, Pyrrharctia isabella, Prionoxystus robiniae, Alosophila pometaria, Lambdina athasaria, Malacosoma americanum, Leucoma salicis, Lymantria dispar, Agrotis segetum, Rivula atimeta, Spodoptera, Quadicalcarifera punctatella, Chlosyne lacinia saundersii, Galleria mellonella, Ostrinia nubilalis, Conopia myopaeformis, Cydia pomonella, Laspeyresia medicagicus, Lobesia botrana, Taeniothrips inconseguens, Resseliella odai, Ixodes ricinus, Agrilus planipennis, Pyrrhalta luteola, Spaethiella, Lagria vilosa, Popillia japonica, Rhopaea magnicornis, Hypothenemus hampei, Alphitobius diaperinus, Tenebrio molitor, Blattella germanica, Calliphora, Musca autumnalis, Musca domestica, Adelphocoris, Bemisia, Bemisia argentifolii, Bemisia tabaci, Trialeurodes vaporariorum, Diuraphis noxia, Myzus persicae, Nilaparvata lugens, Phenacoccus solani, Heteropsylla incisa, Eretmocerus californicus, Hyphantria cunea, Bombyx mori, Lymantria dispar, Spodoptera, Plutella xylostella, Diaphania hyalinata, Galleria mellonella, Cydia pomonella, Litodactylus leucogaster, Bemisia, Trialeurodes vaporariorum, Mamestra brassicae, Bemisia argentifolii, Mogannia hebes, Mocis latipes, Spodoptera frugiperda, Agraulis vanillae, Xiphinema rivesi, Forcipomyia marksae, Aedes melanimon, Culex tarsalis, Culex territans, Culiseta melanura, Anoplolepsis longipes, Leptopharsa heveae, Adelges tsugae, Toxoptera citricida, Pogonomyrmex occidentalis, Taeniothrips inconsequens, Myzus nr. Persicae, Leptopharsa heveae, Entoloma, Myzus persicae, Musca domestica, Agrilus planipennis, Plectrodera scalator, Pyrrhalta luteola, Trialeurodes vaporariorum, Aphis rumicis, Brevicoryne brassicae, Diuraphis noxia, Myzus cerasi, Myzus persicae, Uroleucon ambrosiae, Ceroplastes, Coccus viridis, Lecanium viridis, Frankliniella occidentalis, Haematobia irritans, Trialeurodes vaporariorum, Diuraphis noxia, Cydia pomonella, Frankliniella occidentalis, Thrips tabaci, Tachyporus hypnorum, Trialeurodes vaporariorum, Macrosiphoniella sanborni, Myzus persicae, Rhopalosiphum nymphaeae, Toxoptera citricida, Agrilus planipennis, Hypera postica, Pissodes strobi, Malachius bipustulatus, Dendroctonus micans, Tenebrio molitor, Ochlerotatus triseriatus, Lutzomyia saulensis, Euryg aster, Trialeurodes vaporariorum, Aphis fabae, Diuraphis noxia, Macrosiphum euphorbiae, Myzus cerasi, Myzus persicae, Myzus nr. Persicae, Sitobion avenae, Ceroplastes, Coccus viridis, Parthenolecanium corni, Pulvinaria floccifera, Saissetia oleae, Delphacodes kuscheli, Cryptococcus fagisuga, Icerya purchase, Cossula cossus, Lymantria dispar, Galleria mellonella, Ostrinia nubilalis, Adoxophyes orana, Cydia pomonella, Frankliniella occidentalis, Hemileia vastatrix, Agrilus planipennis, Conotrachelus nenuphar, Cydia pomonella, Frankliniella occidentalis, Pissodes strobi, Raoiella indica, Lutzomyia sordelli, Euryg aster, Bemisia tabaci, Sitobion avenae, Icerya aegyptica, Rhizoecus, Meloidogyne hapla, Olivea colebrookeae, Xiphinema, Aedes aegypti, Aedes albifasciatus, Culex, Culex pipiens quinquefasciatus, Mansonia titillans, Cruznema lambdiense, Iragoides fasciata, Taeniothrips inconsequens, Magicicada septendecim, Fiorinia externa, Dermolepida albohirtum, Rhopaea magnicornis, Galleria mellonella, Calliptamus italicus, Zonocerus variegates, Austracris guttulosa, Locusta migratoria capito, Ornithacris cavroisi, Patanga succincta, Schistocerca piceifrons, Kraussaria angulifera, Zonocerus elegans, Cofana spectra, Nephotettix virescens, Recilia dorsalis, Austracris guttulosa, Kraussaria angulifera, Zonocerus variegatus, Eumerus strigatus, Dermolepida albohirtum, Lepidiota consobrina, Oryctes rhinoceros, Xyloryctes jamaicensis, Spodoptera, Boophilus, Haphochelus marginalis, Agrianome spinicollis, Anoplophora glabripennis, Brontispa longissima, Cerotoma arcuata, Diabrotica, Diabrotica speciosa, Coleomegilla maculate, Blosyrus asellus, Chalcodermus aeneus, Curculio caryae, Desiantha diversipes, Geraeus senilis, Otiorhynchus ligustici, Otiorhynchus sulcatus, Rhabdoscelus obscurus, Sternechus subsignatus, Agriotes, Agriotes sputator, Conoderus, Limonius canus, Adoryphorus coulonii, Anomola, Anoplognathus, Anoplognathus hirsutus, Antitrogus consanguineus, Antitrogus mussoni, Antitrogus parvulus, Aphodius tasmaniae, Costelytra zealandica, Cyclocephala, Dasygnathus dejeani, Dermolepida albohirtum, Heteronychus arator, Heteronyx, Heteronyx piceus, Heteronyx rugosipennis, Lepidiota consobrina, Lepidiota frenchi, Lepidiota gibbifrons, Lepidiota negatoria, Lepidiota noxia, Lepidiota picticollis, Lepidiota squamulata, Melolontha melolontha, Oryctes, Pachnoda interrupta, Papuana, Phyllopertha horticola, Phyllophaga anxia, Phyllophaga anxia, Popillia japonica, Rhopaea magnicornis, Rhopaea verreauxii, Sericesthis micans, Sericesthis nigrolineata, Sericethis, Alphitobius diaperinus, Tenebrio molitor, Tribolium castaneum, Delia floralis, Ochlerotatus triseriatus, Hydrellia, Scatella tenuicosta, Boreoides tasmaniensis, Inopus rubriceps, Nezara viridula, Scotinophara coarctata, Tibraca limbativentres, Diuraphis noxia, Pemphigus trehernei, Aeneolamia varia, Deois, Deois flavopicta, Deois incomplete, Kanaima fluvialis, Mahanarva posticata, Mahanarva sp., Zulia carbonaria, Zulia colombiana, Zulia pubescens, Recilia dorsalis, Nilaparvata lugens, Anagyrus, Atta, Myrmica rubra, Myrmica scabrinodis, Cryptotermes brevis, Neotermes, Mastotermes, Mastotermes darwiniensis, Coptotermes, Coptotermes acinaciformis, Coptotermes formosanus, Coptotermes frenchi, Coptotermes lacteus, Drepanotermes perniger, Microcerotermes, Nasutitermes exitiosus, Oncopera alboguttata, Oncopera intrucata, Wiseana sp., Malacosoma disstria, Spodoptera, Spodoptera frugiperda, Chlosyne lacinia saundersii, Plutella xylostella, Diatraea saccharalis, Eoreuma loftini, Galleria mellonella, Acrotylus, Oxya multidentata, Phaulacridium vittatum, Schistocerca gregaria, Schistocerca piceifrons, Calliptamus italicus, Teleogryllus commodus, Homo sapiens, Brontispa longissima, Otiorhynchus sulcatus, Conoderus, Heteronyx piceus, Phyllophaga cuyabana, Aeneolamia varia, Deois flavopicta, Mahanarva flmbriolata, Mahanarva posticata, Zulia pubescens, Nephotettix virescens, Nilaparvata lugens, Mastotermes darwiniensis, Coptotermes lacteus, Helicoverpa zea, Mocis, Eoreuma loftini, Ostrinia nubilalis, Schistocerca gregaria, Boophilus, Anoplophora glabripennis, Otiorhynchus sulcatus, Sitona lineatus, Agriotes, Aphodius tasmaniae, Diloboderus abderus, Melolontha melolontha, Phyllopertha horticola, Popillia japonica, Tenebrio molitor, Tribolium castaneum, Aedes crinifer, Ochlerotatus triseriatus, Nilaparvata lugens, Solenopsis invicta, Coptotermes formosanus, Bombyx mori, Oxycanus, Leucoptera scitella, Anticarsia gemmatalis, Spodoptera frugiperda, Carpocapsa pomonella, Galleria mellonella, Lobesia botrana, Schistocerca piceifrons, Otiorhynchus sulcatus, Lachnosterna bidentata, Chortoicetes terminifera, Otiorhynchus sulcatus, Adoryphorus coulonii, Nephotettix virescens, Recilia dorsalis, Nilaparvata lugens, Pemphigus, Pemphigus trehernei, Adoryphorus, Coptotermes lacteus, Pyrausta machaeralis, Myllocerus discolor, Sitona discoideus, Melolontha melolontha, Papuana woodlarkiana, Bombyx mori, Pseudosphingonotus savignyi, Dermolepida albohirtum, Lepidiota consobrina, Anoplognathus, Oryctes, Oryctes rhinoceros, Bombyx mori, Zygogramma bicolorata, Diaprepes abbreviata, Antitrogus mussoni, Antitrogus parvulus, Costelytra zealandica, Oryctes rhinoceros, Scapanes australis, Scaptores castanea, Scotinophara coarctata, Nephotettix cincticeps, Nephotettix virescens, Nilaparvata lugens, Cryptotermes brevis, Coptotermes lacteus, Galactica, Spodoptera, Phaulacridium vittatum, Pseudosphingonotus savignyi, Ornebius kanetataki, Teleogryllus commodus, Dectes texanus, Cerotoma arcuata, Diabrotica, Curculio caryae, Listronotus oregonensis, Otiorhynchus sulcatus, Conoderus, Ancognatha scarabaeoides, Heteronychus arator, Heteronyx, Phyllophaga anxia, Popillia japonica, Rhizotrogus majalis, Strigoderma arboricola, Tribolium castaneum, Cirtonemus, Atta sexdens rubropilosa, Solenopsis, Kalotermes, Chlosyne lacinia saundersii, Plutella xylostella, Galleria mellonella, Oncopera intricata, Oncopera intrucata, Pyrrhalta fuscipennis, Holotrichia parallela, Bemisia tabaci, Trialeurodes vaporariorum, Corcyra cephalonica, Meloidogyne hapla, Meloidogyne hapla, Lymantria dispar, Lymantria dispar, Anoplophora glabripennis, Zulia vilior costarricensis, Fiorinia externa, Porcellio, Nasutitermes acajutlae, Bunonema, Bertia moriformis, Tetranychus urticae, Mononychellus tanajoa, Tetranychus urticae, Thrips tabaci, Tetranychus althaeae, Helicoverpa armigera, Nephila clavipes, Pomponia linearis, Sogatella furcifera, Bombyx mori, Lymantria, Alabama argillacea, Anticarsia gemmatalis, Helicoverpa armigera, Mocis frugalis, Naranga, Plathypena scabra, Plusia, Plusiinae, Prodenia litura, Pseudoplusia includens, Rachiplusia nu, Rivula atimeta, Spodoptera, Spodoptera exigua, Spodoptera frugiperda, Spodoptera litura, Cnaphalocrocis medinalis, Cryptotympana facialis, Aphodius howitti, Macrotermes, Ciadetta puer, Xylophagus sp., Taxus sp., Nezara. viridula, Adelges tsugae, Bemisia argentifolii, Bemisia tabaci, Recilia dorsalis, Diaphorina citri, Nasutitermes acajutlae, Natada michonta, Sesamia inferens, Cydia pomonella, Epiphyas postvittana, Heptophylla picea, Gastropacha orientalis, Zulia carbonaria, Cerotoma, Diaprepes abbreviata, Lagria vilosa, Hypothenemus hampei, Tenebrio molitor, Scotinophara coarctata, Tibraca limbativentres, Triatoma infestans, Eurygaster, Trialeurodes vaporariorum, Diuraphis noxia, Deois flavopicta, Zavlia pubescens, Anoplolepsis longipes, Pogonomyrmex occidentalis, Nasutitermes corniger, Bombyx mori, Chlosyne lacinia saundersii, Opsiphanes cassinae, Plutella xylostella, Galleria mellonella, Meloidogyne, Zulia carbonaria, Plutella xylostella, Athalia rosae, Plutella, Plutella maculipennis, Plutella xylostella, Empoasca fabae, Nephotettix cincticeps, Recilia dorsalis, Nilaparvata lugens, Sogatella furcifera, Sogatodes pusanus, Spissistilus festinus, Mamestra brassicae, Acyrthosiphon kondoi, Lygus, Acyrthosiphon pisum, Aphidula, Aphis, Aphis fabae, Aphis glycines, Brevicoryne brassicae, Dactynotus formosanus, Diuraphis noxia, Hyalopterus pruni, Hyperomyzus lactucae, Macrosiphum, Macrosiphum akebiae, Macrosiphum euphorbiae, Macrosiphum rosae, Metopolophium dirhodum, Microlophium carnosum, Myzus, Myzus nicotinae, Myzus persicae, Schizaphis graminum, Sitobion, Sitobion avenae, Uroleucon formosanus, Acyrthosiphon kondoi, Lipaphis erysimi, Therioaphis maculata, Antitrogus rugulosus, Ixodes scapularis, Tetranychus urticae, Anthonomus musculus, Chalcodermus bimaculatus, Drosophila, Zulia colombiana, Orthezia praelonga, Vespula germanica, Oncopera alboguttata, Lymantria dispar, Hieroglyphus banian, Camponotus, Lymantria dispar, Lymantria dispar, Lymantria dispar, Anthonomus musculus, Lagria vilosa, Lymantria dispar, Hemitrichia serpula, Heterodera glycines, Habrotrocha elusa, Elaphe, Elaphe obsoleta, Crotalus horridus, Anoplophora glabripennis, Leptinotarsa decemlineata, Lucilia illustris, Adelges tsugae, Bemisia tabaci, Lymantria dispar, Plutella xylostella, Bemisia tabaci, Leptopharsa heveae, Orthocladius, Polypedilum, Psectrocladius limbatellus, Pseudokiefferiella, Tanytarsus, Tanytarsus, Cricotopus, Orthocladius, Microtendipes, Chironomus, Corynoneura, Tanytarsus, Aedes albifasciatus, Aedes sticticus, Culex, Culex pervigilans, Culex renatoi, Prosimulium, Simulium vittatum, Austrothaumalea, Dactylolabis montana, Limonia, Dasyhelea, Chironomus alternans, Orthocladius, Aedes albopictus, Aedes crinifer, Aedes vexans, Culex, Culex dolosus, Culex restuans, Culiseta, Culiseta impatiens, Culiseta incidens, Ochlerotatus japonicus, Simulium vittatum, Cricotopus, Chironomus, Psectrocladius, Dicrotendipes fumidus, Simulium, Chironomus, Simulium vittatum, Orthocladius, Ochlerotatus triseriatus, Psectrocladius sordidellus, Diamesa, Chironomus, Aphrophila bidentata, Diamesa, Simulium, Simulium uchidai, Cricotopus, Elliptera astigmatica, Paraheptagyia, Melanoplus, Scapteriscus vicinus, Nilaparvata lug ens, Paltothyreus tarsatus, Sitobion avenae, Aphelenchoides, Adoryphorus coulonii, Scotinophara coarctata, Adelges tsugae, Bemisia tabaci, Brevicoryne brassicae, Prosapia plagiata, Lymantria dispar, Artipes, Delia radicum, Tetanops myopaeformis, Tetanops myopaeformis, Promecotheca papuana, Nilaparvata lugens, Leptopharsa heveae, Delia floralis, Plecia nearctica, Aedes australis, Aedes sierrensis, Myrmica rubra, Sirex noctilo, Arachnocampa luminosa, Mycobates, Choristoneura fumiferana, Nilaparvata lugens, Euophrys trivittata, Empoasca kraemeri, Unaspis citri, Anoplolepsis longipes, Elasmopalpus lignosellus, Adelges tsugae, Aeneolamia varia, Adelphocoris, Nephotettix bipunctata cincticeps, Adelphocoris, Lymantria dispar, Aelia, Eurygaster, Trialeurodes vaporariorum, Myzus persicae, Icerya purchasi, Taeniothrips inconsequens, Hemileia vastatrix, Otiorhynchus sulcatus, Agelastica alni, Otiorhynchus sulcatus, Sitona lineatus, Carcinops pumilio, Aphodius flmetarius, Dendroctonus micans, Alphitobius diaperinus, Sminthurus viridis, Delia radicum, Scatella stagnalis, Musca domestica, Eurygaster, Leptopharsa heveae, Adelges tsugae, Dreyfusia normannianiae, Bemisia tabaci, Trialeurodes vaporariorum, Aphis gossypii, Brachycaudus helichrysi, Brevicoryne brassicae, Diuraphis noxia, Macrosiphoniella sanborni, Myzus persicae, Sitobion avenae, Toxoptera citricida, Coccus hesperidium, Coccus viridis, Phytokermes hemichryphus, Pulvinaria aurantii, Cryptococcus fagisug a, Formica sp., Bombyx mori, Thrips tabaci, Puccinia striiformis, Bambusaspis sp., Forflcula auricularia, Trechus quadristriatus, Agriotes sputator, Notostira elongata, Anoecia corni, Acyrthosiphon pisum, Macrosiphum euphorbiae, Brevicoryne brassicae, Myzus rannaculinum, Hypera postica, Hypera punctata, Hypera variabilis, Delia radicum, Dicyphus pallidus, Aphis fabae, Brachycaudus amygdalinus, Capitophorus, Diuraphis noxia, Drepanosiphum aceris, Metopolophium dirhodum, Therioaphis maculata, Therioaphis trifolii f maculata, Empoasca, Empoasca fabae, Empoasca kraemeri, Empoasca vitis, Hauptida distinguenda, Molopopterus theae, Typhlocyba, Delphacodes striatella, Nilaparvata lugens, Psyllida etrioza, Trioza urticae, Neodiprion tsugae, Tuta absoluta, Anacampsis humilis, Lambdina flscellaria flscellaria, Sesamia inferens, Trichoplusia ni, Pieris brassicae, Plutella xylostella, Cnaphalocrocis medinalis, Choristoneura fumiferana, Epinotia aporema, Merophyas divulsana, Ptycholoma aeriferana, or Tortrix viridian.

In one embodiment, an evolutionarily modified microorganism (EMO) described herein can control pests in crops such as corn, wheat, millet, triticale, soybean, teff, fonio, buckwheat, quinoa, common bean, chickpea, lima bean, runner bean, pigeon, garden pea, lupin, maize, oats, barley, rye, rice or sorghum; in fruit, for example stone fruit, pome fruit and soft fruit such as apples, pears, plums, peaches, almonds, cherries or berries, for example strawberries, raspberries and blackberries; in legumes such as beans, lentils, peas or soya beans; in oil crops such as oilseed rape, mustard, poppies, olives, sunflowers, coconuts, castor-oil plants, cacao or peanuts; in the marrow family such as pumpkins, cucumbers or melons; in fiber plants such as cotton, flax, or jute; in citrus fruit such as oranges, lemons, grapefruit or tangerines; in vegetables such as spinach, lettuce, asparagus, cabbage species, carrots, onions, tomatoes, potatoes, beet or capsicum; in the laurel family such as avocado, cinnamon or camphor; in tobacco, nuts, coffee, egg plants, sugar cane, tea, pepper, grapevines, hops, the banana family, latex plants or ornamentals, tomatoes, cotton, potatoes, sugar beet.

In one embodiment, strains evolved by methods, devices, and compositions described herein are also useful for protecting one or more species of a plant, such as a tree, a fruit bearing plant, a vegetable, a horticultural plant or other agricultural crop. In another embodiment, strains evolved by methods, devices, and compositions described herein are also useful for protecting one or more species of tree, such as deciduous trees, evergreen trees, coniferous trees. Trees include, but are not limited to, an ash tree, a beech tree, a birch tree, a maple tree, an oak tree, a pine tree or a willow tree. In another embodiment, strains evolved by methods, devices, and compositions described herein are also useful for protecting one or more species of fruit-bearing plants. Fruit bearing plants include, but are not limited to, grape vines, strawberry plants, an apple tree, a pear tree, a plum tree, a citrus tree (e.g., lemon, lime, orange or grapefruit) or other fruit trees. In another embodiment, strains evolved by methods, devices, and compositions described herein are also useful for protecting one or more species of vegetable plants. Vegetable plants include, but are not limited to, tomatoes, cucumbers, carrots, green beans, celery, peas, broccoli, asparagus, cauliflower, water chestnuts, lettuce varietals, onions, garlic, cabbage, melons, pumpkins, or watermelons. In another embodiment, strains evolved by methods, devices, and compositions described herein are also useful for protecting one or more species of agricultural crops such as cotton, wheat, corn, rice, soybean, sorghum, or sugar cane. In one embodiment the agricultural crop is a monoculture crop.

In one embodiment, strains evolved by methods, devices, and compositions described herein are also useful for protecting economically important horticultural plants. Examples of horticultural plants include, but are not limited to greenhouse plants, nursery plants or ornamental plants not grown in a field. In one embodiment, an ornamental plant is a rose, minirose, carnation, tulip, herb, rhododendron, magnolia, primrose, orchid, chrysanthemum or poinsettia. In another embodiment, a greenhouse plant is a greenhouse vegetable grown year-round, such as tomato, onion, green onion, or potato. In another embodiment, a greenhouse plant is an ornamental plant. In another embodiment, a greenhouse plant is a plant grown from a seed.

In one embodiment, an evolved microorganism is used to protect an economically important crops, such as corn. In another embodiment, an evolved microorganism is used to protect soybean. In another embodiment, an evolved microorganism is used to protect a potato.

In one embodiment, an EMO described herein can be used to control one or more species of insect. In another embodiment, the EMO kills the insect. In another embodiment, the EMO interferes with an insect's ability to reproduce. Insects as contemplated herein refer to an adult insect or any developmental stages thereof, such as nymphs or larvae. Insects that can be effectively controlled by methods, devices, and compositions described herein include, but are not limited to, the order Lepidoptera, such as armyworms, cutworms, loopers, and heliothines in the family Noctuidae (e.g., fall armyworm (Spodoptera fugiperda J. E. Smith), beet armyworm (Spodoptera exigua Hubner), black cutworm (Agrotis Ipsilon Hufnagel), cabbage looper (Trichoplusia ni Hubner), tobacco budworm (Heliothis virescens Fabricius)); borers, casebearers, webworms, coneworms, cabbageworms and skeletonizers from the family Pyralidae (e.g., European corn borer (Ostrinia nubilalis Hubner), navel orangeworm (Amyelois transitella Walker), corn root webworm (Crambus caliginosellus Clemens), sod webworm (Herpetogramina licarsisalis Walker)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck)); other economically important lepidoptera (e.g., diamondback moth (Plutella xylostella Linnaeus), pink bollworm (Pectinophora gossypiella Saunders), gypsy moth (Lymantria dispar Linnaeus)); the order Blattodea including cockroaches from the families Blattellidae and Blattidae (e.g., oriental cockroach (Blatta orientalis Linnaeus), Asian cockroach (Blatella asahinai Mizukubo), German cockroach (Blattella gennanzica Linnaeus), brownbanded cockroach (Supella longipalpa Fabricius), American cockroach (Periplanieta americana Linnaeus), brown cockroach (Periplaizeta brunnea Burmeister), Madeira cockroach (Leucophaea maderae Fabricius)); the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scaribaeidae (e.g., Japanese beetle (Popillia japonica Newman) and European chafer (Rhizotrogus majalis Razoumowsky)); carpet beetles from the family Dermestidae; wireworms from the family Elateridae; bark beetles from the family Scolytidae and flour beetles from the family Tenebrionidae; the order Dermaptera including earwigs from the family Forficulidae (e.g., European earwig (Forficula auricularia Linnaeus), black earwig (Chelisoches mono Fabricius)); the orders Hemiptera and Homoptera such as, plant bugs from the family Miridae, cicadas from the family Cicadidae, leafhoppers (e.g. Empoasca spp.) from the family Cicadellidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, cinch bugs (e.g., Blissus spp.) and other seed bugs from the family Lygaeidae, spittlebugs from the family Cercopidae squash bugs from the family Coreidae, red bugs and cotton stainers from the family Pyrrhocoridae; the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)), flat mites in the family Tenuipalpidae (e.g., citrus flat mite (Brevipalpus lewisi McGregor)), rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae (e.g., deer tick (Ixodes scapularis Say), Australian paralysis tick (Ixodes holocyclus Neumann), American dog tick (Dermacentor variabilis Say), lone star tick (Amblyomma americanum Linnaeus) and scab and itch mites in the families Psoroptidae, Pyemotidae, and Sarcoptidae; the order Orthoptera including grasshoppers, locusts and crickets (e.g., migratory grasshoppers (e.g., Melanoplus sanguinipes Pabricius, M. differentialis Thomas), American grasshoppers (e.g., Schistocerca americana Drury), desert locust (Schistocerca gregaria Forskal), migratory locust (Locusta migratoria Linnaeus), house cricket (Acheta domesticus Linnaeus), mole crickets (Gryllotalpa spp.)); the order Diptera including leafminers, midges, fruit flies (Tephritidae), frit flies (e.g., Oscinella frit Linnaeus), soil maggots, house flies (e.g., Musca doinestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phonnia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; the order Thysanoptera including onion thrips (Thrips tabaci Lindeman) and other foliar feeding thrips; the order Hymenoptera including ants (e.g., carpenter ant, red carpenter ant (Camponotus ferrugineus Pabricius), black carpenter ant (Camponotus pennsylvanicus De Geer), Pharaoh ant (Monomorium pharaonis Linnaeus), little fire ant (Wasmannia auropunctata Roger), fire ant (Solenopsis geminata Fabricius), red fire ant, red imported fire ant (Solenopsis invicta Buren), Argentine ant (Iridomyrmex humilis Canr), crazy ant (Paratrechina longicornis Latreille), pavement ant (Tetramorium caespitum Linnaeus), cornfield ant (Lasius alienus Forster), odorous house ant (Tapinoma sessile Say)), bees (including carpenter bees), hornets, yellow jackets and wasps; the order Isoptera including the eastern subterranean termite (Reticulitermes flavipes Kollar), western subterranean termite (Reticuliternes hesperus Banks), Formosan subterranean termite (Coptotermes formosanus Shiraki), West Indian drywood termite (Incisitermes immigrans Snyder) and other termites of economic importance; the order Thysanura such as silverfish (Lepisma saccharina Linnaeus) and firebrat (Thermobia domestica Packard); the order Mallophaga and including the head louse (Pediculus humanus capitis De Geer), body louse (Pediculus humanus humanus Linnaeus), chicken body louse (Menacanthus strainineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests include, but are not limited to, spiders in the order Araneae such as the brown recluse spider (Loxosceles reclusa Gertsch & Mulaik) and the black widow spider (Latrodectus mactans Fabricius), centipedes in the order Scutigeromorpha such as the house centipede (Scutigera coleoptrata Linnaeus); the order Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenee (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hubner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermuller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworn), Spodoptera litura Fabricius (tobacco cutworm, cluster caterpillar), Spodoptera frugiperda J. E. Smith (fall armyworm), Trichoplusia ni Hubner (cabbage looper) and Tuta absoluta Meyrick (tomato leafminer); the order Homoptera including: Acyrthisiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip aphid), Metopolophium dirrhodum Walker (cereal aphid), Macrosipum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (nglish grain aphid), Therioaphis maculata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stal (rice leafhopper), Nilaparvata lugens Stal (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erythroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla); the order Hemiptera including: Acrosternuin hilare Say (green stink bug), Anasa tristis De Geer (squash bug), Blissus leucopterus leucopterus Say (chinch bug), Corythuca gossypii Fabricius (cotton lace bug), Cyrtopeltis modesta Distant (tomato bug), Dysdercus suturellus Herrich-Schaffer (cotton stainer), Euchistus servus Say (brown stink bug), Euchistus variolarius Palisot de Beauvois (one-spotted stink bug), Graptosthetus spp. (complex of seed bugs), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper); Thysanoptera (e.g., Frankliniella occidentalis Pergande (western flower thrip), Scirthothrips citri Moulton (citrus thrip), Sericothrips variabilis Beach (soybean thrip), and Thrips tabaci Lindeman (onion thrip); and the order Coleoptera (e.g., Leptinotarsa decemlineata Say (Colorado potato beetle), Epilachna varivestis Mulsant (Mexican bean beetle) and wireworms of the genera Agriotes, Athous or Limonius.

In one embodiment, an EMO is useful for controlling worms. The term worm includes an adult form, as well as other forms of a worm's developmental stage, such as a nymph, or a larva stage. An EMO can target one of or all developmental stages of a worm for controlled reduction. Worms that can be controlled by methods, devices, and compositions described herein include, but are not limiting to, members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus); animal and human health pests such as flukes, tapeworms, and roundworms, such as Strongylus vulgaris in horses, Toxocara canis in dogs, Haemonchus contortus in sheep, Dirofilaria immitis Leidy in dogs, Anoplocephala perfoliata in horses, and Fasciola hepatica Linnaeus in ruminants.

Filamentous fungi are among the most widely used whole cell biocatalysts in a host of agricultural, food, environmental and bioenergy related applications. Fungi have complex regulatory circuits that intimately control cellular growth and metabolism. Continuous culture methods described herein can select for genetic variants that exhibit desired traits.

Many fungal species are known to cause infections in insects or mites. These are generally known as entomopathogenic fungi. These species attack a wide range of insect and mite species. In one embodiment the fungi produce spores that infect their host by germinating on its surface and then growing into its body. Once inside the body, the fungi multiply, causing the death of host insect. The fungi produce new spores in the dead body, which then are dispersed and repeat the cycle by germinating on new hosts. Thus, an infected host or an insect can be a medium for the dispersion of the fungi. One example of entomophathogenic process is described in Hajek et al (“Pathology and Epizootiology of Entomophaga maimaiga infections in Forest Lepidoptera, Microbiol Mol. Biol. Rev. 63:814-835, 1999), which is incorporated herein by reference in its entirety.

In one embodiment, an entomopathogenic fungus can be used as a bioinsecticide. Entomopathogenic fungi include, but are not limited to, strains in the class of Hyphomycetes. Hyphomycetes are virulent against insects and act by forming stable infective conidia upon contact with insects. In another embodiment, an effective entomopathogenic fungus is lethal for target insects but less harmful for non-target insects.

An insect cuticle is an exoskeleton serving as an interface between the insect and environment. It is an important element of an insect defense against a variety of external factors such as mechanical stress, dry, wet, cold or hot environment. The insect cuticle participates in diverse epidermal secretions, stores chemicals, and serves as a structural part of mechanoreceptors or chemoreceptors. The cuticle comprises chitin, epidermal cells and other secreted proteins. A cuticle is subdivided into epicuticle and procuticle. In one embodiment each cuticle layer has several sub-layers. In addition, there are two layers comprising the epidermis containing epidermal cells producing the cuticle and a basal membrane supporting the epidermal cells. In one embodiment, Beauveria bassiana initiates infection by a germinating spore (conidium) attached to an insect cuticle. The attachment leads to penetration of the cuticle of insect host. As the fungus penetrates the target pest cuticle, the invasive hyphae begin to enter the host tissues and branch out through the hemocoel. Hyphal bodies or segments of the hyphae are formed throughout the hemocoel, filling the insect with mycelium. At this point, the insect begins to die. Hyphal growth emerges out through the insect's body and spores are produced on the external surface of the host. These spores, or conidia, are airborne and capable of infecting new host.

In one embodiment, the biological cycle of B. bassiana includes two phases, a pathogenic phase and a saprophytic phase. Pathogenesis is manifested when the fungus comes into contact with live tissues of the host. Infection occurs through conidia. At first, a conidium is germinated, which is followed by a penetration and development of hyphae inside the insect. This process takes 3 to 4 days. In another embodiment, penetration of an insect cuticle is achieved by B. bassiana via enzymatic secretions such as lipases, chitinases and proteases. Passing through the cuticle layer, conidial germ tubes penetrate soft intersegmental membrane of the insect and begin to extend hyphae into the sect, establishing infection site upon which the killing process is ensued. At the end of the sporulation, which is the beginning of a new cycle, fungal mycelium can be observed in the soft parts of the insect.

In one embodiment, methods and devices described herein are used to evolve strains of B. Bassiana. Strains of B. Bassiana include, but are not limited to, strains of B. bassiana (Balsamo) Vuillemin or isolates of B. bassiana. Certain strains of B. bassiana produce high concentrations of stable conidia that produce morbidity in three to ten days. For example, Beauveria bassiana Bb05002 NRRL 30976 is virulent against Varroa mites, but has limited effects on honeybee hives or colonies. In another embodiment, a virulent strain of B. bassiana is a species specific strain.

In one embodiment, methods and devices described herein are used to evolve one or more strains of Metarhizium. Strains of Metarhizium include, but are not limited to, strains of M. anisopliae, M. flavoviridae, M. majus, or M. acridum. Certain strains Metarhizium is known for and has been used for locust control, producing high amounts of spores that can germinate on live insect upon contacting the insect's cuticle.

Lethality of bioinsecticide can be expressed as LT50, which is the time that takes to kill 50% of the target insect population at a given dose under a particular environmental condition. LT50 can be expressed in the number of hours or days to kill half of the target population. Under experimentally controlled environment, LT50 can be recorded as the time taken to kill half of the target population at a specified temperature, humidity, or both. Conidia are asexual spores, which can be counted and used as units of measure of the fungus, for example, with respect to viability and LT50. In another embodiment, a microorganism is evolved to acquire a shorter LT50 than that of the wild type. In another embodiment, methods and devices described herein artificially evolutionary modify a microorganism to shorten its natural LT50 by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days. In another embodiment, methods and devices described herein artificially evolutionary modify a microorganism to shorten its natural LT50 by by about between 1 and 3 days, between 3 and 6 days, between 6 and 9 days, between 9 and 12 days, between 1 and 4 days, between 3 and 7 days, between 6 and 10 days, between 9 and 13 days, between 1 and 5 days, between 3 and 8 days, between 6 and 11 days, between 9 and 14 days, between 1 and 6 days, between 3 and 9 days, between 6 and 12 days, between 9 and 15 days, between 1 and 7 days, between 3 and 10 days, between 6 and 13 days, between 9 and 16 days, between 1 and 8 days, between 3 and 11 days, between 6 and 14 days, between 9 and 17 days, between 1 and 9 days, between 3 and 12 days, between 6 and 15 days, between 9 and 18 days, between days, between 1 and 4 days, between 2 and 4 days, between 2 and 5 days, between 2 and 6 days, between 2 and 7 days, between 2 and 8 days, between 3 and 10 days, between 3 and 6 days, between 3 and 7 days, between 3 and 8 days, between 3 and 9 days, between 4 and 10 days, between 4 and 11 days, between 4 and 7 days, between 4 and 8 days, or between 4 and 9 days.

UV-Tolerance

In one embodiment, a microorganism is artifically evolutionarily modified to increase its tolerance to ultra violet light (UV light). In another embodiment, the microorganism is a bacterium, virus, algae, fungus, or a microorganism capable of sporulation. In another embodiment, the microorganism is a bacterium. In another embodiment, the bacterium is a strain of E. coli. In another embodiment, a wild type microorganism is artifically evolutionarily modified to tolerate a range of UV light unfavorable for the growth or survival of the wild type. In another embodiment, the microorganism is artifically evolutionarily modified to become tolerant to a range of wavelengths of UV light either above or below the natural UV range in which the microorganism grows. In another embodiment, the microorganism is artifically evolutionarily modified to become tolerant to a specific wavelength of UV light either above or below the natural UV range in which the microorganism grows. In another embodiment, a candidate microorganism for developing the trait of enhanced UV tolerance is selected based on having other useful traits, such as targeting a particular host, insecticidal activity, or chemical production.

In one embodiment, a microorganism is artifically evolutionarily modified by being continuously cultured in the presence of UV light. In another embodiment, the duration of UV light emission is controlled by a timing device or turbidity device. In another embodiment, a microorganism adopted a tolerance to a particular UV light wavelength or target UV range emerges from a continuous culture by outgrowing non-evolved microorganism.

In one embodiment, a microorganism acquires enhanced UV light tolerance. In another embodiment, the microorganism is continuously cultured in the presence of one or more wavelengths of UV-light. In another embodiment, a microorganism is artifically evolutionarily modified by exposure to a range of wavelengths of ultraviolet radiation including, but is not limited to, 10-121 nm, 10-150 nm, 88-100 nm, 10-200 nm, 122-200 nm, 100-280 nm, 200-300 nm, 280-315 nm, 300-400 nm, or 315-400 nm. In another embodiment, a microorganism is artifically evolutionarily modified by exposure to about 10 nm, 11 nm, 12 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, or 400 nm. In another embodiment, a microorganism is evolved to grow under sunlight.

The UV-light sources contemplated herein include, but are not limited to, artificial or natural source (such as the sunlight). In one embodiment, a UV-light source is a UV fluorescent lamp, a UV light-emitting diode, a UV laser, or a gas-discharge lamp (e.g., argon, neon, krypton, xenon). In another embodiment, a UV-light source is sunlight. In another embodiment, the sunlight is filtered or limited to a certain wavelength or a range of wavelengths by a light filter, a beam polarizer, a narrow band filter, or a filter for a specific wavelength or certain ranges of wavelengths. In another embodiment, a UV lamp is FischerBiotech™ 15 w UV lamp. In another embodiment, a UV lamp is Spectroline™ short-wavelength UV lamp. In another embodiment, a UV lamp is UV-C irradiator (Thermo Scientific™).

In one embodiment, UV light exposure is intermittent during continuous culture. In another embodiment, intermittent UV exposure is accomplished by providing a shutter device operably connected to a timing device. In another embodiment, UV light exposure is continuous during continuous culture. In another embodiment, continuous exposure is timed for a pre-determined period. The total amount of energy imparted on to the culture via UV light can be experimentally determined and adjusted depending on the rate of adaptation (e.g., survival rate). Examples of the total amount of energy delivered by UV light include, but are not limited to, about 5, 10, 20, 30, 50, 80, 100, 150, 200, 250, 300, 350, 400, 450, 500, 1250, 2000, 3000, 5000, 7500, 10,000, 15,000, 20,000, 25,000, 30,000, 35000, 40,000, 45,000, 50,000, 55,000, 60,000, 65,000, 70,000, 75,000, 80,000, 85,000, 90,000, 95,000, and 100,000 Joules/m2. Examples of the total amount of energy delivered by UV light also include, but are not limited to, about 5, 10, 20, 30, 50, 80, 100, 150, 200, 250, 300, 350, 400, 450, 500, 1250, 2000, 3000, 5000, 7500, 10,000, 15,000, 20,000, 25,000, 30,000, 35000, 40,000, 45,000, 50,000, 55,000, 60,000, 65,000, 70,000, 75,000, 80,000, 85,000, 90,000, 95,000, and 100,000 Joules/cm2. Examples of the total amount of energy delivered by UV light ranges from about 1-5, 10-20, 30-50, 80-100, 150-200, 250-300, 350-400, 450-500, 1250-2000, 3000-5000, 7500-10,000, 15,000-20,000, 25,000-30,000, 35000-40,000, 45,000-50,000, 55,000-60,000, 65,000-70,000, 75,000-80,000, 85,000-90,000, or 95,000-100,000 Joules/m2. Alternatively, the total amount of energy delivered by UV light includes, but is not limited to, about 5-10, 20-30, 50-80, 100-150, 200-250, 300-350, 400-450, 500-1250, 2000-3000, 5000-7500, 10,000-15,000, 20,000-25,000, 30,000-35000, 40,000-45,000, 50,000-55,000, 60,000-65,000, 70,000-75,000, 80,000-85,000, 90,000-95,000, or 100,000 Joules/m2. Examples of the total amount of energy delivered by UV light can range from about 1-5, 10-20, 30-50, 80-100, 150-200, 250-300, 350-400, 450-500, 1250-2000, 3000-5000, 7500-10,000, 15,000-20,000, 25,000-30,000, 35000-40,000, 45,000-50,000, 55,000-60,000, 65,000-70,000, 75,000-80,000, 85,000-90,000, and 95,000-100,000 Joules/cm2. Alternatively, examples of the total amount of energy delivered by UV light also include, but are not limited to, about 5-10, 20-30, 50-80, 100-150, 200-250, 300-350, 400-450, 500-1250, 2000-3000, 5000-7500, 10,000-15,000, 20,000-25,000, 30,000-35000, 40,000-45,000, 50,000-55,000, 60,000-65,000, 70,000-75,000, 80,000-85,000, 90,000-95,000, and 100,000 Joules/cm2. In another embodiment, a UV light is delivered to a microorganism in short-burst with an energy level or with a range of energy levels described herein. In another embodiment, a UV light is delivered to an organism for a long-term with an energy level or with a range of energy levels described herein. In another embodiment, the organism is exposed to a UV light for a defined period of time, which is opttionally repeated at intervals. In another embodiment, UV light is delivered to a microorganism for about 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, 60 sec, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, 60 min, 2 hour, 3 hour, 4 hour, 5 hour, 6 hour, 7 hour, 8 hour, 9 hour, 10 hour, 11 hour, 12 hour, 13 hour, 14 hour, 15 hour, 16 hour, 17 hour, 18 hour, 19 hour, 20 hour, 21 hour, 22 hour, 23 hour, 24 hour, 2 day, 3 day, 4 day, 5 day, 6 day, 7 day, 8 day, 9 day, 10 day, 11 day, 12 day, 13 day, 14 day, 15 day, 16 day, 17 day, 18 day, 19 day, 20 day, 21 day, 22 day, 23 day, 24 day, 25 day, 26 day, 27 day, 28 day, 29 day, 30 day, 31 day, 2 month, 3 month, 4 month, 5 month, 6 month, 7 month, 8 month, 9 month, 10 month, 11 month, 12 month, 2 year, 3 year, 4 year, 5 year, 6 year, 7 year, 8 year, 9 year, 10 year, 11 year, 12 year, 13 year, 14 year, 15 year, 16 year, 17 year, 18 year, 19 year, or 20 year.

In one embodiment, a fungal strain is artifically evolutionarily modified by exposure to UV-light, then drying the exposed fungal strain, collecting the resulting spores and optionally exposing the spores to UV-light. In another embodiment, spores are stored for a period of time and placed in continuous culture device while being exposed to UV light. In another embodiment, spores are exposed to UV light of certain wavelength and intensity that is different than what is used for the continuous culture.

In one embodiment, a bacterial strain is is artifically evolutionarily modified by exposure to UV-light, then storing the bacterial strain in a cryopreservative medium known in the art (e.g., 10% glycerol mixed with culture medium). In another embodiment, the bacterial strain is stored for a period of time and placed in continuous culture and re-exposed to UV light. In another embodiment, a bacterial strain is exposed to UV light of certain wavelength and intensity that is different than what is used for the continuous culture.

In one embodiment, to artificially evolve a microorganism, various media compositions are employed in continuous culture. Suitable culture media are known in the art. Examples of media known to those skilled in the art and which are commercially available include media containing potato, dextrose, agar, or rice agar. In another embodiment, the media is a fungal culture media. In another embodiment, the fungal culture media comprises about 1% dextrose, about 1% yeast extract, about 5% rice flour, about 1.5% agar and about 0.5% 5× Dubois sporulation salts. In another embodiment, a fungal culture media comprises about 0.3-4% by weight of malt extract (preferably 0.5-3%, and most favorably 2%), about 0.3-4% by weight of yeast extract (preferably 0.5-3%, and most favorably 2%), about 0.1-2% by weight of peptone (preferably 0.3-1%, and most favorably 0.5%), about 1-5% by weight of glucose (preferably 2-4%, and most favorably 2%), about 30-70% by weight of water (preferably 40-60%, and most favorably 50%), about 30-70% by weight of solid base (preferably 40-60%, and most favorably 50%), and about 0.3-4% by weight of calcium carbonate or gypsum (preferably 0.5-3%, and most favorably 2%). In another embodiment, a microorganism is continuously cultured with commercially available media, such as Sabouraud dextrose (SAB) media. In another embodiment, a microorganism is continuous cultured with debris of a host insect. In another embodiment, the debris comprises fragments of whole host insects. In another embodiment, a medium comprises carbon source, nitrogen source, trace elements, vitamins, organic compounds, and inorganic compounds. In one embodiment continuous culture lasts for about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, 7 months, 8 months, 9 months, 10 months, 11 months, 12 months, 13 months, 14 months, 15 months, 16 months, 17 months 18 months, 19 months, 20 months, 21 months, 22 months, 23 months or 2 years.

Acquisition of UV-tolerance can be experimentally confirmed by measuring proximal parameters to UV tolerance. In another embodiment, UV-tolerance is measured by growth rate (e.g., rate of cell division and/or rate of sporulation) in the presence of the UV light that the strain is evolved to. The growth rate can be measured over a period of time. The time period can be hours, days, weeks, or months. Growth rates of evolved strains are graphed over a period and used as guidance for selecting and classifying evolved strains for their longevity under a particular UV wavelength. In terms of longevity, evolved strains can be classified as short-living, e.g., days to weeks to a few months, or as long-living, e.g., 6 months, a year or longer. A short-living strain is useful for short-term treatment of pest insects. An example of short-term treatment is seasonal treatment. A short-living strain is useful for applications where containment after the use of artificially evolved strains is difficult. For example, in windy area where dispersion of spore is likely to affect agricultural area not intended for treatment, short-living strains can be preferable. A long-living strain is useful for application against non-seasonal or year-round pest insects. In another embodiment, a short-living strain can be remedial for an infestation. In another embodiment, a long-living strain can be preventive of an anticipated infestation.

Growth Rate

In one embodiment, an a microorganism is is artifically evolutionarily modified to have a faster growth rate than an unmodified microorganism. In another embodiment, the microorganism is a bacterium, virus, algae, fungus, or a microorganism capable of sporulation. In another embodiment, the bacterium is an E. coli strain. In another embodiment, a microorganism is evolutionarily modified to acquire a growth rate faster than that of the wild type microorganism. In another embodiment, the microorganism is evolutionarily modified to grow faster on a specific carbon or nitrogen source. In another embodiment, the microorganism is evolutionarily modified to grow faster on a host insect. In another embodiment, the evolutionary modification involves continuously culturing a microorganism on debris of a host insect species. In another embodiment, a microorganism evolved for rapid growth is a bacterium.

Growth rate of a culture can be measured by methods widely used in microorganism culture. In one embodiment, growth rate is measured by cell counting and charting the number of cells over a period of time. In another embodiment, a small sample is taken regularly from a growing culture for a period of time and the number of cells is counted in a cell counter. A counter can be a manual counter or an automatic counter. In another embodiment, a manual counter is a hemocytometer. In another embodiment, an automatic counter is a Coulter™ counter. In another embodiment, cell counting can be assisted by cell staining to easily visualize the counted cell. For a bacterial cell counting, for example, any dye that interacts with bacterial cell wall can be used. In another embodiment, the dye is acridine orange. Sampling time depends on the types of evolved organism. In another embodiment, a sample can be taken every 1-2 hours up to every 3-4 days. In another embodiment, a sampling can be performed in every hour for a week. In another embodiment, sampling can be performed every half an hour for about 3-days to one week. In another embodiment, sampling can be performed every day for the length of time the microorganism is cultured. In another embodiment, growth rate is measured by optical density (O.D.). In another embodiment, change of optical density is charted over a period of time and growth rate is obtained by calculating the slope of the graph. In another embodiment, growth rate is obtained by calculating the time it takes for a microorganism population to double in density. In another embodiment, a light emitter at 595 nm is used to measure the optical density or a culture. In another embodiment, turbidity of a culture is used as a proxy measure for the optical density of a culture. In another embodiment, a UV/Visible spectrophotometer is used to measure optical density. In another embodiment, a Beckman™ UV/Visible spectrophotometer is used to measure the optical density.

In one embodiment, rapid growth of an EMO is beneficial for an application of an EMO as a bioinsecticide because it reduces the LT50. In another embodiment, a microorganism is evolved to reach a rapid growth rate in which less than 0.1%, 0.5%, 0.8%, 1.0%, 5%, or 10% of the intended protected target population (e.g., industrial crop or animal) is damaged upon the application of the evolved microorganism. In another embodiment, a microorganism is evolved to reach a growth rate that would prevent the target pest from reaching a reproductive stage. In another embodiment, rapid growth rate is adopted to shorten time for expansion at the application site. For example, rapid growth rate is helpful for controlling large coverage area in short time. In another embodiment, rapid growth rate is adopted to reduce the amount of start culture required to maintain the strain in storage. In another embodiment, rapid growth rate is adopted to reduce transportation cost of the stock microorganism from the manufacturing site to the site of application. Under an environmental condition where death of a large percentage of a wild type strain is expected, a microorganism adapted for rapid growth can compensate for the rate of death and thus maintain a level of presence higher than that of a wild type strain. Rapid growth rate can also be economical. For example, because of its rapid expansion, the size of initial spray zone can be smaller than that of wild type strain. A spray zone can be an agricultural field, a residence, a park, a farm or a building. An intended target of protection includes, but is not limited to, crop, forest, structure, a body of water such as a river or a lake, a wild animal, a farm animal or a human. A farm animal includes, but is not limited to, dog, cat, chicken, goose, pig, alpaca, bison, camel, cattle, deer, donkey, horse, goat, llama, mule, rabbit, reindeer, sheep, water buffalo, or yak.

In one embodiment, a bacterial or fungal species is artifically evolutionarily modified to acquire a faster growth rate. In another embodiment, a bacterial or fungal species is placed in a continuous culture device described herein to evolve a faster growth rate. In another embodiment, a different ratio of dilution is applied to cultured strain while it is being continuously cultured. By continuously applying dilution to strains emerging in the culture, a selection pressure is applied to the culture in which a group of fastest growing strains is passed to the next round of dilution while slower growing strains are eliminated. The rate of growth can be tested by methods known in the art. For example, growth rate of a strain can be measured by optical density of a sample of evolving microorganism.

In one embodiment, a fast growing strain is selected by adjusting parameters of a continuous culture device described herein. For example, modifying the rate of advancement of culture tubing favors the survival of faster growing strain.

The rate of dilution applicable for evolving a strain to acquire faster growing rates can be strain specific. In general, the dilution can be as low as 1:1,000,000 to as high as 1:5 (volume to volume) between a stock of strain prepared from exponentially growing culture (O.D. 0.4-0.8) and a sample medium containing no culture. In one embodiment, the dilution is about 1:750,000. In another embodiment, the dilution is about 1:500,000. In another embodiment, the dilution is about 1:250,000. In another embodiment, the dilution is about 1:100000. In another embodiment, the dilution is about 1:75000. In another embodiment, the dilution is about 1:50000. In another embodiment, the dilution is about 1:25000. In another embodiment, the dilution is about 1:10000. In another embodiment, the dilution is about 1:7500. In another embodiment, the dilution is about 1:5000. In another embodiment, the dilution is about 1:2500. In another embodiment, the dilution is about 1:1000. In another embodiment, the dilution is about 1:750. In another embodiment, the dilution is about 1:500. In another embodiment, the dilution is about 1:250. In another embodiment, the dilution is about 1:100. In another embodiment, the dilution is about 1:75. In another embodiment, the dilution is about 1:50. In another embodiment, the dilution is about 1:25. In another embodiment, the dilution is about 1:10. In another embodiment, the dilution is about 1:8. In another embodiment, the dilution is about 1:5.

Other types of selection pressure can be applied to a microorganism in order to acquire faster growth rate. In one embodiment, a fungus is grown in gaseous atmosphere containing chemically inert gas. In another embodiment, helium is applied as a selection pressure. Depending on the types of microorganism and a particular evolutionary condition, other gases can be applied. For example, a particular mix of carbon dioxide and oxygen can be used. In another embodiment, the mixture can be about 5% oxygen, 10% oxygen, 15% oxygen, 20% oxygen or higher. In another embodiment, the content of carbon dioxide in a mix can be about 1%, 2%, 5%, 10%, 15%, 20%, or higher. In another embodiment, a mixture can be a mix of natural air with an inert gas. In another embodiment, a mixture can be a mix of two types of gas, such as oxygen and carbon dioxide. In another embodiment, the gas can be nitrogen.

Limiting certain gas component, such as oxygen or carbon dioxide, can also be introduced into continuous culture as an added pressure to select for a faster growing strain. Varying the salt concentration of a medium (e.g., change of salinity of media) can also be introduced into continuous culture. In one embodiment, salinity is less than about 0.05%. In another embodiment, the salinity is between about 0.05% and 3%. In another embodiment, the salinity is between about 3% and 5%. In another embodiment, the salinity is more than about 5%. These selection pressures can be present continuously or applied intermittently throughout the selection process. Two or more of these selection pressures can be applied in combinations, concomitantly, tandemly, alternatively, or cyclically.

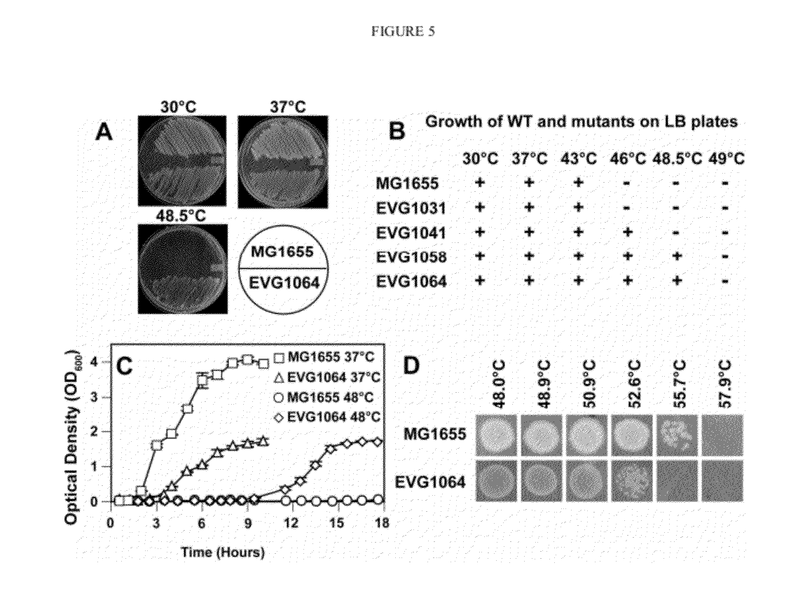

In one embodiment, a microorganism is artifically evolutionarily modified to acquire a faster growth rate by which the microorganism's LT50 is 3 days from the time of application. In another embodiment, the microorganism's LT50 is 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 2 days, or 1 day from the time of application. In another embodiment, the microorganism's LT50 is about one week, two weeks, three weeks, four weeks, five weeks, six weeks, seven weeks, eight weeks, nine weeks or ten weeks from the time of application. In another embodiment, a microorganism is evolved to acquire a faster growth rate by which the microorganism's LT50 is 2 days from the time of application. In another embodiment, a microorganism is evolved to acquire a faster growth rate by which the microorganism's LT50 is 1 day from the time of application. In another embodiment, a microorganism shown in FIG. 5 is selected as a starting microorganism and evolved to acquire a LT50 of 3 days. In another embodiment, a microorganism is evolved to shorten LT50 by 1, 2, 3, 4, 5, 6, 7, 8, 9, or 10 days from the microorganism's natural LT50.

Target Specificity

In one embodiment, methods, devices, and compositions described herein are used to artificially evolutionarily modify a microorganism to acquire target specificity (e.g., a pest or a part of a pest). In another embodiment, the microorganism is a bacterium, virus, algae, fungus, or a microorganism capable of sporulation. In another embodiment, a microorganism evolved for target specificity is a bacterium. In another embodiment, the bacterium is an E. coli strain.

In one embodiment, a microorganism is grown in the presence of substrate (e.g. food source) prepared from the target pest. In another embodiment, the substrate comprises a specific carbon source. In another embodiment, the substrate comprises a specific nitrogen source. In another embodiment, a bacterial strain is evolved to grow on substrate prepared from a single type of insect. In another embodiment, a bacterial strain is evolved to grow on substrate prepared from two or more different types of insects.

In one embodiment, a microorganism is artificially evolutionarily modified for growth and germination on one type of insect but not on a closely related species. To do so, insect extracts are prepared by using natural material obtained from the insect. For example, insects are washed in an ethanol bath and then quickly frozen in liquid nitrogen. The frozen insects are then fractured by applying physical force upon them. Fractured insects debris can be used either directly or processed further before being fed to a microbial strain.

To test for target species selectivity or cross-reactivity on other strains, a strain or species growing robustly on an insect extract is tested on another insect extract obtained from a closely related species. For testing a large number of targets, a library of insect extracts can be prepared in a small scale and applied to a high throughput, short-term culture platforms known in the art. In another embodiment, a microorganism continuously cultured on one type of insect extract is interrogated by a high throughput culture system for target specificity. In another embodiment, insect extracts are prepared from bees and wasps by freeze-fracturing methods described above. A bacterial strain growing robustly on wasp extract is tested for growth on bee extract. In another embodiment, bacterial strains or species evolved to grow on wasp extract, but not on bee extract, are selected as a biocontrol agent.

In one embodiment, target species specificity is catalogued by the identity and the number of the target insects a microorganism can effectively control. In another embodiment, the microorganism is a bacterial strain. In another embodiment, the bacterial strain targets more than one insect species. In another embodiment, the bacterial strain targets a single insect species. In another embodiment, a bacterial strain kills members of a single insect species without harming members of another insect species. In another embodiment, a bacterial strain kills members of two or more insect species without harming members of another insect species. By screening for growth on extracts from closely related insects, a bacterial strain can be evolved for targeting single insect species, a group of closed related species, or a genus. In another embodiment, a bacterial strain is evolved to kill members of a genus of insect species.

In one embodiment, a microorganism is evolutionarily modified for enhanced target specificity by increasing genetic diversity in the culture being evolved. In another embodiment, genetic diversity is increased by culturing cells with one or more agents increasing genetic mutation. In another embodiment, one or more agents that increase genetic mutation are chemical mutagens, irradiation, micro RNA, or other methods of causing mutations in the genome. These mutational agents can be introduced to the culture at the beginning of continuous culture to increase the diversity of genetic pool. In another embodiment, mutational agents are used in addition to other evolutionary modification methods described herein. In another embodiment a mutational agent is used while an organisms is also exposed to UV light (either periodically, continuously or once). In another embodiment, a mutational agent is used while an organism is selected for temperature adaptation such as thermotolerance or cryotolerance.

In one embodiment, M. anisopliae is evolved to acquire target specificity. In another embodiment, M. flavoviridae is evolved to acquire target specificity. In another embodiment, B. bassiana is evolved to acquire target specificity. In another embodiment, a strain of M. anisopliae is cultured in a continuous culture device described herein. In another embodiment, a culture medium includes biological material obtained from an insect cuticle. In another embodiment, the biological material is an extract. In another embodiment, the extract is produced by physically or chemically treating an insect. In another embodiment, a physical treatment such as freeze-thawing is used. In another embodiment, a frozen cuticle is fractured by physical force. In another embodiment, carbohydrate and protein are extracted from insect cuticle. In another embodiment, extraction utilizes enzymes such as proteinase K. In another embodiment, extraction utilized denaturing buffer such as guanidine HCl. In another embodiment, extraction utilizes chemical such as alcohol. In another embodiment, whole unprocessed cuticle is used for culture. In another embodiment, culture medium includes biological material obtained from worms.

In one embodiment, M. anisopliae is grown on a beetle cuticle. In another embodiment, B. bassiana is grown on ant cuticle. Other targets of B. Bassiana include, but are not limited to, aphids, whiteflies, mealybugs, psyllids such as lygus bugs or chinch bug, grasshoppers, fillips, termites, fire ants, flies, stem borers such as fungal gnats or shoreflies, beetles such as coffee borer beetle, colorado potato beetle, mexican bean beetle, japanese beetle, boll weevil, cereal leaf beetle, bark beetles, black vine weevil, or strawberry root weevil, caterpillars, such as european corn borer, codling moth, douglas fir tussock moth, or silkworm, and mites.

Rapid Pesticide Activity

In one embodiment, a microorganism is artificially evolutionarily modified to rapidly colonizing a target pest, such as an insect. In another embodiment, the target pest is an insect. In another embodiment, the microorganism is a bacterium, virus, algae, fungus, or a microorganism capable of sporulation. In another embodiment, the microorganism is a bacterium. In another embodiment, the bacterium is an E. coli strain. In another embodiment, a microorganism is evolved to rapidly colonize a target pest that is a fungus.

In one embodiment, a bacterium, fungus, or a microorganism capable of sporulation can be artificially evolutionarily modified to rapidly colonizing a target pest. In another embodiment, a bacterial strain is placed with insect cuticles in a continuous culture device described herein. After a period of culture, the rate of germination, colonization, and spore formations are measured as indicia for the rapidity of insecticidal activity. Alternatively, insect extract prepared from target insect's cuticle can be used. Insect extract can be produced by freeze-fracturing method described herein or by grinding, dissolving, heating, or a chemical treatment known in the art. In another embodiment, a bacterial strain evolved to acquire target specificity is further evolved to acquire rapid colonization of the substrate.

Chemical Tolerance

In one embodiment, a microorganism is artificially evolutionarily modified to acquire tolerance to a chemical. In another embodiment, the microorganism is a bacterium, virus, algae, fungus, or a microorganism capable of sporulation. In another embodiment, the microorganism is a bacterium. In another embodiment, the bacterium is an E. coli strain. In another embodiment, the chemical inhibits the growth or reproduction of wild-type microorganism.

In one embodiment, the chemical is herbicide, insecticide or a fungicide. By acquiring compatibility with widely used insecticide or herbicide, a microorganism can be applied on a field already treated with herbicide or insecticide. A microorganism can be remedial in situations where food or energy crop has been treated with chemical herbicide or insecticide but the treatment fails to control the infestation. Compatibility also helps in which a microorganism provides a long-term protection against pests while chemical treatment provides short-term remedy to infestation.

In one embodiment, a microorganism described herein is cultured in the presence of chemical in a continuous culture device described herein. In another embodiment, the chemical is herbicide, insecticide or a fungicide. In another embodiment, the initial concentration of herbicide or insecticide included in the culture is empirically determined. In another embodiment, a microorganism is cultured with a gradually increasing concentration of a chemical. Initial concentration of a chemical can be as low as 1/1,000,000 of lethal dose that kills 50% (LD50) of the treated microorganism population. In another embodiment, the initial concentration of a chemical is 1/1,000,000 of LD50. In another embodiment, the initial concentration of a chemical is about 1 ppm. Other examples of starting concentrations include, but are not limited to, about 2 ppm, 3 ppm, 5 ppm, 7 ppm, 8.5 ppm, 10.2 ppm, 11.9 ppm, 13.6 ppm, 15.3 ppm, 17 ppm, 18.7 ppm, 20.4 ppm, 22.1 ppm, 23.8 ppm, 25.5 ppm, 27.2 ppm, 28.9 ppm, 30.6 ppm, 32.3 ppm, 34 ppm, 35.7 ppm, 37.4 ppm, 39.1 ppm, 40.8 ppm, 42.5 ppm, 44.2 ppm, 45.9 ppm, 47.6 ppm, 49.3 ppm, or 51 ppm. In another embodiment, the starting concentration can be about 50 ppm, 70 ppm, 100 ppm, 123 ppm, 148 ppm, 173 ppm, 198 ppm, 223 ppm, 248 ppm, 273 ppm, 298 ppm, 323 ppm, 348 ppm, 373 ppm, 398 ppm, 423 ppm, 448 ppm, 473 ppm, 498 ppm, 523 ppm, 548 ppm, 573 ppm, 598 ppm, 623 ppm, 648 ppm, 673 ppm, 698 ppm, 723 ppm, 748 ppm, 773 ppm, 798 ppm, 823 ppm, 848 ppm, 873 ppm, 898 ppm, 923 ppm, 948 ppm, 973 ppm, or 998 ppm. In another embodiment, the initial concentration of a chemical is about 1 uM, 3 uM, 6 uM, 9 uM, 11.5 uM, 14.2 uM, 16.9 uM, 19.6 uM, 22.3 uM, 25 uM, 27.7 uM, 30.4 uM, 33.1 uM, 35.8 uM, 38.5 uM, 41.2 uM, 43.9 uM, 46.6 uM, 49.3 uM, 52 uM, 54.7 uM, 57.4 uM, 60.1 uM, 62.8 uM, 65.5 uM, 68.2 uM, 70.9 uM, 73.6 uM, 76.3 uM, 79 uM, 81.7 uM, 84.4 uM, 87.1 uM, 89.8 uM, 92.5 uM, 95.2 uM, 97.9 uM, or 100.6 uM. In another embodiment, the initial concentration of a chemical is about 1 mM, 3 mM, 6 mM, 9 mM, 11.5 mM, 14.2 mM, 16.9 mM, 19.6 mM, 22.3 mM, 25 mM, 27.7 mM, 30.4 mM, 33.1 mM, 35.8 mM, 38.5 mM, 41.2 mM, 43.9 mM, 46.6 mM, 49.3 mM, 52 mM, 54.7 mM, 57.4 mM, 60.1 mM, 62.8 mM, 65.5 mM, 68.2 mM, 70.9 mM, 73.6 mM, 76.3 mM, 79 mM, 81.7 mM, 84.4 mM, 87.1 mM, 89.8 mM, 92.5 mM, 95.2 mM, 97.9 mM, or 100.6 mM. In another embodiment, concentration of a chemical introduced to the culture can be increased by about 1.1 fold, 1.3 fold, 1.5 fold, 1.7 fold, 2.0 fold, 2.2 fold, 2.4 fold, 2.6 fold, 2.8 fold, 3.1 fold, 3.3 fold, 3.5 fold, 3.7 fold, 3.9 fold, 4.2 fold, 4.4 fold, 4.6 fold, 4.8 fold, 5.0 fold, 5.3 fold, 5.5 fold, 5.7 fold, 5.9 fold, 6.1 fold, 6.4 fold, 6.6 fold, 6.8 fold, 7.0 fold, 7.2 fold, 7.5 fold, 7.7 fold, 7.9 fold, 8.1 fold, 8.3 fold, 8.6 fold, 8.8 fold, 9.0 fold, 9.2 fold, 9.4 fold, 9.7 fold, 9.9 fold, or 10.1 fold. In another embodiment, concentration of a chemical introduced to the culture can be increased by about 10 fold, 20 fold, 50 fold, 70 fold, 100 fold, 119 fold, 142 fold, 165 fold, 188 fold, 211 fold, 234 fold, 257 fold, 280 fold, 303 fold, 326 fold, 349 fold, 372 fold, 395 fold, 418 fold, 441 fold, 464 fold, 487 fold, 510 fold, 533 fold, 556 fold, 579 fold, 602 fold, 625 fold, 648 fold, 671 fold, 694 fold, 717 fold, 740 fold, 763 fold, 786 fold, 809 fold, 832 fold, 855 fold, 878 fold, 901 fold, 924 fold, 947 fold, 970 fold, 993 fold, or 1016 fold.

In one embodiment, a pre-determined amount of chemical is introduced to the continuous culture devices described herein by injecting the chemical into the culture chamber. In another embodiment, the chemical is dissolved into a liquid and introduced to the devices as part of the culture medium. In another embodiment, the liquid is water. In another embodiment, the liquid is a buffered solution such as phosphate buffer, Tris buffer, Carbonate buffer. A buffer is selected depending on the circumstances and types of the microorganism, considering the effect of buffering chemicals and salts on the growth of the microorganism. In another embodiment, the chemical is added to the culture media as a slowly-dissolving pellet. In another embodiment, a pellet is a tablet. In another embodiment, a pellet is a solid compacted granule. In another embodiment, a salt of the chemical is added to the culture medium. In another embodiment, the chemical is added to culture chamber via an aerosol. In another embodiment, a continuous stream of aerosol is provided to the culture chamber via an injector. In another embodiment, the chemical is aerosolized and injected once to the culture chamber. In another embodiment, the aerosolized chemical is injected regularly over a period of time. In another embodiment, gas-permeable tubing is used as a culture chamber and the section of tubing where the culture is contained is sealed in a gas chamber. In another embodiment, the culture device is placed in a gas-tight chamber. In another embodiment, the culture device is placed in a gas-tight room.

In one embodiment, a microorganism is evolved to tolerate one or more herbicide or insecticide described herein. Chemical herbicides include, but are not limited to, lipid biosynthesis inhibitors such as chlorazifop, clodinafop, clofop, cyhalofop, diclofop, fenoxaprop, fenoxaprop-p, fenthiaprop, fluazifop, fluazifop-P, haloxyfop, haloxyfop-P, isoxapyrifop, metamifop, propaquizafop, quizalofop, quizalofop-P, trifop, alloxydim, butroxydim, clethodim, cloproxydim, cycloxydim, profoxydim, sethoxydim, tepraloxydim, tralkoxydim, butylate, cycloate, diallate, dimepiperate, EPTC, esprocarb, ethiolate, isopolinate, methiobencarb, molinate, orbencarb, pebulate, prosulfocarb, sulfallate, thiobencarb, tiocarbazil, triallate, vernolate, benfuresate, ethofumesate and bensulide; ALS inhibitors such as amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac; photosynthesis inhibitors such as atraton, atrazine, ametryne, aziprotryne, cyanazine, cyanatryn, chlorazine, cyprazine, desmetryne, dimethametryne, dipropetryn, eglinazine, ipazine, mesoprazine, methometon, methoprotryne, procyazine, proglinazine, prometon, prometryne, propazine, sebuthylazine, secbumeton, simazine, simeton, simetryne, terbumeton, terbuthylazine, terbutryne, trietazine, ametridione, amibuzin, hexazinone, isomethiozin, metamitron, metribuzin, bromacil, isocil, lenacil, terbacil, brompyrazon, chloridazon, dimidazon, desmedipham, phenisopham, phenmedipham, phenmedipham-ethyl, benzthiazuron, buthiuron, ethidimuron, isouron, methabenzthiazuron, monoisouron, tebuthiuron, thiazafluoron, anisuron, buturon, chlorbromuron, chloreturon, chlorotoluron, chloroxuron, difenoxuron, dimefuron, diuron, fenuron, fluometuron, fluothiuron, isoproturon, linuron, methiuron, metobenzuron, metobromuron, metoxuron, monolinuron, monuron, neburon, parafluoron, phenobenzuron, siduron, tetrafluoron, thidiazuron, cyperquat, diethamquat, difenzoquat, diquat, morfamquat, paraquat, bromobonil, bromoxynil, chloroxynil, iodobonil, ioxynil, amicarbazone, bromofenoxim, flumezin, methazole, bentazone, propanil, pentanochlor, pyridate, and pyridafol; protoporphyrinogen-IX oxidase inhibitors such as acifluorfen, bifenox, chlomethoxyfen, chlornitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidon-ethyl, flumiclorac, flumioxazin, flumipropyn, fluthiacet, thidiazimin, oxadiazon, oxadiargyl, azafenidin, carfentrazone, sulfentrazone, pentoxazone, benzfendizone, butafenacil, pyraclonil, profluazol, flufenpyr, flupropacil, nipyraclofen and etnipromid; bleacher herbicide such as metflurazon, norflurazon, flufenican, diflufenican, picolinafen, beflubutamid, fluridone, fluorochloridone, flurtamone, mesotrione, sulcotrione, isoxachlortole, isoxaflutole, benzofenap, pyrazolynate, pyrazoxyfen, benzobicyclon, amitrole, clomazone, aclonifen, 4-(3-trifluoromethylphenoxy)-2-(4-trifluoromethylphenyl)pyrimidine, and 3-heterocyclyl-substituted benzoyl derivatives; EPSP synthase inhibitors such as glyphosate; glutamine synthase inhibitors such as glufosinate and bilanaphos; DHP synthase inhibitors such as asulam; mitose inhibitors such as benfluralin, butralin, dinitramine, ethalfluralin, fluchloralin, isopropalin, methalpropalin, nitralin, oryzalin, pendimethalin, prodiamine, profluralin, trifluralin, amiprofos-methyl, butamifos, dithiopyr, thiazopyr, propyzamide, tebutam, chlorthal, carbetamide, chlorbufam, chlorpropham and propham; VLCFA inhibitors such as acetochlor, alachlor, butachlor, butenachlor, delachlor, diethatyl, dimethachlor, dimethenamid, dimethenamid-P, metazachlor, metolachlor, S-metolachlor, pretilachlor, propachlor, propisochlor, prynachlor, terbuchlor, thenylchlor, xylachlor, allidochlor, CDEA, epronaz, diphenamid, napropamide, naproanilide, pethoxamid, flufenacet, mefenacet, fentrazamide, anilofos, piperophos, cafenstrole, indanofan and tridiphane; cellulose biosynthesis inhibitors such as dichlobenil, chlorthiamid, isoxaben and flupoxam; decoupler herbicide such as dinofenate, dinoprop, dinosam, dinoseb, dinoterb, DNOC, etinofen and medinoterb; auxin herbicide such as clomeprop, 2,4-D, 2,4,5-T, MCPA, MCPA thioethyl, dichlorprop, dichlorprop-P, mecoprop, mecoprop-P, 2,4-DB, MCPB, chloramben, dicamba, 2,3,6-TBA, tricamba, quinclorac, quinmerac, clopyralid, fluoroxypyr, picloram, triclopyr and benazolin; auxin transport inhibitors such as naptalam, diflufenzopyr; benzoylprop, flamprop, flamprop-M, bromobutide, chlorflurenol, cinmethylin, methyldymron, etobenzanid, fosamine, metam, pyributicarb, oxaziclomefone, dazomet, triaziflam and methyl bromide.

Chemical insecticides include, but are not limited to, organophosphates such as acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon; Carbamates such as alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate; Pyrethroids such as allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, gamma-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin; chitin synthesis inhibitors such as benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; ecdysone antagonists such as halofenozide, methoxyfenozide, tebufenozide, azadirachtin; juvenoids such as pyriproxyfen, methoprene, fenoxycarb; lipid biosynthesis inhibitors such as spirodiclofen, spiromesifen, spirotetramat; nicotinic receptor agonists/antagonists compounds such as clothianidin, dinotefuran, imidacloprid, thiamethoxam, nitenpyram, acetamiprid, thiacloprid; thiazol compounds; GABA antagonist compounds such as acetoprole, endosulfan, ethiprole, fipronil, vaniliprole, pyrafluprole, pyriprole, and phenylpyrazole compounds; macrocyclic lactone insecticide such as abamectin, emamectin, milbemectin, lepimectin, and spinosad; METI I compounds such as fenazaquin, pyridaben, tebufenpyrad, tolfenpyrad, flufenerim; METI II and III compounds such as acequinocyl, fluacyprim, hydramethylnon; uncoupler compounds such as chlorfenapyr; oxidative phosphorylation inhibitor compounds such as cyhexatin, diafenthiuron, fenbutatin oxide, propargite; moulting disruptor compounds such as cyromazine; mixed function oxidase inhibitor compounds such as piperonyl butoxide; sodium channel blocker compounds such as indoxacarb, metaflumizone; and others such as benclothiaz, bifenazate, cartap, flonicamid, pyridalyl, pymetrozine, sulfur, thiocyclam, flubendiamide, cyenopyrafen, flupyrazofos, cyflumetofen, and amidoflumet.

In addition to herbicide and insecticide, methods, devices, and compositions described herein are applicable to evolving a microorganism to acquire resistance against a fungicide. Examples of a fungicide include, but are not limited to, strobilurins such as azoxystrobin, dimoxystrobin, enestroburin, fluoxastrobin, kresoxim-methyl, metominostrobin, picoxystrobin, pyraclostrobin, trifloxystrobin, orysastrobin, methyl(2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate, methyl 2-(ortho#2,5-dimethylphenyloxymethylene)phenyl)-3-methoxyacrylat-e; carboxamides such ascarboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-c-arboxamide, N-(4′-trifluoromethylbiphenyl-2-yl)-4-difluoromethyl-2-methylthiazole-5-c-arboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazol-e-4-carboxamide, N-(2-cyanophenyl)-3,4-dichloroisothiazole-5-carboxamide; carboxylic acid morpholides: dimethomorph, flumorph; benzamides: flumetover, fluopicolide (picobenzamid), zoxamide; other carboxamides: carpropamid, diclocymet, mandipropamid, N-(2-(443-(4-chlorophenyl)prop-2-ynyloxy]-3-methoxyphenypethyl)-2-methanesulfonylamino-3-methylbut-yramide, N-(2-(4-[3-(4-chlorophenyl)prop-2-ynyloxy]-3-methoxyphenypethyl)-2-ethanesulfonylamino-3-methylbutyramide; azoles such astriazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole; imidazoles: cyazofamid, imazalil, pefurazoate, prochloraz, triflumizole; benzimidazoles: benomyl, carbendazim, fuberidazole, thiabendazole; other azoles: ethaboxam, etridiazole, hymexazole; nitrogenous heterocyclyl compounds such aspyridines: fluazinam, pyrifenox, 3-[5-(4-chlorophenyl)-2,3-dimethylisoxazolidin-3-yl]-pyridine; pyrimidines: bupirimate, cyprodinil, ferimzone, fenarimol, mepanipyrim, nuarimol, pyrimethanil; piperazines: triforine; pyrroles: fludioxonil, fenpiclonil; morpholines: aldimorph, dodemorph, fenpropimorph, tridemorph; dicarboximides: iprodione, procymidone, vinclozolin; others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1 -yl)-6-(2,4,6-trifluorophenyl)-[1,2,4]tria-zolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazo-le-1-sulfonamide; carbamates and dithiocarbamates such asdithiocarbamates: ferbam, mancozeb, maneb, metiram, metam, propineb, thiram, zineb, ziram; carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl) carbamate; other fungicides such asguanidines: dodine, iminoctadine, guazatine; antibiotics: kasugamycin, polyoxins, streptomycin, validamycin A; organometallic compounds: fentin salts; sulfur-containing heterocyclyl compounds: isoprothiolane, dithianon; organophosphorus compounds: edifenphos, fosetyl, fosetyl-aluminum, iprobenfos, pyrazophos, tolclofos-methyl, phosphorous acid and its salts; organochlorine compounds: thiophanate-methyl, chlorothalonil, dichlofluanid, tolylfluanid, flusulfamide, phthalide, hexachlorbenzene, pencycuron, quintozene; nitrophenyl derivatives: binapacryl, dinocap, dinobuton; inorganic active compounds: Bordeaux mixture, copper acetate, copper hydroxide, copper oxychloride, basic copper sulfate, sulfur; others: spiroxamine, cyflufenamid, cymoxanil, or metrafenone.

Containment Mechanisms

In one embodiment, an EMO comprises a self-destruct mechanism. In another embodiment, the microorganism is a bacterium, virus, algae, fungus, or a microorganism capable of sporulation. In another embodiment, the microorganism is a bacterium. In another embodiment, the bacterium is an E. coli strain. In another embodiment, a genetic engineering technique known in the art is used to introduce a self-destruct mechanism into a microorganism. In another embodiment, the mechanism is a suicidal vector, (e.g., a vector comprising multiple transposons), inserted into a genetically modified microorganism to ensure self-destruction after the number of cell division reaches certain threshold. Another example of genetic modification is metabolic block where the microorganism dies in the absence of a particular food source.

Methods, devices, and compositions disclosed herein are useful to evolve strains to acquire self-destructive mechanisms without resorting to genetic engineering. To evolve for self-destruction, a microorganism is exposed to various environmental stresses. A strain sensitive to a particular stress is selected. In one embodiment, a strain sensitive to temperature drop or increase is selected by continuously culturing the microorganism in one temperature and then shifting the temperature to selection temperature. Selection is made based on the growth rate or number of cells surviving at the selection temperature. A strain sensitive to temperature drop is useful, for example, for spraying in a field in late summer where a temperature drop is expected to occur within weeks. A useful temperature difference (either drop or increase) for self-destruction can be as little as 1 degree Celsius to as large as 12 degree Celsius. Other types of environmental stresses include, but are not limited to, humidity, heat, and UV. In another embodiment, a microorganism is evolved to acquire temperature sensitivity at 28° C. The microorganism is first evolved to growth at 37° C. The evolved strain is then exposed to abrupt temperature shift to 28° C. The growth rate at 28° C. is then monitored for a period. Of the strains growing at 28° C., the most slow-growing strain is selected and the process is repeated. At the end of every round of the process the growth rate of a microorganism is compared to a microorganism selected from previous round. By repeating the process, a strain for which a microorganism dies or shows a precipitous drop in growth rate upon temperature shift is selected.

In one embodiment, a genetically engineered microorganism is evolutionary modified to acquire one or more useful traits. In another embodiment, a genetically engineered microorganism is a microorganism containing a suicide mechanism. In another embodiment, the suicide mechanism is an inducible cassette expressing a toxin. In another embodiment, the toxin is Colicin. In another embodiment, the toxin is ricin. In another embodiment, the toxin is sarcotoxin I. Non-limiting examples of antimicrobial protein include magainins, alamethicin, pexiganan, polyphemusin, LL-37, defensins and protegrins. In another embodiment, a gene encoding one or more toxins is operably coupled to an inducible promoter for an inducible expression of the toxin in the microorganism. An inducible promoter can be any metabolically inducible promoter, such as arabinose operon, chemically inducible promoter such as tetracycline, or temperature inducible promoter, such as heat shock protein promoter. In another embodiment, an artificially evolved microorganism does not comprise a self-destruct mechanism.

Sporulation and Spores

In one embodiment, a microorganism is artificially evolutionarily modified to acquire modified sporulation or modified spores. In another embodiment, the modification is an increased amount of sporulation. In another embodiment, the microorganism is a bacterium, virus, algae, fungus, or other microorganism capable of sporulation. In another embodiment, the microorganism is a bacterium. In another embodiment, the bacterium is an E. coli strain. In another embodiment, the microorganism is a fungus. In another embodiment, the fungus is M. anisopliae. In another embodiment, the fungus is M. flavoviridae.

In one embodiment, a microorganism is placed in continuous culture for a period of time and then removed from the culture. The removed culture is dried. Dried spores are then placed back in a continuous culture. In another embodiment, the cycle of culturing, drying and re-culturing using a continuous culture device described herein is repeated to provide artificial selection pressure on the culture, resulting in adaptation to the cyclical changes in environmental conditions, which leads to increased or better sporulation or more efficient spores.

In one embodiment, increased sporulation increases the quantity of spores produced. The abundance of spores produced from an artificially evolved microorganism can be 1.1, 1.2, 1.5, 1.75, 2.0, 2.5, 3, 4, 5, 6, 7, 8, 9, 10, 20, 30, 50, 70, 100, 200, 300, 500, 750, 1,000, 2,000, 3,000, 5,000, 7,000, or 10,000 times more than the number of spores produced by a wild-type microorganism.

In one embodiment, methods, device, and compositions described herein modify the characteristics of sporulation in ways other than affecting the quantity of spores, resulting in modified spores. In another embodiment, a modified spore can be any spore evolved to acquire enhanced efficiency as a biocontrol agent. Examples of enhanced efficiency include, but are not limited to, increased virulence, increased viability, increased dispersability, and combinations thereof.

In one embodiment, modified spores are placed in a continuous culture device described herein to further acquire increased sporulation. In another embodiment, a microorganism is artificially evolved so that it produces spores modified to have increased viability. In another embodiment, a modified spore is viable for about 1 day to 10 years after it is produced, such as about 1-7 days, 1-4 weeks, 1-3 months, 1-6 months, 1 month-1 year, lyear, 1 day-2 years, 1 day-3 years, 1 day-4 years, 1 day-5 years, 1 day-6 years, 1 day-7 years, 1 day-8 years, 1 day-9 years, or 1 day-10 years. In another embodiment, a modified spore remains viable after exposure to very dry environmental conditions. In another embodiment, the exposure is for about 1-7 days, 1-4 weeks, 1-3 months, 1-6 months, 1 month-1 year, lyear, 2 years, 3 years, 4 years, 5 years, 6 years, 7 years, 8 years, 9 years, or 10 years. In another embodiment, a modified spore remains viable after exposure to periods of low temperature. In another embodiment, the temperature is below freezing. In another embodiment, the exposure is for about 1-7 days, 1-4 weeks, 1-3 months, 1-6 months, 1 month-1 year, lyear, 1 day-2 years, 1 day-3 years, 1 day-4 years, 1 day-5 years, 1 day-6 years, 1 day-7 years, 1 day-8 years, 1 day-9 years, or 1 day-10 years. In another embodiment, a modified spore remains viable after exposure to periods of high temperature. In another embodiment, the temperature is above about 100° F. In another embodiment, the exposure is for about 1-7 days, 1-4 weeks, 1-3 months, 1-6 months, 1 month-1 year, 1 year, 1 day-2 years, 1 day-3 years, 1 day-4 years, 1 day-5 years, 1 day-6 years, 1 day-7 years, 1 day-8 years, 1 day-9 years, or 1 day-10 years.

In one embodiment, a bacterial strain is cultured in a medium favoring increased sporulation. Examples of media compositions include, but are not limited to, adding vitamins, and reducing folic acid, inositols, thiamine, p-aminobenzoic acid, pyridoxine, or riboflavin.

In one embodiment, evolved strains are catalogued according to the degree of sporulation. For strains that do not exhibit increased sporulation, these strains are screened for sporulation defects. For strains where sporulation defects are severe enough not to produce any viable spores, these strains are utilized in conditions where containment can be difficult. In another embodiment, a strain evolved to acquire de novo sporulation characteristics is further evolved to acquire other useful traits described herein.

Thermotolerance or Cryotolerance

In one embodiment, a microorganism is artificially evolutionarily modified to acquire tolerance to temperatures colder or warmer than the temperature the unmodified microorganism normally grows at. The economic viability of microorganism-based applications, such as the production of biofuels or protecting valuable crops, is limited by microorganism's physiological growth temperature. The boundaries of growth temperature often define seasonal and geographical limits of the application. Understanding how microorganisms adapt to alternative thermal niches is useful for converting a mesophile to a thermophile or a psychrophile and vice versa. A mesophile refers to an organism with a physiological growth temperature at a range of about 15-37° C. A psychrophile refers to an organism with a physiological growth temperature at a range of about 15° C. or below. A thermophile refers to an organism with a physiological growth temperature at a range of about 37° C. or above. Thermotolerance is an adaptive behavior that a microorganism tolerates temperature higher than its physiological growth temperature and grows in that higher temperature. Cryotolerance is an adaptive behavior that a microorganism tolerates temperature lower than its physiological growth temperature and grows in that lower temperature.

In one embodiment, methods, devices, and compositions described herein are useful to artificially evolutionarily modify a microorganism to become tolerant against a range of temperatures unfavorable for the growth or survival of wild type organism. In another embodiment, the organism is a microorganism. In another embodiment, the microorganism is a bacterium, virus, algae, fungus, or a microorganism capable of sporulation. In another embodiment, the bacterium is a strain of E. coli. In another embodiment, an organism is evolved to become a mesophile. In another embodiment, an organism is evolved to become a thermophile. In another embodiment, an organism is evolved to become a psychrophile. In another embodiment, an organism acquires thermotolerance. In another embodiment, an organism acquires cryotolerance.

The processes described herein can be used to artificially evolutionarily modify a wide range of mesophiles. In one embodiment, a mesophile is evolved to a thermophile. In another embodiment, a mesophile is evolved to a psychrophile. In another embodiment, a thermophile is evolved to a mesophile. In another embodiment, a psychrophile is evolved to a mesophile. In another embodiment, a thermophile is evolved to a psychrophile. In another embodiment, a psychrophile is evolved to a thermophile. In another embodiment, a mesophile is artificially evolutionarily modified to a mesophile of unnatural temperature range. In one aspect, unnatural range can overlap with natural temperature range by as little as about 0.01° C. In another aspect, unnatural, adapted range does not overlap with natural temperature range. In another embodiment, a thermophile is artificially evolutionarily modified to a thermophile of unnatural temperature range. In another embodiment, a psychrophile is artificially evolutionarily modified to a psychrophile of unnatural temperature range. In another embodiment, a microorganism is artificially evolutionarily modified to survive at target temperature. A target temperature includes, but is not limited to, about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 34.5° C., 35° C., 35.5° C., 36° C., 36.5° C., 37° C., 37.5° C., 38° C., 38.5° C., 39° C., 39.5° C., 40° C., 40.5° C., 41° C., 41.5° C., 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 49.7° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., or 69° C.

In another embodiment, a temperature-adapted microorganism (i.e., organism adapted to grown in unnatural range of temperature) is further artificially evolutionarily modified to acquire other useful traits described herein. These useful traits include, but are not limited to, ultraviolet (UV) light tolerance, enhanced growth rate, host specificity, chemical tolerance to a herbicide, insecticide or a fungicide, an increased rate of target digestion, or characteristics useful for containment.

In one embodiment, the mesophile is a bacterial species. In another embodiment, the bacterium is an E. coli strain. In another embodiment, the E. coli K-12 MG1655 strain is evolved to a thermophile as described in the examples herein. In another embodiment, the mesophile is a fungus. In another embodiment, the fungus is a strain of Metarhizium. In another embodiment, M. anisopliae species is evolved to a thermophile as described in the examples herein.

In one embodiment, a microorganism is artificially evolutionarily modified to become thermotolerant to a temperature above those to which a wild-type microorganism is typically exposed. In another embodiment, a microorganism is evolved to become cryotolerant to a temperature below those to which a wild-type microorganism is typically exposed. To evolve a selected microorganism, the microorganism can be placed under continuous culture in which the culturing temperature is gradually adjusted to a target temperature that the evolved microorganism is adapted to grow and survive. The gradual change of temperature can be less than 0.1° C. towards the target temperature to more than 5° C. In another embodiment, the target temperature can be about 5, 4, 3, 2, 1 or 0.9, 0.8, 0.7, 0.6, 0.5, 0.4, 0.3, 0.2, or 0.1 degree above or below the natural range (i.e., the range of temperature a wild type microorganism is known to grow and survive). In another embodiment, the target temperature is about 6, 7, 8, 9, 10, 11, 12, 13, 14, 15, 16, 17, 18, 19, 20, 21, 22, 23, 24, 25, 26, 27, 28, 29, or 30° C. above or below the natural range. In another embodiment, a continuous culturing system described herein is used to evolutionarily adapt a bacterial stain. In another embodiment, a bacterial stain is artificially evolutionarily modified to grow at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 34.5° C., 35° C., 35.5° C., 36° C., 36.5° C., 37° C., 37.5° C., 38° C., 38.5° C., 39° C., 39.5° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., or 69° C. In another embodiment, a fungal stain is artificially evolutionarily modified to grow at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 34.5° C., 35° C., 35.5° C., 36° C., 36.5° C., 37° C., 37.5° C., 38° C., 38.5° C., 39° C., 39.5° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., or 69° C.

In one embodiment, a microorganism is artificially evolutionarily modified to acquire an ability to grow and survive at a temperature lower than that of the natural microorganism. Adapting to a colder environment than the microorganism's natural habitat is useful as it would expand the applicable area of the evolved microorganism. In another embodiment, a microorganism is evolved to acquire robust growth and survival at cold temperature. In another embodiment, a microorganism evolved to adapt to cold temperature is a biocontrol agent. In another embodiment, a microorganism evolved to adapt to cold temperature is a biocontrol agent against a species classified in the nematode Phylum. In another embodiment, a target cold temperature is about 5, 4, 3, 2, 1 0.9, 0.8, 0.7, 0.6, 0.5, 0.4, 0.3, 0.2, or 0.1° C. below the natural temperature range of a wild type microorganism. The natural temperature range of wild type microorganism as used herein refers to the normal temperature range that the wild type microorganism is known to grow and survive. In another embodiment, the target temperature is about 6, 7, 8, 9, 10, 11, 12, 13, 14, 15, 16, 17, 18, 19, 20, 21, 22, 23, 24, 25, 26, 27, 28, 29, or 30° C. below the natural temperature range of a wild type microorganism. In another embodiment, a microorganism growing at 25° C. is evolved to grow at 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., 0.5° C., 0.3° C., or 0.1° C. In another embodiment, a continuous culturing system described herein is used to evolutionarily adapt a microorganism to grow at a temperature range below its natural temperature range. In another embodiment, the microorganism is a bacterium. In another embodiment, the microorganism is a fungus. In another embodiment, the microorganism is yeast.

In one embodiment, a microorganism is artificially evolutionarily modified tolerate to an oscillating temperature. In another embodiment, a microorganism is evolved to a temperature oscillating between about 8° C. to about 37° C. within 24-hour period. In another embodiment, a microorganism is evolved to a temperature oscillating between about 8° C. to about 37° C. within 12-hour period. In another embodiment, a microorganism is evolved to a daytime temperature ranging between about 12° C. to 42° C. and a nighttime temperature ranging between about −5° C. to about 18° C. In another embodiment, a microorganism is adopted to withstand temperature differences within 24-hour period of about 5, 6, 7, 8, 9, 10, 11, 12, 13, 14, 15, 16, 17, 18, 19, 20, 21, 22, 23, 24, 25, 26, 27, 29, or 30° C. In another embodiment, a microorganism evolved to withstand a vastly oscillating temperature range is further evolved to grow under UV exposure. In another embodiment, methods described herein are used to further acquire UV resistance trait. In another embodiment, a UV-tolerance, temperature tolerant strain is further evolved to grow on unnatural insect host. In another embodiment, target insects include, but are not limited to cockroaches, termites, mosquitoes and grasshoppers. In another embodiment, evolved strains are sampled from continuous cultures, allowed to sporulate and passaged through the target insect to maintain sporulation capability and pathogenicity. In another embodiment, the microorganism is a bacterium. In another embodiment, the microorganism is a fungus. In another embodiment, the microorganism is Beauveria bassiana. In another embodiment, the microorganism is Metarhizium anisopliae.

In one embodiment, a microorganism is artificially evolutionarily modified to acquire tolerance to temperatures above that in which it normally grows. In another embodiment, the microorganism is a mesophile. In another embodiment, the mesophile is a bacterium. In another embodiment, the bacterium is E. coli K-12 MG1655. In another embodiment, a thermophile is a mesophile adapted to robust grow at about 48.5° C. In another embodiment, a mesophile adapted to grow at about 48.5° C. is a strain originated from E. coli K-12 MG1655. In another embodiment, a thermophile is a mesophile capable of colonizing thermal environments exceeding about 45° C. An example of thermal environment includes soil, sea, or air having the temperature of about 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., or 69° C.

In one embodiment, a mesophile is artificially evolutionarily modified to a thermophile capable of thriving in a range of temperatures unfavorable for the growth or survival of the original mesophile. In another embodiment, the mesophile is a bacterium. In another embodiment, a mesophile is evolved to become a thermophile living at a temperature above those to which a mesophile is typically exposed. In another embodiment, a mesophile is evolved to become thermotolerant to a temperature above those to which a mesophile is typically exposed. A candidate mesophile can be selected based on having a useful trait such as insecticidal trait. In another embodiment, a selected mesophile is evolved to become a thermophile or a psychrophile. To evolve a selected mesophile, the mesophile is placed under continuous culture in which the culturing temperature is gradually adjusted to a target temperature that the evolved microorganism adapts to grow and survive.

In one embodiment, acquisition of thermophily by a mesophile is confirmed as described herein. In another embodiment, evolved strains are taken out of cryopreservative condition by re-streaking on a culture medium at 37° C. The growth or evolved thermophile at adapted temperature is tested in a typical laboratory culture condition to ensure that the adaptation that has occurred is independent of the growth conditions utilized in obtaining thermophily.. In another embodiment, the growth of an evolved thermophile is tested at between about 40-70° C. by culture on a solid or in a liquid media. In another embodiment, an evolved thermophile can grow at about 40, 41, 42, 43, 44, 45, 46. 47. 48. 49. 50, 51, 52, 53, 54, 55, 56, 57, 58, 59, 60, 61, 62, 63, 64, 65, 66, 67, 68, 69, or 70° C.

In one embodiment, an evolved thermophile is EVG1031, EVG1041, EVG1058 or EVG1064. In another embodiment, the evolved thermophile is EVG1064 (FIG. 5A, B). The EVG1064 strain grows at 48.5° C. on solid media or at 48.0° C. in batch liquid culture (FIG. 5B). In another embodiment, the growth of an evolved E. coli strain is compared to an un-evolved E. coli MG1655, which can be streaked on solid media or grown in liquid media, such as at 48.5° C. or 48.0° C., respectively.

As described herein, to further characterize the evolved organism, doubling time of a culture can be measured. In onr embodiment, doubling time of an evolved thermophile is measured between its evolved temperature and its un-evolved, mesophilic growth temperature. In another embodiment, doubling time of EVG1064 is measured between its evolved temperature and its un-evolved temperature. In another embodiment, the evolved temperature for EVG1064 is 48° C. and its un-evolved growth temperature is 37° C. In another embodiment, EVG1064's doubling time at 37° C. is 0.74 per hour while its doubling time at 48° C. is 0.65 per hour.

Doubling time can be expressed in terms of the culture's optimal growth temperature (Tₒₚₜ) or Tₘₐₓ. Tₒₚₜ refers to temperature at which maximum growth occurs. Tₘₐₓ refers to maximum temperature at which the rate of growth is zero. In one embodiment, doubling time for EVG1064 is increased at 37° C. (0.74 per hour) when compared to 48° C. (0.65 per hour). The length of lag phase of an evolved thermophile can also be measured and compared between its thermophilic temperature and its mesophilic temperature. In another embodiment, the lag phase of EVG1064 growing at 48° C. is longer than at 37° C. In another embodiment, EVG1064's lag phase at 37° C. is about 1 hour. In another embodiment, EVG1064's lag phase at 48° C. is about 8 hours (FIG. 5C).

To further characterize the adaptation mechanisms of an evolved thermophile, the genome of an evolved thermophile is sequenced. The genomic sequence and optionally the order of occurrence of one or more mutations in an artificially evolved organism is determined and compared to an original wild type organism. In one embodiment, whole genome sequencing is used to determine the genotype of an organism.

Field Application

In one embodiment, an EMO is used as a better biocontrol agent. In another embodiment, an EMO is used as a better biocontrol agent without a chemical pesticide. In another embodiment, an EMO is used as a better biocontrol agent with a chemical pesticide.

In one embodiment, an EMO has high target specificity. In another embodiment, a large area of mixed vegetation can be treated with an EMO, without a noticeable harmful effect to the environment. In another embodiment, an EMO does not leave environmentally harmful chemical residues. In another embodiment, a production of an EMO is cheaper and safer than that of a chemical pesticide. In another embodiment, extended use of a biocontrol agent to inhibit or kill a target pest induces less resistance in the target pest than use of a chemical pesticide for the same length of time.

In one embodiment, an EMO is a bacterium. In another embodiment, an EMO is a fungus. In another embodiment, an EMO is yeast. In another embodiment, a strain of Bacillus subtilis is used to control plant pathogens. In another embodiment, strains of Trichoderma spp. and Ampelomyces quisqualis are used to control grape powdery mildew. In another embodiment, a strain of Bacillus thuringiensis is used to cause lethal disease in the Order of Lepidoptera, Coleoptera or Diptera. In another embodiment, a strain of Beauveria bassiana or Metarhizium anisopliae is used as biocontrol agent.

Methods and devices described herein can be used to expand geographical and seasonal ranges of a biocontrol microorganism. For a psychrophilic biocontrol microorganism, adaptation to warmer temperature can expand its use in lower latitude areas than its natural habitat. Adaptation to warmer temperature can also extend its seasonal range in its natural geographical habitat. For a thermophilic biocontrol microorganism, adaptation to colder temperature can expand its use in higher latitude areas than its natural habitat. Adaptation to colder temperature can extend its use in colder season than its natural seasonal range. For example, B. subtilis can be evolved to robustly grow below 15° C. and thereby expanding its utility in cold soil.

In one embodiment, methods for adapting a microorganism described herein can be used to expand the range of insects targeted by said microorganism. For example, a strain of Bacillus thuringiensis can be artificially evolved by methods described herein (e.g., growing on insect debris of a closely related species) to become lethal to insects species in addition to insects of the Order of Lepidoptera, Coleoptera or Diptera. In addition, by evolving a biocontrol microorganism on insect's larvae as described herein, a known biocontrol agent can adopt a lavicidal trait.

In one embodiment, methods for adapting a microorganism described herein are useful for expanding applicability of the microorganism. By building chemical tolerance toward one or more agricultural chemicals described herein (e.g. insecticide, herbicide, fungicide), the microorganism can be used with, before, or after chemical treatment. For example, Metarhizium anisopliae can be evolved to tolerate one or more chemical insecticide described herein for its use in the field where chemical insecticide is present. To build tolerance in a microorganism, popular insecticides for cornfield such as thiamethoxam, captan, diazinon, lindane, metalaxyl, or vitavax can be gradually introduced to a continuous culture device described herein.

Depending on the prevailing circumstances such as the size of crop field and the condition of soil, the EMOs described herein are packaged as emulsifiable concentrates, suspension, concentrates, directly sprayable, dilutable solutions, spreadable pastes, dilute emulsions, wettable powders, soluble powders, dispersible powders, dusts, granules or encapsulations in polymeric substances.

In one embodiment, an EMO is granulated and deposited into the soil. In another embodiment, a biocontrol bacterium evolved by methods described herein is packaged as granules and deposited into the soil. In another embodiment, an evolved microorganism is mixed with fertilizer and deposited into the soil. In another embodiment, the biocontrol bacterium is an evolved B. thuringiensis. In another embodiment, deposition process is motorized to reach deep into the soil to protect plant from root pesticide. In another embodiment, deposition takes place at the time of planting to protect the seed.

In one embodiment, an EMO is sporulated and the spore is sprayed by spraying means. Spraying means includes land spraying device such as high flotation applicator equipped with a boom, a back-pack sprayer, nurse trucks or tanks or air spraying device such as an airplane or a helicopter. In another embodiment, a spraying device is pressured. In another embodiment, a spraying device is hand-operated to reach underside of a plant. In another embodiment, artificially evolved Metarhizium anisopliae spores are sprayed on commercially valuable crop.

In one embodiment, yeast is used to clean up chemical insecticide. In another embodiment, a strain of yeast is adapted to a particular soil condition by continuous culture methods described herein. The adapted yeast strain is applied to soil by a spraying device or being directly deposited into the soil. In another embodiment, a strain of yeast is adapted to a composition of agricultural solid waste such as mixture of leaves and chemical insecticide. In another embodiment, a culture of adapted yeast is applied to agricultural solid waste for its safe disposal.

In one embodiment, initial concentration of the an EMO is determined in a small-scale setting. In another embodiment, multiple containers are prepared in which twenty to thirty arthropods such as aphids or mites are placed in each container. Evolved microorganisms are applied in a single application at a controlled volume of 2, 4, 6, 8, and 10 ml (1×10⁶ cells/ml) directly on to arthropods with a standard calibrated spray unit. The containers are then examined under a dissection microscope and the number of live and dead arthropods is recorded at 24 hours, 48 hours, and 72 hours post treatment. The results are then evaluated as to the mortality rate of the aphid or mites.

Formulations

In one embodiment, an EMO is formulated to a product. In another embodiment, evolved spores are formulated to a product. In another embodiment, spores are collected and concentrated as a powder. In one embodiment the spores are bacterial spores. In another embodiment the spores are fungal spores. In another embodiment the spores are algal spores. In another embodiment, a filtering unit and a vacuum is used to collect and concentrate spores. In another embodiment, fungal bodies which contain spores are collected and dried as powder. In another embodiment, bacteria which contain spores are collected and dried as powder. In another embodiment, algae which contain spores are collected and dried as powder. In another embodiment, the powder is mixed with water. In another embodiment, the powder is mixed with water containing carrier. An example of carrier includes, but is not limited to, sellite, kaolin, or a sugar such as starch, sucrose or glucose. In another embodiment, a water-dissolved powder is packaged in a water-tight bag or in a container connected with a sprayer unit described herein (e.g., hand-operated sprayer equipped with a nozzle or a motorized sprayer). In another embodiment, a surfactant is added to formulation to improve the dispersability and spreadability of fungus body during spraying. An example of a surfactant includes, but is not limited to, polyoxyethylene alkyl ether and ester, polyoxyethylene alkyl phenyl ether and ester, polyoxyethylene alkyl fatty acid ester, or polyoxyethylene sorbitan fatty acid ester.

In one embodiment, evolved microbial cells are harvested and dried. In another embodiment, drying is accomplished by lyophilization. In another embodiment, drying is accomplished by freeze-drying. In another embodiment, the harvested microbial cells are resuspended in a buffered solution prior to drying. In another embodiment, the buffered solution is Tris buffer. In another embodiment, the buffered solution is a phosphate buffer. The selection of the buffer is determined by the pH in which the viability of the microorganism is maximized. In another embodiment, the harvested culture is resuspended in a buffer containing sugars such as dextrose or starch and/or oil. In another embodiment, the amount of sugars and oil is adjusted to control the viscosity of the final mixture. In another embodiment, the harvested culture is resuspended in a small volume of fresh medium mixed with oil. In another embodiment, the oil is vegetable oil.

In one embodiment, long-chain fatty acid is used instead of oil. In another embodiment, long-chain fatty acid is C10 to C30 fatty acid. As used herein, C10 to C30 refers to the number of carbon atoms per fatty acid. For example, a C10 fatty acid is a fatty acid having 10 carbon atoms. A C10 fatty acid includes, but is not limited to, a decanoic acid or its derivative. A C10 fatty acid can be saturated or containing one or more double bonds. A C30 fatty acid includes, but is not limited to, a Triacontanoic acid. A C10 to C30 fatty acid includes, but is not limited to, Decanoic acid, Undecanoic acid, Dodecanoic acid, Tridecanoic acid, Tetradecanoic acid, Pentadecanoic acid, Hexadecanoic acid, Heptadecanoic acid, Octadecanoic acid, Nonadecanoic acid, Eicosanoic acid, Heneicosanoic acid, Docosanoic acid, Tricosanoic acid, Tetracosanoic acid , Pentacosanoic acid, Hexacosanoic acid, Heptacosanoic acid, Octacosanoic acid, Nonacosanoic acid, or Triacontanoic acid. In another embodiment, the fatty acid is a stearate. In another embodiment, the fatty acid is a palmitate.

In one embodiment, dried powder or viscous mixture is placed to a formulation process to produce granules containing evolved microorganism. In another embodiment, viscous mixture is sprayed as a droplet onto a pre-warmed surface for quick drying. In another embodiment, dried powder can be used for coating such as spraying onto wetted cellulose film. The coated film can be further processed for compaction or other formulation processes described in Remington: The Science and Practice of Pharmacy (21^{st} edition, Lippincott Williams & Wilkins, 2005), which is herein incorporated by reference in its entirety.

In one embodiment, active ingredient of the formulation comprises about 0.1% to 99%, of evolved microorganism, about 1% to 99.9% of a solid or liquid adjuvant, and 0% to 25% of a surfactant. In one embodiment, the content of evolved microorganism is about 1% 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In one embodiment, the content of solid or liquid adjuvant is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.9%. In one embodiment, the content of surfactant is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In another embodiment, active ingredient is formulated as a concentrate. In another embodiment, a diluent for a concentrate is water. In another embodiment, the formulation further comprises other ingredients such as stabilizers, antifoams, viscosity regulators, binders, tackifiers as well as fertilizers.

Methods of formulating a live microorganism are further described in U.S. Pat. Application 2005/0244391, U.S. Pat. No. 7,291,328, or 6,372,209, which are herein incorporated by reference in their entirety.

### EXAMPLES

### Example 1

### A Continuous Culture Device

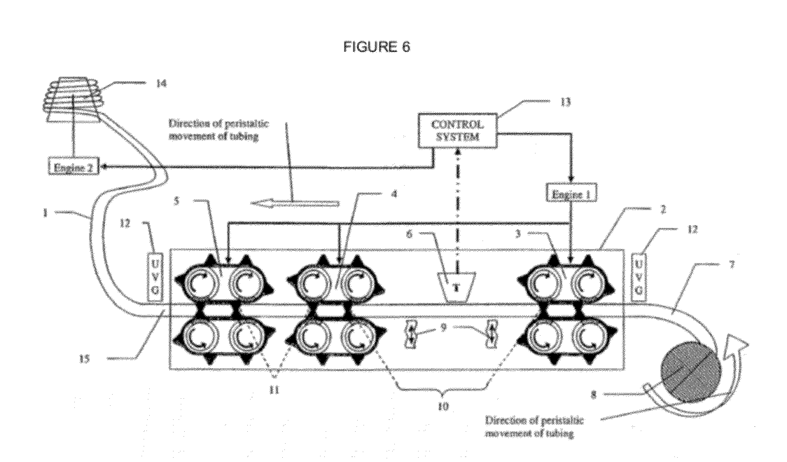

FIG. 6 displays an overall view of a possible configuration of a continuous culture device. A flexible tubing (1) contains the different regions of the device which are: upstream fresh medium region (7), growth chamber region (10), sampling chamber (11) and disposed grown culture region (15). A thermostatically controlled box (2) allows regulation of temperature according to conditions determined by user. Within the box located are the following: growth chamber (10), sampling chamber (11), upstream gate (3) defining the beginning of said growth chamber, downstream gate (4) defining the end of said growth chamber and the beginning of sampling chamber, second downstream gate (5) defining the end of the sampling chamber, turbidimeter (6) allowing the user or automated control system to monitor optical density of growing culture and to operate a feedback control system (13) as well as allowing controlled movement of the tubing on the basis of culture density (turbidostat function), and one or several agitators (9). It should be noted that the device elements listed in herein may also be located outside of, or in the absence of, a thermostatically controlled box. The fresh medium (7) is in unused flexible tubing. A barrel (8) loaded with fresh medium filled tubing is used to dispense the fresh medium and tubing during operations. An optional ultra-violet radiation gate (12) can be used. A control system (13) comprises a computer connected with means of communication to different monitoring or operating interfaces, like optical density turbidimeters, temperature measurement and regulation devices, agitators and tilting motors, etc, that allow automation and control of operations, optionally, a disposal barrel (15) can be used on which to wind up tubing containing disposed grown culture filled tubing. Disposed grown culture is located downstream of said sampling chamber. (14) represents the optional disposal barrel on which to wind up tubing containing disposed grown culture filled tubing.

### Example 2

### Evolutionary Adaptation of Filamentous Fungi

With the use of Evolugator™ technology, c strain ARSEF2575 (USDA ARS Insect Pathogenic Fungus Collection, Ithaca, N.Y.), whose normal upper thermal limit for growth is 32° C., was adapted to grow at 37° C.

Continuous Culture Setup

Briefly, directed selection occurs inside a growth chamber made of 100% silicone tubing (12.7 mm external diameter and 9.5 mm internal diameter, Saint Gobain, France) that is flexible, transparent and gas-permeable. The tubing is filled with growth medium and sterilized prior to mounting into the continuous culturing system described herein, where it is subdivided using “gates”, which are clamps that prevent the flow of medium and cultured organisms from one subdivision to the next. Between the central gates is the “growth chamber”, which has a volume of ˜10.8 mL. Oxygenation of the growth chamber is augmented beyond the permeability of the tubing by maintaining a 1.8 mL (±5%) bubble of filtered air in the growth chamber. Cultures are inoculated into the growth chamber through the tubing using sterilized syringes. The growth medium and the inner surface of the tubing are static with respect to each other, and both are regularly and simultaneously replaced by peristaltic movement of the tubing through the gates. A fresh air bubble is delivered with each dilution cycle by movement of air in predetermined volumes through the unused portion of media upstream of the growth chamber.

The gates are periodically released allowing unused medium to mix with saturated culture. The tubing is then moved and the gates reclosed essentially, the majority of the medium and growth chamber are entirely replaced during every dilution cycle. In the “new growth chamber”, culture is diluted with unused medium. The “old” growth chamber is now what is called the “sampling chamber” from which samples can be extracted by syringe without fear of contaminating the “new growth chamber”.

Dilutions were conducted automatically and controlled through specifically designed software. Dilution can be initiated at a certain cycle duration (chemostat mode), when the culture attains a certain OD (turbidostat mode) or a combination of both. Two turbidimeters (λ=680 nm, power=0.7 V) (EFS, Montagny, France) measure the optical density and are zeroed with unused growth medium prior to each experiment.

Since filamentous fungi adhere to solid surfaces, they grow along the inner surface of the “growth chamber”. Since the cells from the previous cycle adhere closest to the gate separating the “sampling chamber” and the “new growth chamber”, dividing cells will grow along the fresh chamber surface towards the gate separating the “new growth chamber” from unspent medium. Consequently, the cells that reach this gate by growing along the surface are the most recent (and presumably most fit) additions to the population, which are retained in the active culture when the tubing moves again to achieve the next dilution.

For directed evolution of M. anisopliae, the tubing was filled with Sabouraud dextrose (SAB) media and autoclaved prior to use. 2 mL of a growing culture of M. anisopliae 2575 grown in SAB was injected into the first section of the growth chamber and dilution cycles were initiated as described. Temperature was monitored using a PT100 probe (IEC/Din Class A) and regulated via a Proportional Integral & Derivative controller (West P6100™). Growth kinetics were determined using a Bioscreen C plate reader system™ (Growth Curves USA, Piscataway, N.J.) in multiple volumes of 250-300 mL. Aliquots of growing cultures were mounted on slides and examined using a PASCAL LSM5™ confocal microscope fitted with Nomarski differential interference contrast (DIC) optics.

Selection of Thermostable M. anisopliae Isolates

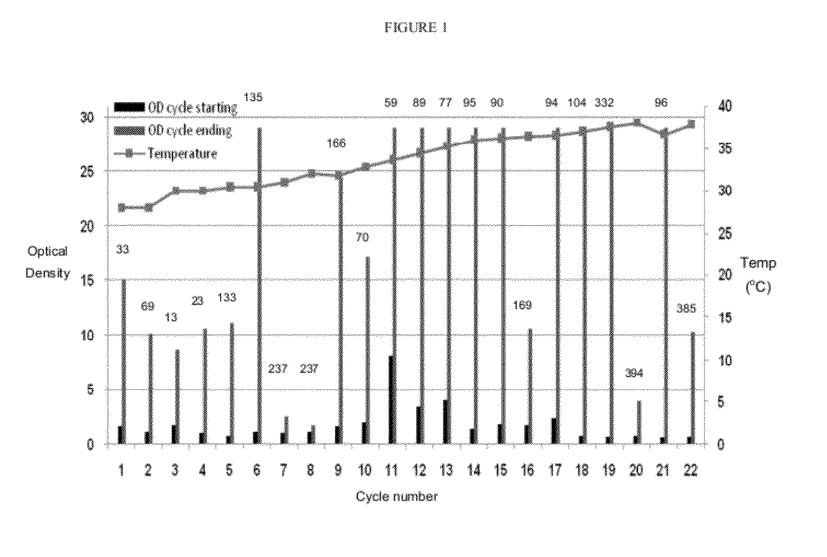

An actively growing culture of M. anisopliae was inoculated inside the growth chamber of the continuous culturing system described herein at 28° C. as described in the Methods section. Growth was monitored by optical density (OD) and dilution cycles were initiated according to OD or cycle duration. FIG. 1 presents a detailed description of 22 successive selection cycles over a 4-month period. For each cycle, the temperature of the culture chamber was recorded as well as the starting OD and ending OD. The starting OD is always low because the cells have just been diluted with fresh medium. The ending OD is higher because the cells have multiplied. FIG. 1 also shows the duration of each dilution cycle, which is the length of time the cells are allowed to grow prior to initiating a new dilution cycle.

The fungus displayed rapid growth characteristics in cycles 1-4 where the temperature increased from 28° C. to 30° C. During these cycles the culture duration was 1-2 days. Beginning at cycle 5, however, the growth rate slowed down as evidenced by an increase in the amount of time it takes to grow enough cells to initiate a dilution. This indicated that it was taking longer for favorable variants to take over the population. Moreover, the maximal cell yield (OD) dropped significantly during cycles 7 (31° C.) and 8 (32° C.), even though cells were allowed to grow for over 200 hours each time, indicating decreased overall fitness. In cycles 8 and 9, the chamber temperature was not varied significantly in order to allow variants that can grow rapidly at this temperature to take over the population. Similar phenomena, where cycle duration needed to be increased and temperature stabilized to allow fast growing variants to take over, were also seen in cycles 16 (34.6° C.) and 20 (38° C.). Two strains, termed EVG016 and EVG017 were isolated from cells cultured in cycles 18 and 22, respectively. Sequencing of the ITS1 and a fragment of the M. anisopliae specific protease Pr1 genes revealed that both isolates were derivatives of the original wild type strain.

Phenotypic Characterization of M. anisopliae Thermostable Isolates

Isolates EVG016 and EVG017 were streaked on Potato-dextrose agar (PDA) plates. Wild-type M. anisopliae (2575) typically produces green-pigmented spores (conidia) within 3-5 days of cultivation on these plates. EVG016 produced colonies that appeared less green than the wild type, whereas EVG017 produced white colonies with occasional spores visible at colony fringes or at the center of the colony. Microscopic examination revealed reduced spore production in EVG017. Conidial production in replicated solid substrate fermentation confirmed reduced sporulation. EVG016 produced a mean of 7.7×10¹¹ conidia/kg barley substrate versus 3.9×10¹² for the parent strain, a statistically significant difference (P<0.05, Student t-test). EVG017 produced less than 1% of the spores of the wild-type strain. We isolated a variant of EVG017, named EVG017g, that retained thermotolerance but was as capable of conidiation as wild type.

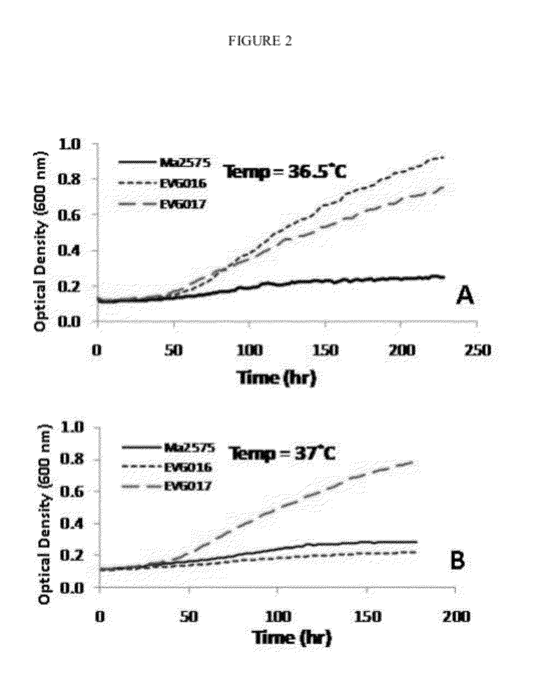

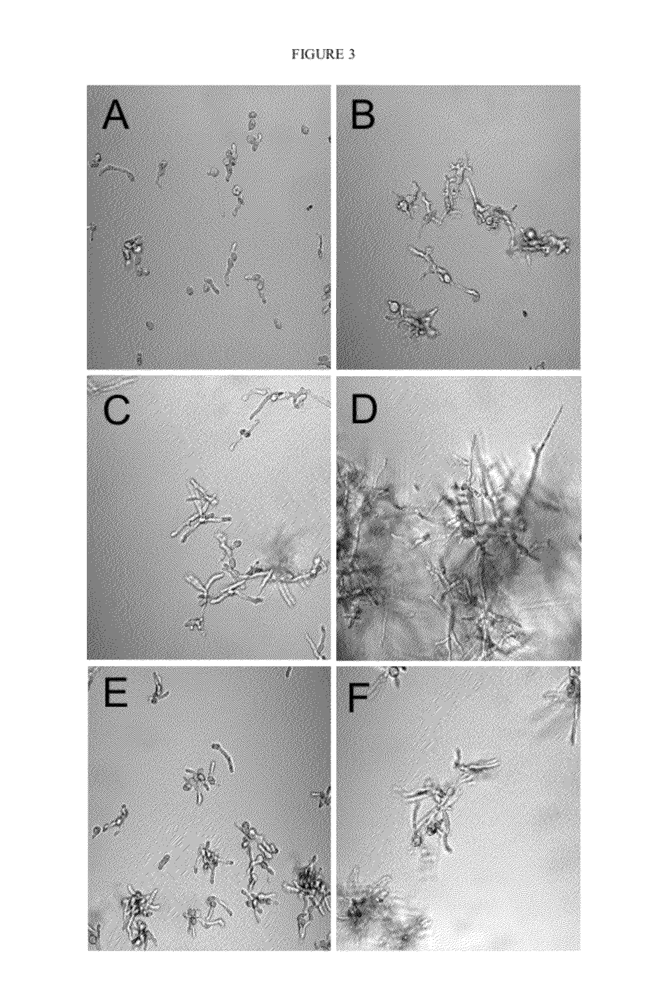

The growth characteristics of the wild-type parent, EVG016 and EVG017 in liquid media were examined at various temperatures. All three strains displayed similar growth kinetics at 28° C., whereas only EVG016 and EVG017 displayed robust growth at 35.5° C. (FIG. 2). EVG017 grew at 37° C. and no growth was evident for any of the strains at 38° C., indicating a narrow threshold for the adaptive response. Neither the wild type nor the heat adapted strains displayed appreciable radial growth at 36-37° C. when plated on solid (agar) media, although all displayed similar growth kinetics at 28° C. The strains did remain viable, and radial growth on plates was evident after a short lag period when plates were shifted from 37° C. to 28° C. Microscopic examination of the growth of the adapted and wild-type strains revealed that whereas both the wild-type and EVG016 germinated and grew across the surface of the agar, EVG017 displayed more rapid formation of appressoria than the parent and the fungal hyphae of this strain appeared to begin to penetrate the agar during the initial stages of growth. The two adapted strains also displayed different hyphal morphologies. Microscopic examination of the growing cells (in liquid culture) revealed short-tubular growth of EVG016 at 37° C., whereas EVG017 at 37° C. appeared similar to wild type grown at 28° C. (FIG. 3). Interestingly, our results indicate that the wild-type strain was able to germinate at 37° C., but failed to subsequently grow.

Sequencing of Isolated Strains

Single isolated fungal colonies (corresponding to EVG016, EVG017, and EVG017g) were re-streaked onto fresh Potato dextrose agar plates and used for identification purposes. Fungal identity was confirmed by PCR amplification and sequencing of a portion of the 5.8S rRNA with its flanking internal transcribed spacer sequences (ITS) and the M. anisopliae specific protease Pr1 as described. Primer pairs used were: (1) ITS5; 5′-gcaagtaaaagtcgtaacaagg, and ITS4; 5′-tcctccgcttattgatatgc-3′ and (2) Pr1f, 5′-gccgacttcgtttacgagcac, and Pr1r, 5′-ggaggcctcaataccagtgtc. Genomic DNA was isolated using the Qiagen DNeasy Plant mini-extraction kit according to the manufacturer's protocols (Qiagen Inc., Valencia, Calif.). PCR reactions were performed using ExTaq DNA polymerase™ (Takara Corp., Pittsburgh, Pa.). PCR products were cloned into the pCR 2.1-TOPO vector (Invitrogen, Carlsbad, Calif.) according to the manufacturer's protocols. Plasmid inserts were sequenced at the University of Florida sequencing Facility.

Gray boxes indicate values that were compared using paired student t-tests. Asterisks indicate p values*p<0.005. **p≦0.05. Error bars indicate ±1 std. deviation. cy=cyclopropane fatty acid. Δ indicates the position of the double bond or cyclopropane ring relative to the carboxyl group. 3OH=β-hydroxyl group.

Insect Bioassays

Insect bioassays against the migratory grasshopper Melanoplus sanguinipes were performed using the wild type and adapted strains. Due to the reduced sporulation of EVG017, not enough spores could be directly harvested for insect bioassays. Therefore, the strain was passaged once through M. sanguinipes by rubbing the abdomen of host insects on an agar culture of EVG017. The fungus was then re-isolated from an insect cadaver after 6 d incubation and single spores isolated. The resultant strain, EVG017g, yielded satisfactory sporulation on solid substrate at 28° C. (1.61×10¹² conidia/Kg barley), displayed the same growth kinetics and morphology as EVG017 (at 28° C. and 37° C.) and was therefore used for the insect bioassays.

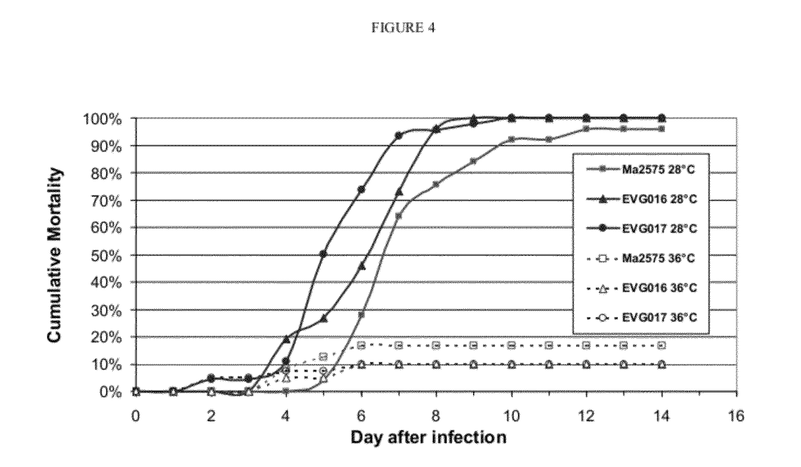

Infectivity and virulence of the wild type, EVG016 and EVG017g was evaluated using a topical 5-dose bioassay with doses bracketing the approximate LD50 based on exploratory assays. Both EVG016 and EVG017g displayed lowered infectivity as expressed by greater LD50 values compared to the wild-type parent, although due to the slopes of the dose-response curves the effect was dramatically reduced at LD95 values (FIG. 4, and Table 4).

TABLE 4
Lethal dose response data derived from topical bioassays of the parent M. anisopliae ARSEF2575,
EVG016 and EVG017g strains with adult M. sanguinipes grasshoppers at 28° C.

| strain | Assay | LD50 | 95% CL | Slope (SE) | Chi Sqr. | LD95 | 95% CL |
|---|---|---|---|---|---|---|---|
| 2575 | 1 | 799 | 63-1,722 | 1.46 (0.51) | 0.04 | 10,599 | 4,415-36,196 |
| | 2 | 1,815 | 1,174-3,042 | 1.60 (0.24) | 1.55 | 19,503 | 9.279-68,978 |
| | mean | 1,307 | | | | 15,051 | |
| | (S.D.) | (718) | | | | (6,296) | |
| EVG016 | 1 | 25,453 | 19,600-41,000 | 3.73 (0.82) | 0.75 | 70,257 | 50,534-138,856 |
| | 2 | 19,758 | 14,000-2.7400 | 2.55 (0.37) | 2.89 | 87,347 | 57,234-169,968 |
| | mean | 22,605 | | | | 78,802 | |
| | (S.D.) | (4,027) | | | | (12,084) | |
| EVG017g | 1 | 8,939 | 4,425-13,194 | 2.50 (0.64) | 0.71 | 40,787 | 25,601-127,114 |
| | 2 | 14,007 | 4,365-25,838 | 1.62 (0.41) | 2.35 | 145,180 | 71,373-765,860 |
| | mean | 11,473 | | | | 92,983 | |
| | (S.D.) | (3,584) | | | | (73,817) | |

Units for LD and confidence levels: conidia/insect.
Data are derived from two replicate bioassays using a total of 120-150 insects/bioassay.

Virulence at 28° C., in terms of Median Survival Time (ST50) calculated using Kaplan-Meier survivorship analysis, showed overall significant differences among the three fungal strains (Logrank Test Chi Square 16.45, 2 df, p =0.0003). EVG017g had a significantly faster kill (ST50), 5.5 d (95% Confidence Limits of 5.0-6.0 d), compared to 7 d (95% Confidence Limits of 7.0-7.0 d) for the wild-type parent (Logrank Test, S=−15.12; p=0.0001), for a decrease of 20%. The ST50 value for EVG016, 6.0 d (95% Confidence Limits 6.0-7.0 d), was also significantly lower than that of the wild type (Logrank Test S=−9.0632, p=0.025). EVG016 and EVG017g were not significantly different from each other (Logrank test, S=7.032; p=0.063). The LD50 and ST50 of EVG017g may have been affected by its passage through and reisolation from a grasshopper. Nevertheless, EVG017g still demonstrated reduced infectivity as did EVG016. None of the strains were pathogenic or able to cause mortality in hosts at 36° C. However, when insects infected at 36° C. were subsequently placed at 28° C., the hosts were rapidly killed by all three fungal strains, indicating that the wild type and adapted strains remained viable at 36° C., but could not cause pathogenicity and death.

Analysis of secondary non-selected traits, such as conidiation and virulence, revealed complex consequences of thermal adaptation. For example, EVG016 showed decreased infectivity when compared to wild type as measured by LD50, yet was not significantly less infective than wild type as measured by LD95. These results could simply be due to the long term culture of EVG016 in rich liquid media, condition that are known to be able to cause attenuation of pathogenicity. However, the ST50 value for EVG016 was significantly lower than that of wild type, i.e. it was a better pathogen. Absent additional thermotolerant isolates it is difficult to determine if the increased pathogenicity is associated with the thermotolerant phenotype or was a trait that was selected for serendipitously. EVG017, our second isolate from the same lineage, showed greatly impaired conidiation that could, in part, be offset or recovered by passage of the adapted isolate through an insect host. The resulting variant, EVG017g, maintained thermotolerance after passage through the insect and showed increased virulence compared to the non-insect passaged parent strain as measured by ST50. The LD50 remained higher than wild type, but was lower than that of EVG016. An explanation for these results is that the increased virulence of EVG017g was acquired during passage through the insect rather than during the thermal adaptation. Another possibility is that the increased infectivity (ST50) is an independent trait that arose in the lineage prior to the isolation of EVG016. Another possibility is that the enhanced infectivity is linked to the thermotolerant trait. These results suggest virulence can be recovered following loss due to the thermal adaptation protocol.

It is intriguing to speculate that the changes we measured in virulence parameters are related to the acquisition of thermotolerance. To test this, we reared the infected M. sanguinipes at 36-37° C. to mimic the insects' ability to thermoregulate to a temperature that is the new upper threshold of the evolved strains. Measurements of body temperature revealed that the insects maintained a constant body temperature that was in equilibrium with the cage temperature (36-36.5° C.). Despite their confirmed thermotolerance, the adapted variants did not show increased virulence at 36-37° C., indicating that the ability to grow in vitro at 36-37° C. does not necessarily mean that in vivo growth and pathogenesis will occur.

It is likely that more than one evolutionary pathway to thermotolerance exists and the continuous culturing system described herein could be used to probe this interesting question. Essentially, the continuous culturing system described herein selects for variants with positive growth rates over those with zero or negative growth rates. During our adaptation experiment it was noted that it takes longer for favorable variants to take over during certain cycles, appearing to indicate that the evolution is occurring in discrete steps, although this may be inaccurate. For example, we observed that for most incremental increases in temperature, the selection for faster growing variants was rapid and took roughly the same amount of time. However, at certain temperatures (32° C., 36.5° C. and 37.5° C.), it took longer for favorable variants to take over, hence these temperatures were considered as thermal barriers, perhaps requiring multiple or complex mutations to arise in the population. It is possible that a different evolutionary pathway might encounter different thermal barriers.

### Example 3

### Artificial Evolution of a Bacterium

Strains and media: The input strain MG1655 was obtained from the Escherichia coli Genetic Stock Center (CGSC, Yale, Conn.). LB and M9 minimal media were made according to standard protocol known in the art (e.g. Sambrook et al., Molecular Cloning: A Laboratory Manual, Cold Spring Harbor Laboratory Press, Third Edition (2001). Carbon sources were all used at a final concentration of 0.4% (w/v). E. coli K-12 MG1655 was inoculated into the growth chamber containing LB and the temperature was slowly increased from 44° C. to 49.7° C. over the course of 8 months of automated dilution cycles.

Experimental Evolution: Strains were evolved according to methods, devices, and compositions described herein. Over the course of the experiment, four thermotolerant strains (EVG1031, EVG1041, EVG1058 and EVG1064) were sequentially taken from the Evolugator™ at various temperatures and cryogenically stored for further study. Directed selection occurs inside a growth chamber made of flexible, translucent and gas-permeable tubing. The tubing was filled with the appropriate growth medium and autoclaved prior to inoculation of 2 mL of a growing culture of the strain to be evolved via injection through the tubing with a syringe. The tubing was subdivided using clamps that prevented the flow of medium and cultured organisms from one subdivision to the next. Actively growing culture was contained in the growth chamber. Upstream of the growth chamber was fresh medium and downstream was saturated culture. Oxygenation of the growth chamber was maintained by a bubble of filtered air in the growth chamber and agitation was achieved by rocking the chamber back and forth. A fresh bubble was delivered with each dilution cycle by movement of air in predetermined volumes through the unused portion of media upstream of the growth chamber. Dilutions were conducted automatically and controlled through specifically designed software. The clamps were periodically released, the tubing moved and the clamps reclosed. During this process, half of the growth chamber and culture were removed by peristaltic action and the remainder was mixed with fresh medium. After the clamps reclose, samples were taken directly from the tubing downstream of the growth chamber using a sterile syringe without affecting the population in the growth chamber. Turbidimeters continually measured the optical density through the tubing and were zeroed with unused growth medium prior to each experiment. The entire growth chamber was encased in an environmentally controlled box in which temperature was monitored using a PT100 probe (IEC/Din Class A), and regulated via a Proportional Integral & Derivative controller (West™ P6100).

A culture chamber was filled with LB medium and inoculated with a preculture of MG1655 grown in LB overnight. Over the course of 8 months, the temperature of incubation chamber was gradually increased from 44° C. to 49.7° C. Growth curves were closely monitored to ensure dilution during logarithmic growth. Occasionally, upon an increase in temperature, optical density was not changed, indicating that variants with adaptive mutations had not yet arisen in the population. Under these circumstances, the temperature was decreased to allow the culture to recover and adaptive strains to arise before continuing the increase in culture temperature. Samples were periodically taken during the adaptation process and cryogenically stored (−80° C.). When an increase in temperature killed the culture, the last frozen strain was re-streaked from collection onto LB plates at 37° C. and re-inoculated into the growth chamber at or below the Tmax of the frozen strain.

Genome Sequencing: Genomes of evolved strains were sequenced using the Solexa/Illumina sequencing platform. Briefly, genomic DNA preparations were made using DNEasy kit (Qiagen™). Genome libraries of each strain were generated using the Genomic DNA sample prep kit (Illumina™) as described by the manufacturer's directions. Sequencing was performed in a 36 cycle single end run (Core Facility, Oregon State University). SNPs were identified using both CLC genomics workbench v3.6.5 (CLC Bio™, MA) and Maq™ program. SNPs were independently verified by Sanger sequencing (University of Florida Core Sequencing Facility). Primers used for confirmation of SNPs by Sanger sequencing are listed in Table 5.

TABLE 5
Oligonucleotides used to amplify specific regions from the relevant
strain's chromosome for the purpose of Sanger sequencing

| Oligonucleotide | Sequence (5′→3′) | Oligonucleotide | Sequence (5′→3′) |
|---|---|---|---|
| rpod_fwd | atggagcaaaacccgcagtc | malt_fwd | atgctgattccgtcaaaact |
| rpod_rev | ttaatcgtccaggaagctac | malt_rev | ttacacgccgtaccccatca |
| ylbe_fwd | atgtttacatcagtggcgca | yhhz_fwd | atgagtaatattgtttacct |
| ylbe_rev | tcacttcccctgctccagta | yhhz_rev | tcattttgtgtggtccataa |
| kdpd_fwd | atgaataacgaacccttacg | mals_fwd | atgaaactcgccgcctgttt |
| kdpd_rev | tcacatatcctcatgaaatt | mals_rev | ttactgttgccctgcccaga |
| ybhn_fwd | atgagtaaatcacacccgcg | spot_fwd | ttgtatctgtttgaaagcct |
| ybhn_rev | tcacatcgccgcttcatttt | spot_rev | ttaatttcggtttcgggtga |
| rpsa_fwd | atgactgaatcttttgctca | wzze_fwd | atgacacaaccaatgcctgg |
| rpsa_rev | ttactcgcctttagctgctt | wzze_rev | ctatttcgagcaacggcggg |
| pncb_fwd | atgacacaattcgcttctcc | rfft_fwd | atgactgtactgattcacgt |
| pncb_rev | ttaactggcttttttaatat | rfft_rev | tcatgcgacctccctggcgg |
| faba_fwd | atggtagataaacgcgaatc | glpf_fwd | atgagttaaacatcaacctt |
| faba_rev | tcagaaggcagacgtatcct | glpf_rev | ttacagcgaagctttttgtt |
| yddb_fwd | atgaagcgagttcttattcc | treb_fwd | atgatgagcaaaataaacca |
| yddb_rev | ttaaaatttcatgctgacat | treb_rev | ttaaacaatgtccagcgtgc |
| dgsa_fwd | gtggttgctgaaaaccagcc | idi_fwd | atgcaaacggaacacgtcat |
| dgsa_rev | ttaaccctgcaacagacgaa | idi_rev | ttatttaagctgggtaaatg |
| pykf_fwd | atgaaaaagaccaaaattgt | yidE_upstream_fwd | ccaatacctaatcctatgcc |
| pykf_rev | ttacaggacgtgaacagatg | yidE_upstream_rev | tcgtaaacggtttactgcat |
| yejm_fwd | atggtaactcatcgtcagcg | ppiC_upstream_fwd | agcttgccgaaatcggcccc |
| yejm_rev | tcagttagcgataaaacgct | ppiC upstream_rev | cttacagagggtatcttaat |
| tktb_fwd | atgtcccgaaaagaccttgc | yegTfbaB_upstream_fwd | tcatgtccggggagataaag |
| tktb_rev | tcaggcacctttcactccca | yegTfbaB_upstream_rev | aaaccgcttttacttaacca |
| mred_fwd | gtggcgagctatcgtagcca | rydC_upstream_fwd | cgcatgatgccgcgtaaacg |
| mred_rev | ttattgcactgcaaactgct | rydC_upstream_rev | tgtgagatcccccctttcga |
| rpsj_fwd | atgcagaaccaaagaatccg | yajD_upstream_fwd | tggcatctgcgttggctctg |
| rpsj_rev | ttaacccaggctgatctgca | yajD_upstream_rev | aactcgcgggaacagcgacc |
| perr_fwd | atgaagctcttagcaaaagc | gltP_upstream_fwd | tatggcaaaaagtgatggat |
| perr_rev | tcaacgaattttacccagat | gltP_upstream_rev | tcgcggctgtcgctatggta |
| malq_fwd | atggaaagcaaacgtctgga | yqjF_upstream_fwd | atcctaatatgctggtccgc |
| malq_rev | ctacttcttcttcgctgcag | yqjF_upstream_rev | gtacccgcgtagccagtaat |

Upon sequencing our strain of Escherichia coli K-12 MG1655 an A to G polymorphism at position 547694 of the genome (in or upstream of the ylbE gene) was identified, which differed from the published MG1655 genome sequence (Table 1). This polymorphism results in a synonymous substitution at position 114 of the ylbE gene and was retained in all strains, including EVG1064.

The sequence of an evolved thermophile is compared to the genome of its ancestral mesophile. For example, the genome of the ancestral mesophile E. coli MG1655, and the genome of the evolved thermophile EVG1065, EVG1031, EVG1041 or EVG1058 was sequenced. Without being bound by theory, the whole genome sequencing of intermediate strains (i.e., a parental strain to EVG1064) and their comparison to MG1655 allowed the correlation of thermal adaptation in each intermediate strain with the occurrence of genetic substitutions as they first appeared in each intermediate strain. This correlation provides information on the relevance of certain genes to the evolution of thermotolerance in E. coli. Further, by observing intermediate strains the order of gene mutation could be correlated with the adaptation of E. coli as it evolved from a wild type strain to the EVG1064 strain (Table 1). A comparison of MG1655 and EVG1064 revealed 31 single nucleotide substitutions that were confirmed by Sanger sequencing.

Table 1. Identification of SNPs and their evolutionary history

TABLE 1
Identification of SNPs and their evolutionary history

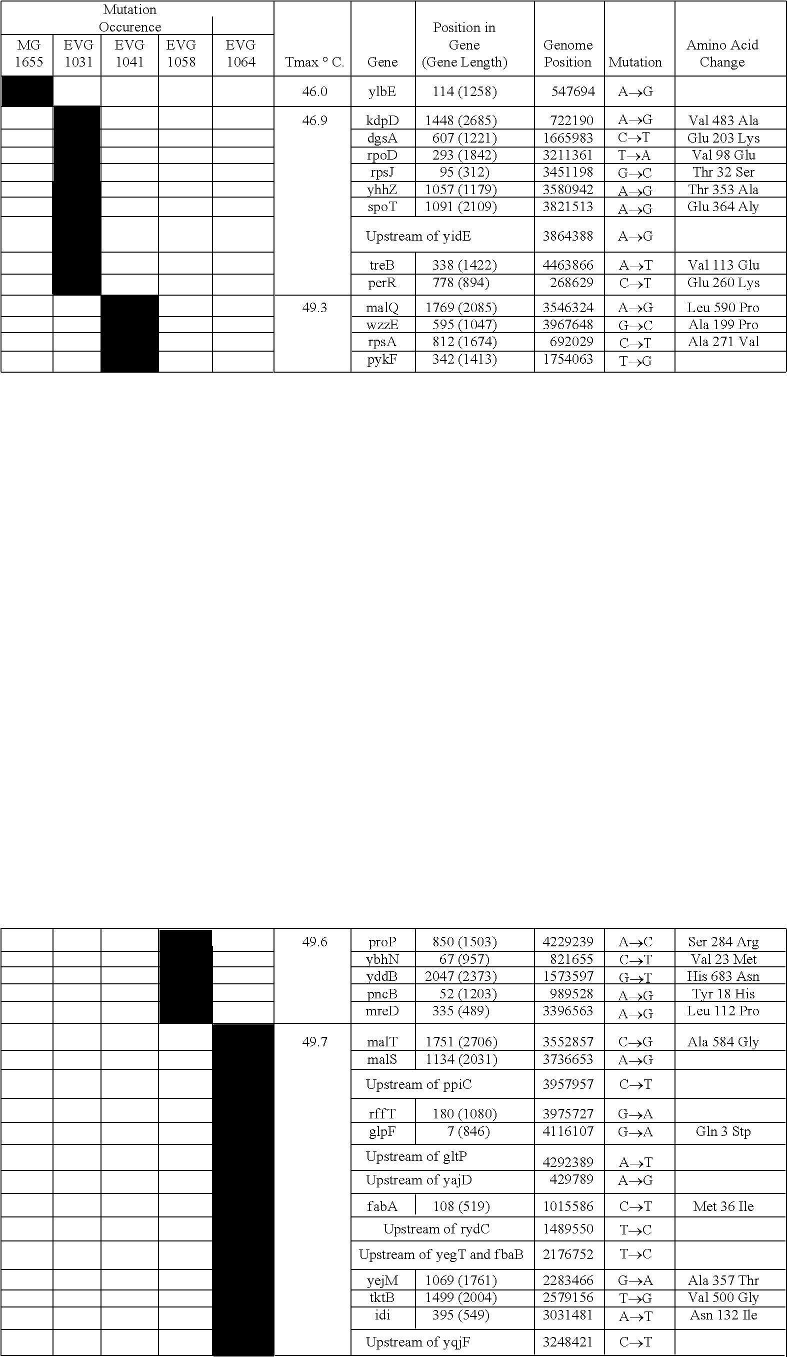

For example, as seen in Table 1, 17 substitutions were acquired and maintained through to EVG1064, indicating a high probability that these mutations are adaptive. A single additional mutation was identified during the evolutionary process that was lost prior to the isolation of EVG1064, probably due to out-competition. The 7:1 ratio of non-synonymous to synonymous mutations is indicative of a strong adaptive signal.

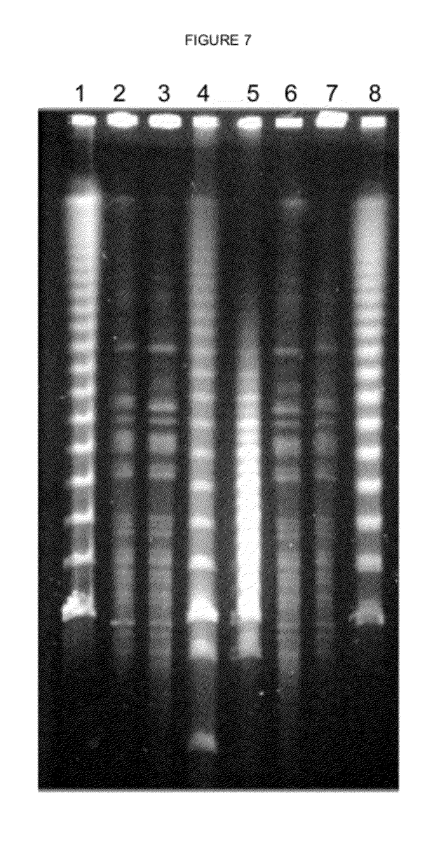

To assess the possibility of genomic rearrangements associated with thermal adaptation, MG1655 and EVG1064 were analyzed for restriction fragment length polymorphisms (RFLP) using pulsed field gel electrophoresis (PFGE). This method indicated that there were no chromosomal recombination events during strain adaption (FIG. 7).

Various mutations can occur in the process of evolving a mesophile to a thermophile. As identified here, the mutation can be a mutation in fabA gene. The fabA encodes dehydratase/isomerase responsible for the incorporation of cis-double bonds into fatty acids. FabA gene had Met36Ile mutation. Other mutations can be a mutation that would increase the degree of saturation of cis double bonds into fatty acids to maintain membrane integrity at elevated temperatures. A mutation acquired during the evolution of a mesophile to a thermophile can be a mutation on a conserved residue of a dehydratase/isomerase.

Genetic database search of the fabA family revealed that Met36 is conserved in homologs from over 300 bacterial genomes, strongly suggesting that the Met36Ile mutation affects function. The conserved residue is positioned to affect the binding pocket of fabA to a fatty acid molecule. Moreover, in the crystal structure of fabA (PDB:1MKA), Met36 is approximately 12 Å away from bound fatty acid inhibitor, in the “second shell” of atoms in contact with the substrate. This is the shell where single amino acid replacements are most likely to effect subtle changes in enzyme specificity.

Phenotypic Analysis: For liquid growth curves, overnight cultures were grown in LB at 37° C., normalized for optical density and reinoculated into medium that had been pre-equilibrated at either 37° C. or 48° C. Growth was monitored by measuring OD₆₀₀. Doubling times were determined by plotting ln OD₆₀₀ v. time and measuring the slope of the line during logarithmic phase. Doubling time=ln 2/slope. Estimated lag time was determined by time required for the culture to enter logarithmic growth. Growth curves were performed in a shaking incubator, set to 180 rpm (Multitron incubator, Infors™). Thermotolerance on solid media plates was assessed by growing streaks of the relevant strains on LB agar at 37° C. and re-streaking onto plates that had been pre-equilibrated at either 30° C., 37° C. or 48.5° C. Plates were incubated in a UVP SI-950 high-thermal accuracy incubator. Temperature variation was kept to a minimum in all incubators by pre-equilibrating to the desired temperature at least 24 hours in advance and dedicating an incubator to each experiment to limit door opening.

For thermal killing assays, overnight cultures were grown at either 37° C. (MG1655) or 47° C. (EVG1064) without agitation. 100 μL of each culture was pipetted into 6 PCR tubes. The tubes were placed in a BioRad™ gradient iCycler™ thermocycler and incubated for 30 minutes using a temperature gradient with 6 steps from 48° C. to 60° C. 5 μL of 1×, 0.1× and 0.01× dilutions were then spotted onto LB plates and incubated at 37° C. to recover. Due to the possibility that EVG1064 suffers from antagonistic pleiotropy, when grown at lower temperatures, the same experiment was repeated with the exception that EVG1064 was allowed to recover at 48.5° C. instead of 37° C. The results were the same regardless of recovery temperature and recovery at 48.5° C. is shown in FIG. 5C.

As methods, devices, and compositions described herein provide evolutionary pressure to acquire certain trait, but do not provide a particular evolutionary path, an evolutionary path taken by an evolving mesophile can bifurcate or differ from another evolutionary path taken by another evolving mesophile even if both mesophiles are evolved under the same continuous culture condition. For example, under the evolutionary pressure to acquire thermophily, some mesophile can also acquire a tropism toward a certain culture medium. A thermophile can show a tropism toward a certain culture medium. As shown here, EVO 1031 grows well in LB medium, but not in M9 minimal medium is EVG1031. Another example of nutrient tropism is EVG1041, EVG1058, or EVG1064. The EVG1031 strain has lost the ability to grow on M9 minimal medium with maltose as the sole carbon source (Table 2).

The traits identified in Table 2 play a role in long-term adaptation to LB medium, which is carbon-limited due to the lack of carbohydrates. One or more mutated genes identified here, such as pykF, dgsA, spoT and malT, can be involved in long term adaptation to glucose limitation. Mutations acquired in the EVG1031 strain are related to adaptation to a carbon source.

TABLE 2
Growth of wild type and thermotolerant mutant strains on M9 minimal medium with various carbon sources ±
aromatic amino acid and vitamin supplementation.

| | 30° C. | | 37° C. | | | | | 43° C. | | | | | 46° C. | | | | | 48.5° C. | | | | |
|---|---|---|---|---|---|---|---|---|---|---|---|---|---|---|---|---|---|---|---|---|---|---|
| | − | | − | | | + | | − | | | + | | − | | | + | | − | | | + | |
| Suppl. | D | M | D | G | M | D | G | D | G | M | D | G | D | G | M | D | G | D | G | M | D | G |
| MG1655 | + | + | + | + | + | + | + | + | + | + | + | + | − | − | − | − | − | − | − | − | − | − |
| EVG1031 | + | − | + | + | − | + | + | + | + | − | + | + | − | − | − | − | − | − | − | − | − | − |
| EVG1041 | + | − | + | + | − | + | + | + | + | − | + | + | + | + | − | + | + | − | − | − | − | − |
| EVG1058 | + | − | + | + | − | + | + | + | + | − | + | + | + | + | − | + | + | − | − | − | − | − |
| EVG1064 | − | − | − | − | − | + | − | − | − | − | + | − | − | − | − | + | − | − | − | − | − | − |

D = Glucose (dextrose),
G = glycerol,
M = maltose.

EVG 1031 showed adaptation to glucose-limiting medium. The mutations involved in this adaptation were found in genes including pykF, dgsA, spoT, malT, tktB (transketolase B) and glpF (aquaglyceroporin).

EVG1031 showed carbon-source adaptation, such as growing on LB plates. EVG1064 strain showed mutations in genes related to carbon source utilization, such as mutations in the tktB (transketolase B) or glpF (aquaglyceroporin) genes. In one case, a tktB mutation results in loss of transketolase activity. In another case, the EVG1064 strain did not grow on minimal medium with glucose as the carbon source at any temperature unless certain aromatic amino acids and vitamins for which transketolase null mutants are known to be auxotrophic are added to the medium (Table 2). In another case, neither EVG1058 nor EVG1064 grew at 48.5° C. in minimal medium, even with aromatic amino acid and vitamin supplementation. EVG1058 or EVG1064 has acquired temperature-sensitive auxotrophy. The mutation in glpF, which is required for glycerol utilization, yields a premature stop codon at position 3 that results in a non-functional protein. In one case, EVG1064 could not utilize glycerol as a carbon source (Table 2).

Fatty acid methyl ester analysis (FAME) was performed. Briefly, EVG1058 and EVG1064 were streaked onto LB agar plates and grown at 48° C. Following 24 hours of growth the plates were provided to the laboratory where transesterification and analysis by GC was performed using the Sherlock System developed by MIDI, Inc. Fatty acids that consistently comprise >1% of the total are included in Table 3. All saturated and unsaturated fatty acids were included in the calculation of saturated/unsaturated ratio. Summed features are groups of two or three fatty acids that cannot be separated by GC with the MIDI system. Summed feature 2 contains C14:0 3-OH, C16:1 iso I or both. Summed feature 3 contains C16:1ω7c and/or C15:0 iso 2-OH. Summed feature 3 in the chromatogram was assigned to the abundant fatty acid C16:1 Δ9c fatty acid C15:0 iso 2-OH co-elutes were also detected. Summed feature 2 in the chromatogram was assigned to C14:0 3-OH, which was an abundant component of E. coli lipid A. Fatty acid C16:1 iso I co-elutes were also detected. This experiment was performed in triplicate and values were reported ±1 standard deviation. Unpaired t-tests were calculated using Microsoft Excel with one-tail and unequal variance (type 3).

Growth on various carbon sources was determined by re-streaking single colonies from LB agar plates grown at 37° C. onto plates that were pre-equilibrated at various temperatures. Plates contained either LB agar or minimal M9 agar supplemented with glucose (dextrose), glycerol or maltose as carbon source. Aromatic amino acid and vitamin supplements include 500 μM L-phenylalanine, 250 μM L-tyrosine, 200 μM L-tryptophan, 6 μM p-aminobenzoate, 6 μM p-hydroxybenzoate, 50 μM 2,3-dihydroxybenzoate, 10 μM pyridoxal and 100 μM glycolaldehyde.

Fatty acid composition can be affected by the artificial evolution process described herein. A semi-quantitative comparison of fatty acids at 48° C. shows significantly higher ratios of saturated/unsaturated fatty acids in EVG1064 when compared to EVG1058. (Table 3) This difference is largely due to significantly more palmitate (C16:0) and significantly less cis-palmitoleate (C16:1 Δ9c) and cis-vaccenate (C18:1 Δ11c).

TABLE 3
FAME analysis of lipids from EVG1058 and EVG1064 grown at 48° C.
Fatty Acid (% of total fatty acid methyl esters)

| @ 37° C. | C12:0 | C14:0 | C14:0 3OH | C16:0 |
|---|---|---|---|---|
| EVG1058 fabA WT | 4.6 ± 0.3 | 9.4 ± 1.6 | 9.7 ± 0.4 | 30.1 ± 1.3 |
| EVG1064 fabA M36I | 4.4 ± 0.2 | 9.8 ± 1.4 | 9.3 ± 0.9 | 30.8 ± 2.4 |
| | C16:1 Δ9c | C18:1 Δ11c | cyC17 Δ9 | |
| EVG1058 fabA WT | 13.8 ± 2.6 | 11.5 ± 0.8 | 15.6 ± 2.6 | |
| EVG1064 fabA M36I | 11.3 ± 5.2 | 9.1 ± 1.8 | 18.4 ± 4.8 | |
| @ 48° C. | C12:0 | C14:0 | C14:0 3OH | C16:0 |
| EVG1058 fabA WT | 6.3 ± 0.6 | 11.4 ± 1.0 | 13.4 ± 2.5 | 36.3 ± 2.8** |
| EVG1064 fabA M36I | 5.0 ± 0.4 | 12.2 ± 0.0 | 11.3 ± 1.5 | 41.6 ± 1.3** |
| | C16:1 Δ9c | C18:1 Δ11c | cyC17 Δ9 | |
| EVG1058 fabA WT | 16.1 ± 0.8** | 6.7 ± 2.4** | 7.0 ± 2.3 | |
| EVG1064 fabA M36I | 13.4 ± 1.1** | 2.8 ± 0.3** | 11.1 ± 1.2 | |

| | 37° C. | 48° C. |
|---|---|---|
| | Saturated/Unsaturated | Saturated/Unsaturated |
| EVG1058 fabA WT | 2.2 ± 0.3 | 3.0 ± 0.3* |
| EVG1064 fabA M36I | 2.9 ± 0.9 | 4.5 ± 0.3* |

Some mesophiles can show antagonistic pleiotropy after evolved to a thermophile. The antagonistic pleiotropy observed from an evolved thermophile can be its lowered resistance to thermal growth inhibition. For example, while capable of growing robustly at temperatures that are restrictive for the wild type, the growth of EVG1064 can be significantly inhibited by exposing EVG1064 to about 53° C. for 30 minutes. In contrast, ancestral MG1655 can sustain 30 minutes at about 56° C. (FIG. 5D).

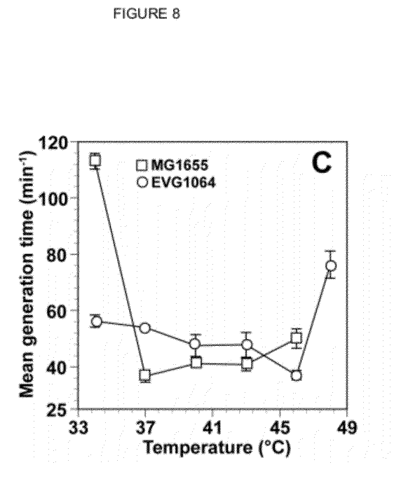

Mean generation times for MG1655 and EVG1064 were determined in batch LB culture at various temperatures to determine Tₒₚₜ (FIG. 8). The Tₒₚₜ for wild type is approximately 37° C. On the other hand, the Tₒₚₜ for EVG1064 increased to greater than 45° C., demonstrating an increase in optimal growth temperature as well as maximal growth temperature.

### Example 4

### Adaptation of a Fungal Strain for Enhanced UV Tolerance

M. anisopliae strain ATCC22099 will be obtained from American Tissue Culture Collection (ATCC). The strain will be grown on agar medium containing 2% (w/v) sucrose for 4-5 days at 35° C. Conidia will be harvested from the plate. Conidial suspensions will then be prepared in a liquid medium. The suspended culture will be introduced to a continuous culture device. The culture will be grown to O.D. 0.6-0.8. To determine an initial dose of UV, the culture will be sampled. The sample will then be filtered and adjusted to a pre-determined concentration with the use of a hemocytometer. Approximately same number of cells will be spotted on agar medium. The cells will then be grown for a few hours and exposed to various amounts of UV-B radiation. LD50 (the median lethal dose) will be calculated by counting the number of colonies. Once LD50 will be determined, an initial dose of UV-B will be set to 1/100 to 1/1000 of LDS°. The culture will be exposed to an initial dose of UV-B and will be sampled periodically to determine enhanced tolerance to UV-B light.

### Example 5

### Adaptation of an E. coli Strain for Enhanced Host Specificity

An E. coli strain will be purchased from ATCC. The strain will be grown on LB-agar medium for one day at 37° C. Colonies are harvested from the plate. Individual colonies will be separately seeded to a liquid LB-medium. The culture will be grown to a stationary phase and then introduced to a larger volume of media in a continuous culture device. The culture will be grown to O.D. 0.6-0.8. To determine an initial dose of UV, the culture will be sampled. The sample will be exposed to various amounts of UV-B radiation. After the radiation, the same volume of liquid culture will be spotted on an LB-agar plate. The plate will be incubated for a day and LD50 (the median lethal dose) will be calculated by counting the number of colonies. Once LD50 is determined, an initial dose of UV-B will be set to 1/100 to 1/1000 of LD50. The liquid culture will be exposed to an initial dose of UV-B and will be sampled periodically to determine enhanced tolerance to UV-B.

### Example 6

### Adaptation of a Bacterial Strain for Host Specificity

A strain of B. thuringiens will be will be purchased from ATCC. The strain will be first expanded in a liquid media. The expanded strain will be then grown in a media containing a mixture of growth medium and caterpillar extract in a continuous culture device. Over the course of culture, the amount of caterpillar extract will be increased while the amount of growth medium will be decreased. To increase diversity of the host specificity beyond the caterpillar stage, caterpillar extracts are admixed with biological material obtained from adult moths. The culture will be continuously exposed to increasing amount of caterpillar extracts as well as increasing amount of biological material from moths. Adaptation to changing media composition will be monitored by measuring growth characteristics such as Tₘₐₓ. The process will be repeated iteratively until complete adaptation to growth on adult moth material will be achieved.

### Example 7

### Field Application of EMO Strains

To granulate, solid medium inoculated with adapted M. anisopliae will be heated in a dry oven at 70° C. for 2 hours. After the drying, the dried medium will be pulverized to powder form followed by adding 3% surfactants, 2% adjuvants and 10-30% diluents to the above 30-50% raw-powders. The mixture will be kneaded with 35% water. The kneaded dough will be then granulated by passing through a Basket type extruder. Granules are then dried in a dry oven at 70° C. Dusts are removed by sieving the dried materials with a 16-30 mesh sieve. Granules are then packaged in a sealed pouch for manual or automatic application to a field.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-217. (canceled)

218. A method of controlling a pest comprising:
(a) applying a microorganism artificially evolved to acquire one or more traits that increase said microorganism's ability to inhibit a pest that to an area affected by pest infestation; and
(b) inhibiting said pest with said microorganism.

219. The method of claim 218, wherein said microorganism is a bacterium.

220. The method of claim 218, wherein said microorganism is a fungus or entomopathogenic fungus.

221. The method of claim 218, wherein said one or more traits comprise enhanced tolerance to ultraviolet light, enhanced tolerance to a chemical, enhanced thermotolerance, enhanced growth rate on a target carbon source, enhanced growth rate on a target nitrogen source, or modified sporulation characteristics.

222. The method of claim 218, wherein said microorganism is Metarhizium anisopliae, Metarhizium flavoridae, or Beauveria bassiana.

223. The method of claim 218, wherein said microorganism is Escherichia coli.

224. The method of claim 218, wherein the rate of growth of said microorganism at 35.5° C. exceeds that of a naturally occurring strain.

225. A method of artificially evolving a microorganism to acquire one or more traits, wherein said one or more traits comprise enhanced tolerance to ultraviolet light, enhanced tolerance to a chemical, enhanced thermotolerance, enhanced growth rate on a target carbon source, enhanced growth rate on a target nitrogen source, or modified sporulation characteristics, said method comprising:
(a) administering a microorganism into a flexible tubing wherein said tubing is subdivided by an operation of a gate into one or more discreet chambers;
(b) placing said microorganism under one or more culture conditions, wherein said one or more culture conditions comprise exposure to ultraviolet light, exposure to said chemical, exposure to a higher or lower temperature than at which said microorganism typically grows, conditions that enhance said microorganism's growth rate on said target carbon source, conditions that enhance said microorganism's growth rate on said target nitrogen source, or exposure to conditions that modify said microorganism's sporulation characteristics or spores;
(c) allowing said microorganism to grow continuously in said chamber under said one or more culture conditions until said microorganism has evolved said one or more traits that are not naturally associated with said microorganism.

226. The method of claim 225, wherein said microorganism is a bacterium.

227. The method of claim 225, wherein said microorganism is a fungus.

228. The method of claim 225, wherein said microorganism an entomopathogenic fungus.

229. The method of claim 225, wherein said microorganism is Metarhizium anisopliae, Metarhiziwnflavoridae, or Beauveria bassiana.

230. The method of claim 225, wherein said microorganism is Escherichia coli.

231. The method of claim 225, wherein at least one of said traits is enhanced tolerance to a chemical and wherein said chemical is a fungicide.

232. The method of claim 225, wherein said at least one of said one or more traits is enhanced thermotolerance and wherein said higher or lower temperature ranges from about 5° C. to about 70° C.

233. The method of claim 225, wherein said at least one of said one or more traits is enhanced thermotolerance and wherein said enhanced thermotolerance is the ability to grow at higher temperatures than said microorganisms normal range.

234. The method of claim 225, wherein at least one of said one or more traits is enhanced growth of a target carbon source and wherein said target carbon source comprises components of a host insect.

235. The method of claim 225, wherein at least one of said one or more traits is enhanced growth of a target nitrogen source and wherein said target nitrogen source comprises components of a host insect.

236. The method of claim 225, wherein at least one of said one or more traits is modified sporulation characteristics and wherein said microorganism is induced to form spores and wherein said inducing comprises drying out said chamber.

237. A method of artificially evolving a strain of Metarhizium anisopliae, Metarhiziwn flavoviridae, or Beauveria bassiana to enhanced thermotolerance by continuously culturing said strain under a condition, wherein said condition comprises incrementally increasing culture temperature, wherein said strain grows robustly at 37° C., and wherein said strain inhibits grasshoppers, locusts, cockchafers, grubs, borers or malaria-vectoring mosquitoes infestation.
